(19) United States
(12) Patent Application Publication
(10) Pub. No.: US 2026/0294622 A1
(43) Pub. Date: Oct. 1, 2026

(54) MEDICAL IMPLANT HAVING OUTER SKIRT WITH 3D STRUCTURE

(71) Applicant: BIOTRONIK AG, Buelach (CH)

(72) Inventors: Giuseppe Pisani, Buelach (CH); Matteo Astorino, Buelach (CH); Alexander Rzany, Nuernberg (DE); Sabine Weingaertner, Erlangen (DE); Bernhard Hensel, Erlangen (DE); Kilian Badrutt, Kloten (CH); Davide Mogentale, Rafz ZH (CH); Nadine Troes, Buelach (CH); Patrik Markovic, Birmensdorf (CH)

(21) Appl. No.: 19/704,640

(22) Filed: Jun. 11, 2026

Related U.S. Application Data
(63) Continuation of application No. 18/008,723, filed on Jun. 9, 2021.

(30) Foreign Application Priority Data
Jun. 9, 2020 (EP) 20178948.4
Oct. 30, 2020 (EP) 20205070.4

(51) Int. Cl. A61F 2/24

(52) U.S. Cl. CPC A61F 2/2418; A61F 2/2415

(57) ABSTRACT

A medical implant for transcatheter delivery to an implantation site. The medical implant includes an implant component defining a flow passage. The flow passage has an inflow end and an outflow end. The implant component includes a stent-structure that has compressed state and is expandable into functional state. A plurality of cells is between first and second ends of the stent structure. An outer skirt at least partially covers an outer side of the stent-structure. A suture affixes the outer skirt to the stent structure. At least in the functional state, the outer skirt comprises a plurality of protrusions forming a seamless 3D structure extending radially outward from a flat portion of the outer skirt. The flat portion abuts the stent-structure.

### PRIORITY CLAIM AND REFERENCE TO RELATED APPLICATION

The application is a continuation of and claims priority under 35 U.S.C. § 120 and all applicable statutes and treaties from prior pending U.S. application Ser. No. 18/008,723, filed Dec. 7, 2022, which application is a 35 U.S.C. 371 US National Phase and claims priority under 35 U.S.C. § 119, 35 U.S.C. 365(b) and all applicable statutes and treaties from prior PCT Application PCT/EP2021/065510, which was filed Jun. 9, 2021, which application claimed priority from European Application Number 20178948.4, which was filed Jun. 9, 2020 and from European Application Number 20205070.4, which was filed Apr. 27, 2020. All applications mentioned in this paragraph are incorporated by reference.

### FIELD

A field of the invention concerns medical implants, such as, but not limited to a vascular implant, preferably a transcatheter deliverable endoprosthesis e.g. a prosthetic venous valve or a prosthetic heart valve for valve replacement. A particular application of the invention is to a transcatheter deliverable endoprosthesis e.g. a prosthetic heart valve used in the treatment of a stenotic cardiac valve and/or a cardiac valve insufficiency, for example in a transcatheter aortic valve replacement (TAVR) or in a transcatheter aortic valve implantation (TAVI). A prosthetic heart valve may also be referred to a stented valve or valved-stent.

### BACKGROUND

Conventional approaches for cardiac valve replacement require the cutting of a relatively large opening in the patient's sternum (“sternotomy”) or thoracic cavity (“thoracotomy”) in order to allow the surgeon to access the patient's heart. Additionally, these approaches require arrest of the patient's heart and a cardiopulmonary bypass (i.e., use of a heart-lung bypass machine to oxygenate and circulate the patient's blood). In recent years, efforts have been made to establish a less invasive cardiac valve replacement procedure, by delivering and implanting a cardiac replacement valve via a catheter inserted through a smaller skin incision via either a transvascular approach; i.e., delivering the new valve through the femoral artery, or by transapical route, where the replacement valve is delivered between ribs and directly through the wall of the heart to the implantation site.

Stent valves and delivery systems for placing a replacement valve via a catheter are known in the art, and are disclosed for example in WO 2007/071436 and WO 2009/053497. Some known stents are made from a shape memory material, such as Nitinol, and are self-expanding. The valves may be from animals, for example porcine aortic valves. Alternatively, the valves may at least partly be made of synthetic material, such as Dacron.

Minimally-invasive treatments for implanting implant s are nowadays quite common and allow the implantation under local anesthesia. One approach provides for the use of a catheter system to introduce the medical implant including a supporting stent-structure (stent) and leaflets.

Such implant may be crimped and then in a compressed state guided via a catheter system to the implantation site, e.g. within the heart through an inguinal artery or vein. After reaching the implantation site, the medical implant may then be unfolded.

For example, WO 2007/071436 discloses a prosthetic heart valve including a valve element and a stent element. The stent element includes three different sections, wherein one section houses the valve element. The valve element includes three leaflets, which may be made of biological or artificial material. The three different sections may be provided with different diameters. Other prosthetic heart valves are described in the documents WO 2011/147849 A1 and US 2017/0065408 A1.

One major drawback of some known replacement valve stents is that even in a collapsed (crimped) state their diameter is often too big for transvascular delivery of the stent. Transfemoral delivery of the stent, where the stent has to be advanced over the aortic arch, requires even smaller diameters, e.g., of 18 French or less than 18 French (18 F equals to 6 mm). Such small diameters may also be useful in transapical delivery if a smaller skin incision and/or smaller cut in the heart wall may be used.

Crimping some known stent valves to a diameter of, e.g., 18 French or less would produce high strains on the replacement valve, which may lead to damages.

Thus, there is a need for prosthetic heart valves, which avoid the disadvantages of the known ones and which in particular may be crimped to small diameters without the risk of damaging the replacement valves and which may be reliably placed and tightly anchored over an aortic annulus.

Another problem with medical implants, such as transcatheter deliverable endoprosthesis, preferably prosthetic heart valves, is the sealing towards the anatomical structure at the implantation site. For example, for current transcatheter prosthetic heart valves paravalvular leakage (PVL) is a major drawback. Paravalvular or para-prosthetic leakage refers to blood flowing through channels between the medical implant component of the implanted transcatheter heart valve and the patient's anatomy at the implantation site, e.g. the native aortic annulus, as a result of inappropriate sealing. With regard to the aortic valve replacement the chronic backstream of blood results in a volume-overload of the left heart chamber. Resultant adverse events include shortness of breath, atrial fibrillation and other symptoms commonly associated with heart failure, including increased mortality. Consequently, minimization or the avoidance of PVL is a major focus in TAVI development. Advanced sealing strategies are a viable solution to reduce or even prevent TAVI related PVL.

The majority of known sealing concepts simply consist of flat pericardium or fabric strap firmly anchored in the inflow section of prosthesis (i.e. the prosthesis portion usually being in contact with the heart annulus/native leaflets). Some more complex concepts are leveraging on slacks or pockets. The known solutions are partially or not always capable to reach/fill notches or recesses or gaps and channels between the native aortic annulus and the medical implant. Other existing solutions, in which the material is willingly increased to fill those gaps, must sacrifice the overall crimping profile of the prosthesis.

Thus, there is a need for medical implants, such as transcatheter deliverable endoprosthesis, preferably prosthetic heart valves, which avoid these drawbacks.

### SUMMARY

A medical implant for transcatheter delivery to an implantation site includes an implant component defining a flow passage. The flow passage has an inflow end and an outflow end. The implant component includes a stent-structure that has compressed state and is expandable into functional state. A plurality of cells is between first and second ends of the stent structure. An outer skirt at least partially covers an outer side of the stent-structure. A suture affixes the outer skirt to the stent structure. At least in the functional state, the outer skirt comprises a plurality of protrusions forming a seamless 3D structure extending radially outward from a flat portion of the outer skirt. The flat portion abuts the stent-structure..

### BRIEF DESCRIPTION OF THE DRAWINGS

Further advantages and characteristics of the present invention are described in the following description of examples and figures.

FIGS. 1A and 1B show embodiments of a leaflet,

FIGS. 2A and 2B show other embodiments of a leaflet,

FIG. 3 shows a specific embodiment of a leaflet,

FIGS. 4A and 4B show embodiments of leaflets with laser markings or laser holes,

FIGS. 5A and 5B show a schematic folding and suturing technique,

FIGS. 6A and 6B show an embodiment of an inner skirt,

FIG. 7 to 9 show embodiments of an inner skirt,

FIGS. 10A and 10B show another embodiment of an inner skirt,

FIGS. 11A and 11B show an embodiment of an outer skirt,

FIG. 12 show another embodiment of an outer skirt,

FIGS. 13A and 13B show schematic drawings of a laser-aided sewing process,

FIG. 14 show another schematic drawing of a laser-aided sewing process,

FIGS. 15A and 15B show a suture for fixing a valve assembly to a stent-structure in a side view,

FIGS. 15C and 15D show the suture of FIGS. 15A and 15B with a protecting stitch structure in a top view,

FIG. 16A to 16H show steps of a manufacturing method in a side view, wherein a valve assembly is affixed to the stent-structure,

FIGS. 17A to 17H show embodiments of heart valve prostheses,

FIGS. 18A and 18B show a detail of a medical implant having an inner skirt and an outer skirt with a loop structure,

FIGS. 18C and 18D show another embodiment of a heart valve prosthesis in a side view,

FIGS. 18E and 18F show another embodiment of a heart valve prosthesis in a side view,

FIGS. 18G and 18H show another embodiment of a heart valve prosthesis in a side view,

FIG. 19 show another embodiment of an outer skirt,

FIGS. 20A and 20B show a further embodiment of a medical implant having a 3D-shaped outer skirt,

FIG. 20C shows a photo of a TAVI/TAVR heart valve prosthesis in a functional state,

FIG. 20D shows a schematic drawing of the photo of FIG. 20C,

FIGS. 21A and 21B show an apparatus for the manufacture of a 3D-shaped outer skirt,

FIGS. 22A and 22B show a detail of a medical implant having an inner skirt and an outer,

FIGS. 22C and 22D show the overlap of an inner skirt with an outer skirt,

FIGS. 23A and 22B show an embodiment of the contours of a stent-structure,

FIGS. 24 and 25 show embodiments of stent-structures,

FIG. 26 shows a section of a first embodiment of a heart valve prosthesis in a functional state and in a side view,

FIG. 27 shows the embodiment of FIG. 26 in an intermediate step of manufacturing method in a side view,

FIG. 28 depicts an outer skirt for the embodiment of FIG. 27 in a top view,

FIG. 29 depicts an inflow section of the embodiment of FIG. 26 in a side view with an attached outer skirt,

FIG. 30 shows a first sewing pattern of the embodiment of FIG. 26 when the tubular stent-structure is unrolled,

FIG. 31 depicts a second sewing pattern of the embodiment of FIG. 26 when the tubular stent-structure is unrolled,

FIG. 32 shows a detailed view of the sewing pattern of FIG. 31,

FIG. 33 depicts a section of a second embodiment of a heart valve prosthesis in a functional state and in a side view,

FIG. 34 shows an outer skirt for the embodiment of FIG. 33 in a top view,

FIG. 35A depicts a first sewing pattern of the embodiment of FIG. 33 when the tubular stent-structure is unrolled,

FIG. 35B is an exploded view of a sewing pattern of the embodiment of FIG. 35A showing one suture when the tubular stent-structure is unrolled,

FIG. 35C is an exploded view of a sewing pattern of the embodiment of FIG. 35A showing another suture when the tubular stent-structure is unrolled,

FIG. 35D is an exploded view of a sewing pattern of the embodiment of FIG. 35A showing a further suture when the tubular stent-structure is unrolled,

FIG. 36 shows the outer skirt sutured at the stent-structure according to the sewing pattern of FIG. 35A when the tubular stent-structure is unrolled,

FIG. 37 depicts a second sewing pattern of the embodiment of FIG. 33 when the tubular stent-structure is unrolled,

FIG. 38 shows an outer skirt for a third embodiment of a heart valve prosthesis in a top view,

FIG. 39 shows a cross section of an outer skirt,

FIG. 40 depicts a pattern of laser markings at the surface of an outer skirt in a top view,

FIG. 41 shows a pattern of laser markings at the surface of an outer skirt to be sutured together in a top view,

FIG. 42 show another pattern of laser markings at the surface of an outer skirt to be sutured together in a top view,

FIG. 43 to 45 steps of a manufacturing method for an attachment of an outer skirt to a vertex of the stent-structure in form of a nutcracker eye in a side view, and

FIG. 46A to 46D show steps of another manufacturing method of an outer skirt to a vertex of the stent-structure in a perspective side view,

FIG. 47 shows an embodiment of a medical implant with a valve assembly having a 3D-shaped outer skirt,

FIG. 48 shows another embodiment of a medical implant with a valve assembly having a 3D-shaped outer skirt,

FIG. 49 shows a further embodiment of a medical implant with a valve assembly having a 3D-shaped outer skirt,

FIG. 50 shows side view of another embodiment of a TAVI/TAVR heart valve prosthesis in a functional state,

### DETAILED DESCRIPTION OF THE PREFERRED EMBODIMENTS

The present invention is directed to a medical implant, such as, but not limited to a vascular implant, preferably a transcatheter deliverable endoprosthesis e.g. a prosthetic venous valve or a prosthetic heart valve for valve replacement including tissue components of one or more leaflets and one or more skirt elements that allow for a reliable valve functioning and suitable sealing against paravalvular leakage, especially of the aortic valve. The present invention also relates to a medical implant, such as, but not limited to a transcatheter deliverable endoprosthesis e.g. a prosthetic heart valve used in the treatment of a stenotic cardiac valve and/or a cardiac valve insufficiency, for example in a transcatheter aortic valve replacement (TAVR) or in a transcatheter aortic valve implantation (TAVI). A prosthetic heart valve may also be referred to a stented valve or valved-stent.

A medical implant, such as, but not limited to a vascular implant, preferably a transcatheter deliverable endoprosthesis e.g. a prosthetic venous valve or a prosthetic heart valve is described. The prosthetic heart valve may be an aortic, mitral, tricuspid or pulmonal prosthetic heart valve.

The medical implant includes a stent-structure (also hereinafter referred to as stent structure or stent component) and one or more skirts. The stent-structure has a longitudinal axis and a circumference surrounding an inner volume (lumen) and the longitudinal axis.

One skirt at least partially covers an outer side of the stent-structure and optionally extends at least partly outside the stent-structure, preferably extends radially outward the circumference of the stent-structure. One skirt may be an inner skirt (also denoted as inner skirt element) and/or one skirt may be an outer skirt (also denoted as outer skirt element). Alternatively, the one skirt at least partially covering the outer side of the stent-structure also covers at least partially the inner side of the stent-structure. The inner skirt has a first inner skirt edge and a second inner skirt edge (being opposite to the first skirt edge). The outer skirt has a first outer skirt edge and a second outer skirt edge (being opposite to the first skirt edge).

The stent-structure is an open stent-structure preferably having cylindrical or tubular shape. The circumference in this case is regarded as the perimeter. The medical implant may include a compressible and expandable stent-structure (so that the medical implant can be delivered to an implantation site using a catheter). Thus, the stent-structure may have a compressed (delivery) state and may be expandable into a functional state.

At least a part of the stent-structure, the inner skirt and/or outer skirt and optionally a valve assembly form an implant component. The medical implant component defines a flow passage for guided and/or controlled fluid flow (e.g. blood flow). The flow passage has an inflow end and an outflow end. The bodily fluid, for example blood, flows through the flow passage in one flow direction, from the (proximal) inflow end to the (opposite) outflow end of the flow passage. The inner skirt may be surrounded by the stent-structure. The stent-structure may be surrounded by the outer skirt.

The stent-structure has a (proximal) inflow section and an outflow section and at least one intermediate section (also denoted as transition zone) arranged between said inflow and outflow section. The stent-structure has a first stent-structure end and a second stent-structure end. The first stent-structure end and/or the (proximal) inflow section may be situated at the inflow end of the flow passage or the first stent-structure end may be situated spaced apart from the inflow end of the flow passage (when looking along the longitudinal axis). The second stent-structure end and/or the outflow section may be situated at the outflow end of the flow passage or the second stent-structure end may be situated spaced apart from the outflow end of the flow passage (when looking along the longitudinal axis).

The second stent-structure end is opposite the first stent-structure end (when looking along the longitudinal axis). Thus, when implant ed as intended the first stent-structure end is passed first by the bodily fluid.

The medical implant or implant component may include the valve assembly. The valve assembly may include one or more valve leaflets (shortly named hereinafter leaflets), preferably (at least) two or three leaflets. Preferably, the flow passage of the medical implant component may include the at least one or the at least two leaflets within the flow passage. The leaflets have a flow control function, which permits the fluid flow in a first direction (from the inflow end to the outflow end) but prevents the fluid flow in a second direction being reverse to the first direction. The valve assembly may be surrounded by the stent-structure.

The stent-structure includes a plurality of cells, preferably formed by a plurality of struts.

At least a part of the cells and optionally the outer skirt form the outer contours of the medical implant.

The present invention provides for a prosthetic heart valve including a stent-structure that is characterised by a conical-convex inflow section, a cylindrical straight outflow section and a transition zone connecting the inflow and outflow section and having suitably great cells for free access to the coronary arteries and further including a valve assembly of suitable leaflet and skirt configurations that are specifically adapted to the said stent structure.

Respective aspects and embodiments of the invention are further defined below, can be derived from the appended figures and are specifically outlined in the figure descriptions.

One major aspect of the invention provides a prosthetic heart valve for heart valve replacement, including a valve assembly (and/or a tissue valve) with one or more valve leaflets, preferably two or more leaflets, more preferably exactly three valve leaflets. The term “valve assembly” is used herein to refer to the leaflets collectively, whether or not the leaflets are secured together to define a unitary valve structure independent of other components.

In accordance with the invention, the leaflets are made of pericardium tissue, preferably from porcine pericardium tissue or bovine pericardium. Porcine pericardium may be desirably thin and sufficiently durable. Bovine pericardium may be thicker and even more durable when this is desired. In one embodiment each valve leaflet includes at least two tabs. In embodiments of the invention concerned with laser-assisted sewing processes (L-ASP) exceptionally only two tabs are needed on the valve structures.

The prosthetic heart valve further includes a stent structure configured to be radially compressible into a compressed state and expandable into a functional state. The stent component includes a proximal conical and convex-shaped inflow end and a cylindrical straight outflow end, and at least one intermediate section, being it a transition zone, arranged between said inflow and outflow section. The intermediate section (transition zone) has at least two commissural posts optionally and/or generally aligned parallel to an axis spanning from the inflow end to the outflow end. The tabs of the leaflets are directly attached to the commissural posts, preferably to an attachment structure provided on said commissural posts such as one or more through holes.

The valve leaflets are configured and dimensioned such as to form a functioning replacement valve and are suitable for coaptation in the stent structure of the invention. In some embodiments, the leaflets have a straight or slightly curved upper free edge, two lateral edges and a substantially arcuate lower edge. At least one tab is arranged on each lateral edge, preferably in the area of the upper free edge of the leaflet.

In the prosthetic heart valve of the invention, the at least one, preferably at least two, more preferably at least three leaflets are positioned such that their upper free edges may be pressed together to prevent blood flow in one direction, e.g. towards the heart during diastole in the case of an aortic valve replacement, and move apart to allow blood flow in the other direction, e.g. away of the heart during systole.

In one embodiment exactly three valve leaflets are provided. This allows mimicking the natural tricuspid valve architecture, e.g., of the aortic valve. Alternatively, the prosthetic heart valve may also include more leaflets, such as four, five or more.

While it is known to use a large selection of different artificial materials for replacement valves, it is preferred that the at least two leaflets of the prosthetic heart valve according to the present invention are made of pericardium tissue. In one embodiment, the at least two leaflets are made from porcine pericardium tissue. Pericardium tissue is sufficiently thin and yet durable enough to be used as leaflet material. The porcine heart shows a lot of similarities to the human heart. Therefore, it is advantageous to use porcine pericardium tissue. Further, porcine pericardium tissue is readily available.

In one embodiment, bovine pericardium may also be used for the leaflets where even greater durability is desired, optionally at the expense of thicker tissue.

In one embodiment, the stent structure is of the self-expanding type. Such stents are known in the art and often include or are made of a shape-memory material, such as Nitinol. Alternatively, the stent component may be made of or include a plastically deformable material and may be expanded to the functional state by an external device, such as a balloon catheter.

In the compressed, e.g., the crimped state, the stent component may be inserted in the area of a heart valve of a patient, such as the aortic valve. Further, the diameter of the stent component in the compressed state is such that it may be advanced into a patient's heart through an artery, such as the femoral artery. The diameter and/or the flexibility of the stent component in the compressed state are therefore preferably such that the prosthetic heart valve may be advanced through the aortic arch.

In the functional state, the stent structure is in an at least partly expanded, or non-compressed configuration. Optionally, the stent structure defines an interior conduit space. The conduit space may be generally cylindrical and/or tubular. The valve leaflets are arranged to span the interior space within the stent structure. Once the prosthetic heart valve is positioned at a target position close to the natural valve of a patient, the stent structure is expanded to its functional state.

The natural valve leaflets of the patient may be pushed aside by the expanding stent structure. Once fully expanded, the valve assembly arranged within the stent structure will take over the function of the natural valve.

In one embodiment, the valve assembly is arranged within the said intermediate section (transition zone) of the stent structure. In one embodiment, the valve assembly is arranged within the said intermediate section (transition zone) of the stent structure in supra annular position. Optionally, the stent structure is configured such that said intermediate section (transition zone) includes a conical and/or cylindrical conduit space, optionally with a constant diameter.

In the functional state, said inflow and outflow section of the stent structure define inflow and outflow openings through or around which blood may flow in use. “Inflow section” as understood herein is the section of the stent structure where blood enters into said conduit space and/or the section of the stent structure that, in use, is upstream of the valve leaflets; for example, in the case of a semilunar and/or aortic valve, the section of the stent structure which is oriented towards the ventricle.

Accordingly, an “outflow section” as understood herein is the section of the stent structure where blood leaves said conduit space and/or the section of the stent structure that, in use, is downstream of the valve leaflets; for example, the section which is located in the artery for semilunar valves.

Said inflow and said outflow section may thereby have the same length or have different lengths. Further, said inflow and/or said outflow section may define a generally tubular conduit interior conduit space. The conduit space may be generally cylindrical. More preferably, said inflow and/or said outflow section include a generally conical conduit, i.e. a conduit with an increasing or a decreasing diameter. Alternatively, the inflow and the outflow section may include an interior conduit space of any appropriate geometric shape.

Optionally, said inflow and said outflow section may have the same maximal diameter or varying maximal diameters. A “maximal diameter” as understood herein is the largest diameter within such a section. Optionally, said inflow section has a greater maximal diameter than said outflow section. Further, said intermediate section (transition zone) has a diameter which is smaller than the maximal diameter of either of said inflow or said outflow section. In another embodiment, said intermediate section (transition zone) may have a diameter that equals to the diameter of the outflow section. Alternatively, further sections may be arranged between said inflow and/or said outflow section and said intermediate section.

Further, the stent structure includes at least one attachment element for mating engagement with a delivery device (for example, a stent holder of the delivery device). The at least one attachment element may be configured for restraining axial displacement of the stent component until the stent component is fully released. In some embodiments, the at least one attachment is provided at the outflow section. The stent structure may include any suitable number of attachment elements, for example, two, three, or more, preferably three. More preferably, the three attachment elements cant inward towards the central axis of the stent structure. The attachment elements may be spaced substantially uniformly in the circumferential direction.

In accordance with the invention, a delivery catheter may include a stent-holder provided within a stent accommodation region. The stent-holder may include a respective projection receivable within each eyelet. The projection may be dimensioned such that, when the stent structure is in its collapsed state, the projection is trapped within the eyelet and unable to pass between the adjacent struts, and/or one or more recesses or interstices for accommodating the attachment element substantially there within, at least in the collapsed state of the stent component.

The above forms can provide for a compact, yet reliable and self-opening and/or self-releasing attachment between a stent-valve and a delivery system. The provision of the attachment elements also does not impede compressing of the stent component to a desirably small size.

In some embodiments, the intermediate section of the stent structure includes at least two commissural posts, preferably three commissural posts, generally aligned parallel to an axis spanning from the inflow end to the outflow end. The tabs of the leaflets are directly attached to said commissural posts, preferably to an attachment structure provided on said commissural posts such as one or more through holes, preferably three through holes.

The direct attachment of said leaflets to said commissural posts provides a high strain resistance of the leaflets. Optionally, in comparison to valve replacement stents as known in the art, the direct attachment of the leaflets to the commissural posts may optionally reduce the thickness of the crimped stent element, if excess layers of tissue between the leaflets and the commissural posts capable of withstanding the strain resistance may be avoided.

The prosthetic heart valve of the invention additionally may include an inner skirt element, preferably made of pericardium tissue, more preferably porcine pericardium, and attached to the leaflets. The inner skirt element may serve to channel blood within the conduit space of the stent component, and obstruct leakage of blood through interstices of the stent component (e.g. through cells of a lattice structure).

In some embodiments, the inner skirt element may have commissural portions spaced apart by scalloped clearances (e.g. scalloped cut-outs). Each clearance is spanned by a respective valve leaflet. The lateral edges and/or lower edges of the leaflets may be attached to the inner skirt, for example, by sutures.

In some embodiments, the inner skirt element may extend towards said second end, said skirt preferably being sutured to said stent device. Said skirt preferably covers at least partly an interior surface of the stent component. This reduces the occurrence of turbulent flow of the blood, which may be triggered by the material of the stent component. Said skirt preferably is further sutured to said at least two valve leaflets.

Additionally, at least one section of said stent structure is at least partially covered on the outside by an outer skirt element.

According to still another aspect of the invention there is provided a prosthetic heart valve for heart valve replacement including a stent structure having at least one section defining an at least partially conical body. The device further has a plurality of valve leaflets. An inner skirt is disposed within the stent component overlapping said at least partially conical body to define a conduit therewithin. An outer skirt is disposed outside the stent component overlapping only a portion of said at least partially conical body.

The inner skirt and/or the outer skirt are preferably made of pericardium tissue, most preferably porcine pericardium tissue.

In all embodiments, the tabs of the at least one, preferably at least two, more preferably at least three leaflets are sutured to the commissural posts through the holes of the commissural posts.

Another aspect of the invention provides a prosthetic heart valve including a stent structure that is radially compressible to a compressed state for delivery and radially expandable to a functional state, a plurality of valve leaflets mounted within the stent component, an inner skirt element attached to the valve leaflets, the inner skirt element extending at least partly within the stent component, and an outer skirt element extending at least partly outside the stent component.

In some embodiments, the outer skirt element may extend further towards an inflow extremity of the stent structure than does the inner skirt element. Additionally or alternatively, the inner and outer skirts may partly overlap, at least with respect to the surface of at least one of the skirts. Additionally or alternatively, the inner and outer skirts may not have any coterminous extremity. Additionally or alternatively, the inner skirt may extend further towards an outflow extremity of the stent component than does the outer skirt.

At least a portion of the stent structure over which at least one of the skirts extends, may optionally include a lattice structure having at least one row of a plurality of cells.

A function of the inner skirt element may be to define a conduit within the stent to channel blood towards the valve leaflets, and obstruct leakage of blood through interstices of the stent component (e.g., lattice interstices). A function of the outer skirt element may be to provide a seal surface outside the stent component for sealing with surrounding tissue, to obstruct leakage at the interface with surrounding tissue. Providing both skirts may be beneficial in terms of obstructing leakage overall. By providing both skirts, with only partial overlap in an axial direction, the benefits of both skirts can be obtained, but with a reduced thickness profile in the regions where only one skirt extends. Overlapping the skirts can provide better sealing between the skirts than were the skirts to be arranged edge to edge on the interior and exterior respectively of the stent component (for example, especially bearing in mind that the stent-valve is to be deformed substantially by compression for delivery and re-expansion at implantation).

The degree of skirt overlap in the axial direction may be, for example, by at least 1 mm, or at least 2 mm, or at least 3 mm, or at least 4 mm, or at least 5 mm, or at least 6 mm, or at least 7 mm, or at least 8 mm. Additionally or alternatively, the degree of skirt overlap in the axial direction may, for example, be less than 10 mm, or less than 9 mm, or less than 8 mm, or less than 7 mm, or less than 6 mm, or less than 5 mm, or less than 4 mm. For example, the degree of skirt overlap in the axial direction may be about 4-6 mm.

At least one of the skirts (optionally each skirt) may extend a non-overlapped axial distance of at least 1 mm away from the region of overlap. The non-overlapped distance for the or each skirt may, for example, be at least 2 mm, or at least 3 mm, or at least 4 mm or at least 5 mm or at least 6 mm, or at least 7 mm or at least 8 mm or at least 9 mm, or at least 10 mm.

In some embodiments, the inflow edge or mouth of the stent component may have a zig-zag shape defined by a lattice structure of at least one row of cells. The zig-zag shape may define an alternating sequence of free apexes (e.g., at or defining an inflow extremity), and connected apexes (e.g. connected to lattice structure extending away from the inflow end towards the outflow end). In some embodiments, the inner skirt element may extend only to the connected apexes. The outer skirt may overlap the inner skirt and extend further than the inner skirt, to a level corresponding to at least some of the free apexes.

In some embodiments, the inner skirt element may extend towards the inflow extremity of the stent structure. The outer skirt may overlap only partly the inner skirt while remaining spaced from an uppermost edge of the inner skirt. The outer skirt may extend towards (or optionally to) the inflow extremity of the stent structure. The outer skirt may optionally not overlap (e.g., directly or indirectly through the stent component) any portion of the leaflets.

The inner skirt and/or outer skirt may be of any suitable material, such as pericardial tissue (e.g. porcine pericardium for thinness), PET, Dacron, etc. The inner and outer skirts may optionally be made of the same material as each other.

Another object of the present invention is to provide a delivery system for delivering the prosthetic heart valve of the present invention. The delivery system includes a flexible tubular catheter including a proximal end (or portion) and a distal end (or portion) with a connection structure (e.g. a stent holder). The delivery device further includes a prosthetic heart valve as described hereinabove. The delivery device is connected with said a connection structure such that the portion of the device adapted to be placed in or towards the ventricle is oriented towards the distal end of said catheter and the portion of said device adapted to be placed in the aorta is oriented toward said proximal end. In connection with the delivery device, the term “distal” means oriented away and the term “proximal” means oriented towards an operator of the delivery device.

In connection with the prosthetic heart valve, however, the term “distal” means oriented away from the aortic ventricle and thus oriented away from the inflow sections and the term “proximal” means oriented towards the aortic ventricle and thus oriented towards the inflow section of the stent structure.

The proximal end of the tubular catheter preferably includes a handle member for an operator. The distal end of the tubular catheter includes a connection structure (e.g. stent holder) for releasably connecting a prosthetic heart valve according to the present invention. The a connection structure may be of any suitable type. Preferably, the a connection structure are configured as pins or other projections that mate with corresponding attachment elements (e.g. hooks and/or eyelets) on the prosthetic heart valve. Upon expansion of the stent component of the replacement device, the attachment elements are released from the pins, thus uncoupling the device from the tubular catheter.

The orientation of the prosthetic heart valve on the tubular catheter allows the insertion of the device along an artery of a patient, preferably along the femoral or the subclavian artery. An arterial insertion is beneficial for some patients, as the procedure is less traumatizing than a surgical procedure. If desired, the tubular catheter may also be configured for transapical insertion.

According to still another aspect of the invention there is provided a method of replacement of a heart valve. Thereby, a delivery device as disclosed above is inserted in a compressed state to the site of a heart valve to be replaced. The sent structure is then expanded. The delivery device is optionally inserted by a flexible tubular catheter along an artery, preferably a femoral artery or a subclavian artery. Alternatively, the delivery device is inserted transapically into a ventricle of the heart.

It is another objective of the present invention to provide a method of producing a prosthetic heart valve having a reduced size when radially compressed which is quick and easy to perform.

In some embodiments, in a first step of the method of production of a prosthetic heart valve according to the present invention, one or more tubular skirt elements, preferably made of pericardium tissue, is provided. The term “tubular” has to be understood as to also encompass skirts, which are generally shaped like a cylinder or a conical frustum. It also includes skirts having elliptical cross sections, varying radii along an axis and the like. The tubular skirt preferably is made of porcine pericardium tissue.

In a next step, at least two leaflets, preferably also made of pericardium tissue are arranged adjacent to each other around the tubular skirt elements. The size of the leaflets is thereby selected such that once the leaflets are each arranged adjacent to each other, they span around the entire circumference of the tubular skirt elements. The lateral edges of said leaflets are thereby in contact at least in the area of their upper free edge.

The leaflets may be cut out of pericardium tissue. The leaflets include a free edge, which is optionally curved. The curvature may be a convex curvature. In another embodiment, the curvature may be a concave curvature. The size of the leaflets as well as the curvature of the free edge are thereby chosen in such a way as to allow the free edges to sealingly contact each other (e.g. coapt) when the stent component is in the functional state. The leaflets further include two lateral edges tapering towards a lower edge of the leaflet. The lower edge is shorter than the free edge. Preferably, said lower edge is also curved, more preferably with a convex curvature. The term “convex” is understood to define the curvature of an edge of the leaflet in relation to the surface of the leaflet. Therefore, a convexly curved edge bulges out of the leaflet.

The at least one, preferably at least two, more preferably at least three leaflets preferably additionally include at least two tabs, preferably one tab is thereby arranged on each lateral edge of each leaflet, most preferably in the area of said free edge. Alternatively, the at least two leaflets may include more tabs, e.g. two tabs on each lateral edge of each leaflet.

The tabs are then preferably directly attached to the stent structure, preferably to an attachment structure provided on the stent commissural posts, followed by suturing said tabs to said commissural posts. Superfluous material of said tabs may then be removed.

As indicated above, the leaflets and skirt elements of an artificial valve of a prosthetic heart valve need to be specifically adapted to the stent structure they will be adhered to. This is essential in order to allow for a safe and reliable functionality of the artificial heart valve. For instance, based on the general configuration of a leaflet and/or skirt design, further minor dimensional variations may be indicated in order to find the best fit to the stent structure, to optimize the opening/closure behaviour of the artificial valve and thus to optimize the relative haemodynamic performance of the said valve, and lastly to optimize the stent and leaflet fatigue resistance.

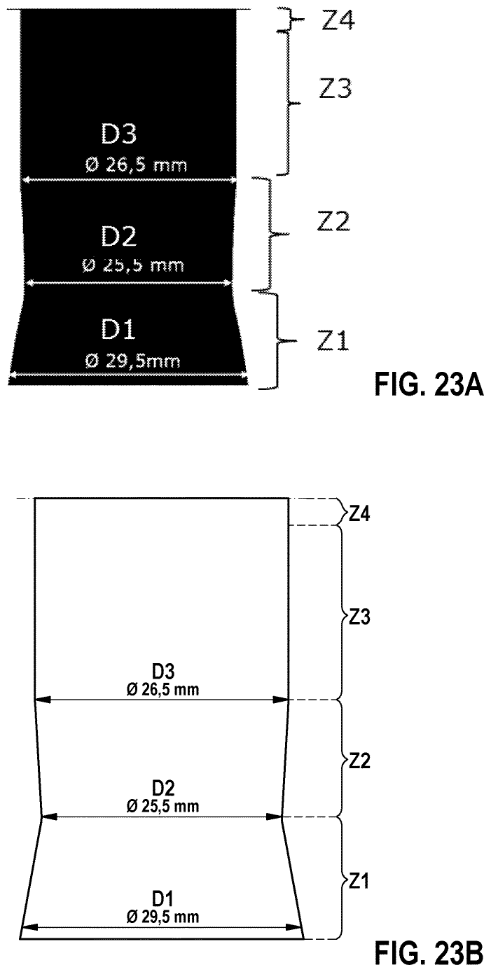

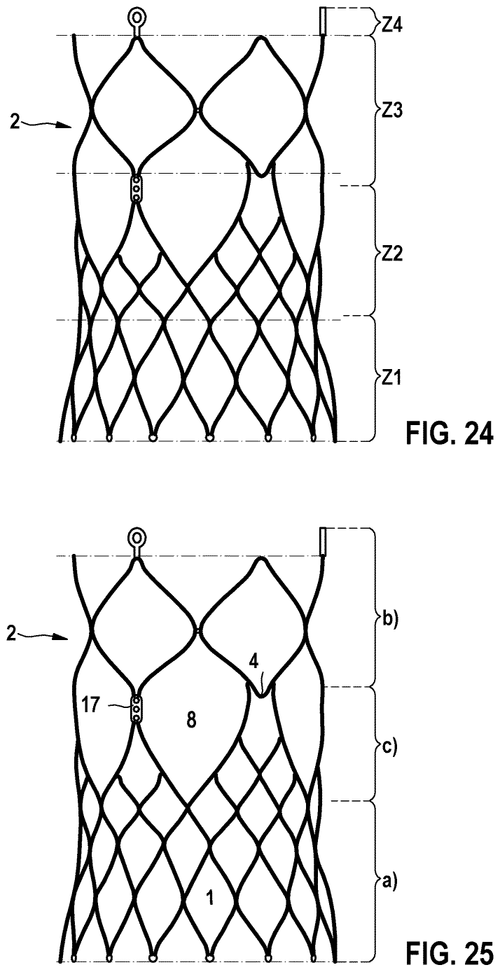

With this in mind, the stent structure in accordance with the present invention and as depicted in FIGS. 23 and 24 is inter alia characterized by:
a conical and convex-shaped inflow section that allows for a stable anchoring of the prosthetic heart valve in the annulus plane while simultaneously reducing any AV node irritation through a specific distribution of the radial force in the inflow section of the stent structure; thus, the radial force (COF, RRF) and the crush resistance of the stent structure, respectively, are adapted to different functional requirements over the entire length of the stent;
a cylindrical and straight outflow section that avoids any undesired contact of the outflow section with the surrounding anatomy;
a conical transition zone between the inflow section and the outflow section having suitably large access to the coronary arteries, e.g., for conceivable coronary intervention;
the said inflow section, transition zone and outflow section all of which are configured to ensure conformability of the stent structure of the prosthesis with the surrounding anatomical structures at the implantation site;
being repositionable/resheathable at least three times without any significant loss of radial force of the stent structure or without any buckling events;
an increased fineness of the inflow structure (e.g. 12, 15 or 19 cells) in order to achieve more adaptation between the stent structure and the annulus plane at the implantation site;
a decreased fineness of the outflow structure (e.g. 3, 6 or 9 cells) in order to achieve more space for coronary access;
a well-adjusted stiffness of the stent over the entire length;
a shortened overall length of the stent structure in order to reduce angular misalignment within the aorta.

Accordingly, the present inventors provide for a prosthetic heart valve that includes a valve assembly (leaflet and skirts) that is well-suited to the stent structure as described herein.

In one embodiment of the invention, the inner and outer skirt material are made from identical material which is porcine pericardium.

In one embodiment of the invention, the inner and outer skirt material may be made from different material such as porcine pericardium and a polymer.

In one embodiment of the invention, the suture material for the leaflet/skirt tissue components is selected from PTFE or UHMWPE. In one embodiment of the invention PTFE is used for the valve subassembly and UHMWPE is used for the sutures of the valve assembly to the stent structure.

The prosthetic heart valve of the present invention is provided for the replacement of an insufficient native aortic heart valve of a subject by minimally invasive transfemoral delivery via a suitable catheter without prior surgical removal of the impaired native aortic valve.

The prosthetic heart valve of the present invention includes a self-expanding stent structure, such as a Nitinol stent, optionally including one or more distal connector elements, an inner skirt element and two or more leaflets, preferably a three-leaflet configuration, all sutured onto the stent structure, and an outer skirt element spreading from the proximal region of the stent, i.e. the inflow section of the stent, up to the prosthesis belly nadir and which is also sutured onto the stent structure together with the said inner skirt element.

The most relevant technical features of the prosthetic heart valve in accordance with the present invention may be summarized as follows: The prosthetic heart valve may be applied in aortic diameters ranging from 20 to 30 mm, but not limited to. The prosthesis includes an inner skirt element and an outer skirt element in order to prevent the prosthesis from paravalvular leakage. The one, two or more leaflets of the prosthesis follow a supra annular valve concept. Following the herein described configurations of the stent structure in connection with the valve assembly of the invention, the entire heart valve prosthesis is fully repositionable and resheathable for at least three times.

The valve leaflets of the invention are designed and sized for high degree of coaptation, reduced stress, low gradient, and high effective orifice area (EOA).

The stitching patterns in accordance with the invention are used to assemble the tissue components safely together (e.g. the leaflets and inner skirt elements) and to allow for a smoother load transfer between the stent structure and the tissue components (leaflets and skirts) in order to increase the prosthetic heart valve's durability.

Specifically, the outer skirt element of the present invention are configured to increase the hydraulic resistance to backflows in diastole phase and to fill potential gaps created by the presence of any geometrical irregularities in the annulus, e.g., due to local calcifications. Moreover, the outer skirt element of the present invention is configured to ensure that the interaction with a delivery system, i.e., the loading/release and resheathing phase, does not impair the short term and long term functionality and safety of the prosthetic heart valve and does not impair or create additional obstruction to the coronary perfusion and for post-implant access.

While certain aspects of the invention have been defined above, protection is claimed for any novel feature or idea described herein and/or illustrated in the drawings, whether or not emphasis has been placed thereon.

The medical implant may be used in the treatment of a stenosis (narrowing) of a cardiac valve and/or a cardiac valve insufficiency and may replace the patient's natural cardiac valve. Each of the at least two leaflets of the medical implant consists of natural tissue or synthetic material and has a first opened position for opening the patient's heart chamber and a second closed position for closing the patient's heart chamber, the at least two leaflets being able to switch between their first and second position in response to the blood flow through the patient's heart. Furthermore, the medical implant may include a compressible and expandable stent-structure so that the medical implant can be delivered to an implantation site using a catheter.

The above expression “narrowing (stenosis) of a cardiac valve and/or cardiac valve insufficiency” may include a functional defect of one or more native cardiac valves of the patient, which is either genetic or has developed. A cardiac defect of this type might affect each of the four heart valves, although the aortic and mitral valves are affected much more often than the right-sided part of the heart (pulmonary and tricuspid valves). The functional defect can result in narrowing (stenosis), inability to close (insufficiency) or a combination of the two (combined vitium). This disclosure relates to a prosthetic heart valve as well as a transcatheter delivered endoprosthesis that includes a prosthetic heart valve and an expandable stent capable of being implant ed transluminal in a patient's body and enlarged radially after being introduced by transcatheter delivery for treating such a heart valve defect. In the case of a stenosis, the native heart valve does not open properly, whereby insufficiency represents the opposite effect showing deficient closing properties. Medical conditions like high blood pressure, inflammatory and infectious processes can lead to such cardiac valve dysfunctions. Either way in most cases the native valves have to be treated by surgery. In this regard, treatment can either include reparation of the diseased heart valve with preservation of the patient's own valve or the valve could be replaced by a mechanical or biological substitute also referred to as heart valve prosthesis. Particularly for aortic heart valves, however, it is frequently necessary to introduce a heart valve replacement.

There is also a desire to improve the medical implant in order to reduce PVL but take the contractibility to the compressed state of the medical implant into account. Further, a simple and cost-effective manufacturing method shall be described for such medical implant.

In particular, the medical implant for transcatheter delivery to an implantation site includes:
an implant component defining a flow passage for guided and/or controlled fluid flow, the flow passage having an inflow end and an outflow end, wherein the medical implant component includes a stent-structure,
the stent-structure having a compressed state and being expandable into a functional state, wherein the stent-structure includes a first stent-structure end, a second stent-structure end, a plurality of struts and a longitudinal axis, wherein the stent-structure includes a plurality of cells, wherein each full cell includes two zig-zag structures formed by a plurality of struts, wherein the vertices of the zig-zag structures are coupled together, wherein each zig-zag structure extends around the full circumference of the stent-structure with regard to the longitudinal axis,
an outer skirt extending at least partly outside the stent-structure, wherein the outer skirt is affixed to the stent-structure using a suture in at least a sealing section and a fold creating section,
wherein in the sealing section the outer skirt is sutured to each strut of at least two directly adjacent zig-zag structures along the full circumference of the stent-structure and
wherein in the fold creating section the outer skirt is sutured to at least part of the struts of one zig-zag structure around the full circumference of the stent-structure and such that the outer skirt forms at least one fold which creates a diameter enlarging structure or fluid redirecting structure by interaction with a fluid flow between the medical implant component and the anatomy when the medical implant is placed at the implantation site.

The medical implant component further surrounds the flow passage and provides support and anchoring of the medical implant at the implantation site, in particular by its stent-structure. The medical implant component may further include a valve assembly, wherein the valve assembly may include the at least two or three leaflets. The valve assembly may further include an inner skirt to which the at least two leaflets are affixed. The valve assembly may extend at least partly within the stent-structure. The inner skirt may extend to the inflow end of the flow passage (or the stent-structure) or the inner skirts end may have a predefined distance from the inflow end of the flow passage (or the stent-structure).

As indicated above the stent-structure has a compressed state and is expandable into a functional state, wherein the outer diameter of the stent-structure is smaller in the compressed state. The stent-structure may be tubular. Furthermore, the stent-structure includes a plurality of cells that define contours of the medical implant component and a longitudinal axis, wherein the stent-structure includes a plurality of cells, wherein each full cell includes two zig-zag structures formed by a plurality of struts. Each half cell may include only one such zig-zag structure. The vertices of the zig-zag structures are coupled together, wherein each zig-zag structure extends around the full circumference of the stent-structure with regard to the longitudinal axis. The stent-structure may be self-expanding or balloon-expandable. The construction of the stent-structure with the cells including zig-zag structures of struts allows to adapt the medical implant to the anatomy of the patient at the implantation site, to affix the further elements of the component, for example a valve assembly, and to affix the outer skirt. Furthermore, the stent-structure absorbs radial and longitudinal forces acting on the medical implant. The stent-structure may further provide a uniform distribution of stress along its plurality of cells. The inner diameter of the stent-structure at the second stent-structure end may be greater than the inner diameter at the first stent-structure end in the functional state. At the first stent-structure end the stent-structure may include a greater diameter than further along the longitudinal axis of the stent-structure into the direction of the second stent-structure end. In such intermediate stent-structure section, the stent-structure may form a belly and a belly nadir.

According to the invention, the PVL is reduced by the outer skirt which extends at least partly outside the stent-structure and/or the component, in one embodiment it is accommodated fully outside the stent-structure, i.e. at its outer surface. The outer skirt is affixed to the stent-structure using a suture. The areas of attachment are at least a sealing section and a fold creating section of the outer skirt. The sealing section provides a stable (firm) and sealed attachment of the outer skirt to the stent-structure. This is realized by suturing the outer skirt to each strut of at least two directly adjacent zig-zag structures along the full circumference of the stent-structure. The sealing section further supports contractibility of the medical implant, in particular with regard to its outer skirt. In the fold creating section the outer skirt is sutured to at least part of the struts of one zig-zag structure in a specific sewing pattern around the full circumference of the stent-structure. The suturing in both sections and the properties of the outer skirt is further adapted such that the outer skirt forms at least one fold which creates a diameter enlarging structure or fluid redirecting structure by interaction with a fluid flow between the medical implant component and the anatomy when the medical implant is placed at the implantation site. The fold creating section provides at least one fold within the outer skirt which develops during compression (i.e. crimping) in a predictable manner. In one embodiment the outside folds protrude towards the anatomy in the functional state thereby augmenting the contact with same. In the other embodiment the fluid is redirected by channels formed by the folds from the direction along the longitudinal direction to a radial direction.

In one embodiment the fold creating section is located further to the second stent-structure end than the sealing section. Thereby, the folded structure directly interacts with the fluid flowing into the reverse direction in order to prevent PVL.

In one embodiment where the fold creating section is located further to the second stent-structure end than the sealing section, the outer skirt is sutured to the stent-structure in the fold creating section to each second strut along the full circumference of the stent-structure. In this embodiment, the outer skirt may include a through-hole, for example a slit or a hole with a circular or half-circular cross section, at a first fold end that is located further to the first stent-structure end than a second fold end. In this configuration, the outer skirt creates conical, slightly bent folds or pleats (bent into the direction perpendicular to the longitudinal direction but having a through hole or slit) in the form of “bagpipes”. After implantation at the implantation site, the reverse blood flow enters the fold at the fold creating section of the outer skirt, flows within the fold and exits the fold at the opposite end through side openings. In the “bag-pipe” skirt configuration blood is not intended to be trapped but redirected. The blood is channeled into the fold at the fold creating section and redirected radially through the side openings thereby reducing PVL. For this embodiment the circumferential length of the outer skirt may be dimensioned such that it equals the stent-structure at a belly level not far from the first stent-structure end and at the inflow level. The height of the skirt may be dimensioned such that it equals the height of the stent-structure between the first stent-structure end and the belly nadir of the stent-structure.

In a further embodiment where the fold creating section is located further to the second stent-structure end than the sealing section, the outer skirt is folded over the outside of the sealing section and is sutured to the stent-structure in the fold creating section to each strut along the full circumference of the stent-structure. In this embodiment, the outer skirt is folded at the first stent-structure end along the entire circumference such that it overlaps the sealing section. In the area of the sealing section, the outer skirt then is a two-layered structure. The section of the outer skirt, which is folded over is then sutured to the stent-structure at its end showing into the outflow direction forming the fold creating section. The outer skirt shows a characteristic cow-slip like shape when finally the medical implant is crimped down to its compressed state. This configures the outer skirt as an enhanced, out-of-round gasket-like structure meant to seal the space between the medical implant and anatomy in the implantation site. In this embodiment the circumferential length of the outer skirt may dimensioned such that it equals the stent-structure at a belly level, if there is a belly nadir, and at the first stent-structure end level. The height of the outer skirt may be longer than the height of the stent-structure between the belly nadir and the first stent-structure end because the outer skirt of this embodiment is supposed to be folded, along the longitudinal axis of the stent-structure, during the assembly of the medical implant. This creates a sort of gasket-like structure (formed as a consequence of the superimposition of at least 2 layers of outer skirt). Moreover, the outer skirt in this embodiment can be forced to have no/negligible axial slack by being constrained through stitching specific spots of the outer layer on the stent-structure to drive the fold consequently to the contraction and the stent foreshortening only circumferentially leading to the cow-slip like shape when finally the medical implant is compressed.

In the above embodiments, the sealing section may be provided by the at least two directly adjacent zig-zag structures being closest (nearest) to the absolute first stent-structure end or one of them forming the absolute first stent-structure end at least partially.

In another embodiment, the fold creating section is located further to the first stent-structure end than the sealing section and wherein the outer skirt is sutured to the stent-structure in the fold creating section to each second strut along the full circumference of the stent-structure. By selectively limiting the stitching in the zig-zag structure at the first stent-structure end, the resulting unconstrained outer skirt surface is folding when the prosthesis is compressed, e.g. by crimping. In this configuration, the skirt assumes a shape that looks similar to a “Hawaiian skirt” (here means a pleated skirt). In the Hawaiian skirt configuration no blood is intended to be trapped. The “Hawaiian skirt” is founded on the concept of intravalvular parietal skirt, wherein the circumferential length of the outer skirt may be dimensioned such that it equals the stent-structure at the belly level and at the level farthest to the first stent-structure end. The height of the outer skirt may be dimensioned such that it equals the height of the stent-structure between the level farthest to the first stent-structure end and the belly nadir. In this embodiment the fold creating section may be provided by the one zig-zag structure being closest (nearest) to the absolute first stent-structure end or forming the absolute first stent-structure end at least partially. The proposed concept pursues actively the strategy of reducing the PVL while minimizing the impact on the overall crimping profile of the medical implant. The invented outer skirt (and the associated sewing pattern) aims at increasing the hydraulic resistance to backflows in diastole phase and to fill potential gaps created by the presence of any geometrical irregularities in the annulus, e.g., due to local calcifications. The outer skirt of this embodiment is configured to ensure a predictable foldability thanks to the specific sewing pattern. It allows therefore to improve the sealing performance without impacting the crimping profile.

In one embodiment, the inner skirt, the outer skirt and leaflets may be constructed of porcine, bovine, equine or other mammalian tissue, such as pericardial tissue, and are sewn, welded, molded or glued together so as to efficiently distribute forces along the leaflets and to the stent-structure. In a particularly preferred embodiment, the inner skirt may include three sections of mammalian tissue that are joined along adjacent edges, so that the tissue folds easily to a collapsed delivery profile without bunching. Alternatively, the inner skirt of the valve assembly and the outer skirt may include a synthetic or polymetric material, such as Dacron, expanded polytetrafluoroethylene (“ePTFE”), or other suitable synthetic graft material. The valve assembly leaflets may be constructed of porcine, bovine, equine or other mammalian tissue, such as pericardial tissue, and are sewn, welded, molded or glued to the skirt so as to efficiently distribute forces along the leaflets and to the stent-structure. The use of synthetic or polymeric materials for the inner skirt in conjunction with mammalian tissue leaflets may offer distinct advantages. In particular, the synthetic material may provide the same structural properties as the mammalian tissue but at reduced thickness, thereby enabling the valve assembly to be collapsed to a smaller delivery profile. Alternatively, the leaflets also may include a synthetic or polymeric material. In accordance with the embodiments of the present invention, the stent-structure includes multiple levels, including a proximal conical inflow section, a constriction region and a flared distal outflow section. Each of the inflow and outflow sections is capable of deforming to a non-circular cross-section to conform to the patient's anatomy, while the constriction region is configured to retain a circular cross-section that preserves proper functioning of the valve assembly.

The stent-structure includes a plurality of cells forming a cell pattern that may vary along the length of the stent-structure to provide a high degree of anchoring and alignment of the valve prosthesis. The cell pattern further is selected to provide a uniform diameter where the commissural joints of the leaflets are attached to the stent-structure, while permitting the inflow and outflow regions to expand to conform to the patient's anatomy. In this manner, optimal functioning of the valve assembly may be obtained even though the stent-structure may be deployed in anatomies having a range of sizes. In addition, the stent-structure resists deformation caused by movement of the heart and enables a functional portion of the valve assembly to be disposed supraannularly to the native valve, with a portion of the valve prosthesis extending into the native valve annulus. The stent-structure may consist of biocompatible metallic material. The stent-structure may include or consist of a shape memory material/alloy, for example Nitinol.

In one embodiment, one suture extends over one zig-zag structure or over at least two zig-zag structures. A suture is a structure that includes a plurality of stitches which are executed one after another using at least one suture thread as suture material. The suture may be formed by a single, continuous thread or by a thread that is cut into a plurality of individual sections, each of which, for example, forming at least one knot. According to this embodiment, the one suture may extend over at least two directly adjacent (along the longitudinal direction) zig-zag structures. Due to suturing, the valve assembly and the outer skirt are reliably attached to the stent-structure without adding too much bulk to the stent-structure, when collapsed. For example, the suture material may be braided multi-filament polyester material. These sutures may have any suitable diameter, typically about 0.07 mm. In order to increase the strength of the connection of valve assembly or outer skirt to the stent-structure, however, the diameter of the multifilament sutures may be increased, for example, up to 0.2 mm. In this way, it is possible that the fundamental bond between valve section of the valve assembly including the at least two leaflets and the struts of the stent-structure exhibits additional stability. On the other hand, the remaining sutures shall be kept as thin as possible to enable collapsing of the medical implant to a small diameter. A common running stitch pattern may be used to obtain said bonding. The stitch pattern may be a locking stitch, a blanket stitch and an Armenian stitch, respectively. Of course, any other suitable stitch pattern (i.e. overlocking stitch, slipstitch or topstitch) may also be possible.

In one embodiment, a second suture overlaps a first suture if both sutures extend over at least two zig-zag structures. Overlapping means in connection with sutures that the second suture crosses the first suture which is produced first at some points, for example at vertices of the respective zig-zag structure. By crossing the first suture the second suture stitches the first suture at the crossing region down thereby affixing the valve assembly and/or the outer skirt more reliably to the stent-structure.

In one embodiment in the fold creating section the suture material is wrapped around those struts without crossing the outer skirt at which it does not affix the outer skirt to the stent-structure. In such section the suture is guided by the respective strut along the structure of the stent-structure in order to continue the suture at the end of strut without affixed outer skirt. Thereby the time of production is reduced.

The outer skirt may be a single piece or may consist of a plurality of pieces which are, for example, sutured together or welded together. The outer skirt may be formed much like a textile piece/cloth having a rectangular form or a slightly curved rectangular form. At its longest, opposite edges, in one embodiment, the outer skirt may include a saw-shaped or scalloped or straight first edge and/or a saw-shaped or scalloped or straight second edge. Using the different types of edges, the outer skirt may be best adapted to the stent-structure structure.

The outer skirt may be fabricated by a 3D-shaping process. With a 3D-shaping process seamless three-dimensional shaped outer skirts can be made. The 3D-shaped outer skirt can have any desired shape and/or the surface of the outer skirt can have any desired structure. The 3D-shaped outer skirt can have an outer shape that enables for a reliable sealing of the medical implant against the surrounding anatomical structures at the implantation site. A sealing can be achieved in an intra-valvar, sub-annular or supra-valvar way, or a combination thereof. The outer skirt may be specifically adapted to the stent-structure (e.g. cylindrical shape). The structure on the surface of the outer skirt may have any shape of a solid figure. The skirt at least partially covering the outer side of the stent-structure or the outer skirt may have a three-dimensional shaped structure.

A multiplicity of possible 3D structures can be used to enable a sealing medical implant against the surrounding anatomical structures at the implantation site. The three-dimensional shaped structure on the surface of the outer skirt may be at least one of a protrusion and/or a recession (valley), preferably
a bubble, sphere, spheroid, ellipsoid, or has a shape generally obtained by cutting such solids with a plane,
a fold or wrinkle
a polyhedron,
a (elongated) channel, turbine-like structure, a wrinkle,
a notch, a recess, a groove,
or combinations thereof.

The three-dimensional shaped structure on the surface of the outer skirt may be the counterform to the anatomical structure sourroundig the medical implant at t implantation site.

The skirt at least partially covering the outer side of the stent-structure or the outer skirt may have a bellow-like structure or may be a pleated skirt (having folds/wrinkels).

The structure on the surface of the outer skirt may have an alternation of peaks and valleys (e.g. at least 30%).

A protrusion may protrude towards the surrounding anatomical structures at the implantation

A recession may protrude away from the surrounding anatomical structures at the implantation site.

The outers structure can have at least one suction cup which can seal the medical implant against the surrounding anatomical structures at the implantation site.

The outer skirt may have at least one notch, recess, gap or channel between the native aortic annulus and the valve prosthesis (after being deployed to improve sealing and decrease PVL leakage yet maintaining a controlled loading profile and controllable folding during crimping).

Furthermore, the 3D-stucture is meant to cover as much as outer skirt surface possible and thereby also most of the sealing hot spots (intra-valvar, sub-annular and supra-valvar). Additionally, the presence of the 3D-structure is meant to increase the hydraulic re-sistance of the medical implant to the blood backflow.

Such 3D shaped outer skirt may facilitate a significant limitation or prevention of the occurrence of any paravalvular leakage through:
(a) increasing the surface area of the outer skirt in a sealing region,
(b) increasing the contact surface of the skirt with an anatomical site surrounding the implant; e.g., within the annular region (but also supra-annular and sub-annular implantation is possible); and
(c) increasing the hydraulic resistance of the medical implant to a blood backflow, especially in the presence of calcific nodules in case of the aortic valve.

The 3D-shaped outer skirt can be applied to any TAVR implant regardless the working principle of the prostheses (i.e. intrannular, suprannular, short stent, long stent). Thus, a 3D-shaped outer skirt is intended to cover most of the variations of patients among a population.

In one embodiment, the outer skirt includes within its suture area a plurality of laser markings that partially penetrate the material of the outer skirt along its thickness. Each laser marking may be used for suturing, wherein the reduced material thickness eases the suturing/sewing process without compromising the structural integrity of the material of the outer skirt (e.g. a biological tissue). Rather than piercing/drilling holes (mechanically or with laser), a laser marking approach has been developed, wherein the spot to be punctured is identified by locally engraving the surface of the material of the outer skirt. Differently from a hole, a laser marking preserves most of the structure of the tissue (e.g. a biological structure) minimizing therefore damages to the structural integrity of the material. As it can be observed, laser markings only partially penetrate the material thickness. By appropriately tuning power and speed of the laser an adequate depth of the laser markings may be identified. Those marks may be sufficiently big to be optically visible at the surface of the outer skirt by the operator during the sewing process and in the meantime sufficiently small to minimally impact the overall structure of the tissue. This may be achieved by appropriately tuning the speed and the power of the laser used to cut the material. This invention improves the assembly process by minimizing manufacturing inaccuracies and manufacturing errors: by assisting every stitch with an optimized minimal indication, the use of laser markings is an effective and suitable way of ensuring design compliance, ensuring quality, and increasing the yield. If the valve assembly consists of a plurality of pieces, the laser markings at the surface of a leaflet or an inner skirt may be used in order to ease suturing of those elements, as well.

Furthermore, with regard to the above usage of a laser to create partially penetrating laser markings (L-ASP), little circles with diameter being, for example smaller than 0.06 mm are used in the CAD software. Afterwards, the CAD file can be converted into the cutting file. For a specified laser currently in use (UNIVERSAL), the first difficulty in manufacturability of laser markings is the full import of little circles into the laser cutter software which form the laser markings after laser operation. On one hand, the circles should be small, so that the laser markings are not too big. On the other hand, very small circles lead to losses during importing the cutting file. There is a trade-off between the amount of circles and their diameter. The more circles on the cutting file are the higher the diameter has to be, in order to be fully imported into the laser cutter software. The circles for the laser markings may have a diameter between 0.01 mm and 0.06 mm. Considering the huge amount of circles, their diameter may be too small. This potentially leads to losses after importing the file. By iteratively increasing the circles diameters at each import, it is possible to identify the minimal diameter required to fully import the circles. Laser markings are a powerful sewing tool and can be used in combination with different topological distributions (of the marks) on the components to be sutured. Examples of those distributions are the double pattern and the alternating pattern, wherein in a double patterned topology the marks are separated with a distance of a pitch length, wherein in an alternating patterned topology the laser markings are separated with a distance of a double pitch length. In the latter case by using an opposing layer having laser markings located shifted with regard to the first layer such that each layer includes a laser marking within a pitch length in an alternating manner.

In one embodiment the suture of the outer skirt and/or a suture of a valve assembly of the medical implant component is overlayed by a protecting stitch structure, wherein the valve assembly extends at least partly within the stent-structure. This means that there are two overlaying suture, the first suture as described above and a second, protecting stitch structure which is sutured over the first suture and hence accommodated over the first suture. Such protecting stitch structure may be used for a suture affixing the outer skirt to the stent-structure but also to a suture affixing a valve assembly to the stent-structure, wherein the valve assembly may include at least two leaflets and, if applicable, an inner skirt. The proposed stitching pattern is instrumental to prevent suture thinning/damaging on the stent-structure and to ensure structural resilience and enhance structural durability of the implant, in particular at critical spots or regions. For the protecting stitch structure, the same thread material may be used as for the first suture. In one embodiment the suture of the outer skirt and/or the suture of the valve assembly is provided in one direction along the longitudinal axis and the protecting stitch structure is provided in the opposite direction relative to the one direction. Further-more, in one embodiment the first suture is formed at least partially by Armenian stitches and/or the protecting stitch structure is formed at least partially by whip stitches. This is because, the higher the radial force and the reduced post-deflection of a stent-structure is, this may lead to accelerated wearing of the suture on the stent-structure struts (so-called suture breakage). Multiple re-sheathing processes of the medical implant might also lead to suture thinning/damaging in some critical area. Further, there may be micro movement of the valve assembly or the outer skirt with regard to the stent-structure which may lead to wearing. Another advantage is that the protecting stitch structure protects the first suture during contraction (e.g. crimping) and forms some sort of buffer for the primary suture.

In another embodiment the suture or the protecting stitch structure includes two knots at a nutcracker eye at a first stent-structure end. With such structure the outer skirt may be fixed to the stent-structure, for example, at a vertex of the first stent-structure end where the struts may converge forming a so-called nutcracker structure with a nutcracker shape. The nutcracker hole is the through-hole in the center of the nutcracker structure. At this structure during suturing two times a loop is created by the thread when it is taken around the strut at the nutcracker hole and a knot is formed by crossing underneath the loop.

In another embodiment the suture or the stitch structure extends through three holes within the outer skirt and/or the valve assembly at a vertex of the stent-structure and wherein the suture or the stitch structure forms a clove-hitch knot. This type of knot is obtained as a variation of a standard whip stitch. In particular, instead of performing a consecutive spiral of suture loops around the stent-structure strut, the suture is crossed-over once (or several times consecutively) through the previous “whip-loop” and around the strut. The tissue shall be punctured only on one side of the stent strut, whereas on the opposite side the suture thread shall be only wrapped around the stent strut, thus pinching the tissue profile in between. This type of knot is inspired by the so called “Clove Hitch”, which is widely used in many non-medical applications (e.g. climbing). It is a self-tightening system when one of the extremities is pulled (for example during crimping or releasing of the implant) , thus inhibiting the mobilization of the suture pattern along the stent-structure. This property makes this type of knot suitable for challenging spots of the assembly of an implant (either BE or SE stent), such as the peaks or extremities of cell pattern, where the likelihood of tissue components mobilization is higher (especially during harsh interactions with the delivery system). The holes within the outer skirt or the valve assembly enhance force/strain distribution. With regard to both above discussed special knot structures the advantage is that the suturing procedure is streamlined in terms of implant manufacturing time and training time this increasing the patter repeatability.

As indicated above the medical implant may be in particular a valve prosthesis and the implantation site is within the patient's heart adjacent to or within a natural heart valve, wherein the medical implant component includes the stent-structure and a/the valve assembly including a at least two leaflets, wherein the valve assembly is affixed to the stent-structure and extends at least partly within the stent-structure.

The medical implant may be implant ed using minimally invasive, catheter based or percutaneous implantation techniques, but implantation using open surgical technique is included, as well.

The above object is, in particular, solved by a manufacturing method for an implant for transcatheter delivery to an implantation site including the following steps:
providing an implant component defining a flow passage for guided and/or controlled fluid flow, the flow passage having an inflow end and an outflow end, wherein the medical implant component includes a stent-structure, wherein the stent-structure has a compressed state and a functional state, wherein the stent-structure includes a first stent-structure end at the inflow end of the flow passage, a second stent-structure end, a plurality of cells that define contours of the medical implant component and a longitudinal axis, wherein each full cell includes two zig-zag structures formed by a plurality of struts, wherein the vertices of the zig-zag structures are coupled together, wherein each zig-zag structure extends around the full circumference of the stent-structure with regard to the longitudinal axis,
providing an outer skirt,
affixing the outer skirt to the stent-structure using a suture in at least a sealing section and a fold creating section such that it extends at least partly outside the stent-structure and
such that in the sealing section the outer skirt is affixed, preferably sutured, to each strut of at least two directly adjacent zig-zag structures along the full circumference of the stent-structure and
such that in the fold creating section the outer skirt is affixed, preferably sutured, to at least part of the struts of one zig-zag structure around the full circumference of the stent-structure and such that the outer skirt forms a fold which creates a diameter enlarging structure or fluid redirecting structure by interaction with the fluid flow when the medical implant is placed at the implantation site.

The above method is simple and cost effective for manufacturing an implant with the above explained advantages. The manufacturing method may include the features as described above with regard to the medical implant. In particular, the manufacturing method may be used for manufacturing a valve prosthesis, wherein the medical implant component includes the stent-structure and a/the valve assembly including at least two of leaflets, wherein the valve assembly is affixed to the stent-structure and extends at least partly within the stent-structure. The valve assembly may include an inner skirt, wherein the at least two leaflets are attached (e.g. sutured) to the inner skirt. In one embodiment, the suturing of the outer skirt may be provided such that the outer skirt is at least partially sutured to the inner skirt.

The invention further includes the embodiments indicated by the following numbered examples.

A. A medical implant for transcatheter delivery to an implantation site including:
an implant component defining a flow passage for guided and/or controlled fluid flow, the flow passage having an inflow end and an outflow end, wherein the medical implant component includes a stent-structure and an outer skirt,
wherein the stent-structure has a compressed state and is expandable into functional state, and wherein the stent-structure includes a first stent-structure end, second stent-structure end opposite the first stent-structure end, a plurality of cells and a longitudinal axis,
wherein each full cell includes two zig-zag structures formed by a plurality of struts, wherein the vertices of the zig-zag structures are coupled together, wherein each zig-zag structure extends around the full circumference of the stent-structure with regard to the longitudinal axis,
an outer skirt extending at least partly outside the stent-structure, wherein the outer skirt is affixed to the stent-structure and/or to an inner skirt in at least a sealing section and a fold creating section,
wherein in the sealing section the outer skirt is affixed to each strut of at least two directly adjacent zig-zag structures along the full circumference of the stent-structure and/or to the inner skirt, and
wherein in the fold creating section the outer skirt is affixed to at least part of the struts of one zig-zag structure around the full circumference of the stent-structure and/or to the inner skirt such that the outer skirt forms a fold which creates a diameter enlarging structure or fluid redirecting structure by interaction with a fluid flow between the medical implant component and the anatomy when the medical implant is placed at the implantation site.

B. The medical implant of embodiment A, wherein the fold creating section is located further to the second stent-structure end than the sealing section.

C. The medical implant of embodiment B, wherein the outer skirt is sutured to the stent-structure in the fold creating section to each second strut along the full circumference of the stent-structure.

D. The medical implant of embodiment C, wherein the outer skirt includes a through-hole, for example a slit or a hole with a circular or half-circular cross section, at a first fold end that is located further to the first stent-structure end than a second fold end.

E. The medical implant of embodiment B, wherein the outer skirt is folded over the outside of the sealing section and is sutured to the stent-structure in the fold creating section to each strut along the full circumference of the stent-structure.

F. The medical implant of embodiment A, wherein the fold creating section is located further to the first stent-structure end than the sealing section and wherein the outer skirt is sutured to the stent-structure in the fold creating section to each second strut along the full circumference of the stent-structure.

G. The medical implant of any of the previous embodiments, wherein one suture extends over one zig-zag structure or over at least two zig-zag structures.

H. The medical implant of embodiment G, wherein a second suture overlaps a first suture if both sutures extend over at least two zig-zag structures.

I. The medical implant of any of the embodiments C to D and F to H, wherein in the fold creating section the suture material is wrapped around those struts without crossing the outer skirt at which it does not affix the outer skirt to the stent-structure.

J. The medical implant of any of the previous embodiments, wherein the outer skirt includes a saw-shaped or scalloped or straight first edge and/or a saw-shaped or scalloped or straight second edge.

K. A medical implant for transcatheter delivery to an implantation site including:
an implant component defining a flow passage for guided and/or controlled fluid flow, the flow passage having an inflow end and an outflow end, wherein the medical implant component includes a stent-structure and an outer skirt and a valve assembly,
wherein the stent-structure has a compressed state and is expandable into functional state, and wherein the stent-structure includes a first stent-structure end, second stent-structure end opposite the first stent-structure end, a plurality of cells and a longitudinal axis,
an outer skirt extending at least partly outside the stent-structure, wherein the outer skirt is affixed to the stent-structure at a suture area using a suture.

L. The medical implant of any of the embodiments A to J, wherein the outer skirt, the inner skirt and/or the leaflets include within their suture areas a plurality of laser markings that partially penetrate the material of which they are made along its thickness or wherein the outer skirt, the inner skirt and/or the leaflets include within their suture areas a plurality of laser holes that fully penetrate the material of which they are made along its thickness.

M. The medical implant of embodiment K or L, wherein the laser marking is optically visible at the outer surface of the outer skirt.

N. A medical implant for transcatheter delivery to an implantation site including:
an implant component defining a flow passage for guided and/or controlled fluid flow, the flow passage having an inflow end and an outflow end, wherein the medical implant component includes a stent-structure, an outer skirt and a valve assembly,
wherein the stent-structure has a compressed state and is expandable into functional state, and wherein the stent-structure includes a first stent-structure end, second stent-structure end opposite the first stent-structure end, a plurality of cells and a longitudinal axis,
an outer skirt extending at least partly outside the stent-structure, wherein the outer skirt is affixed to the stent-structure using a suture,
wherein a suture of the outer skirt and/or a suture of a valve assembly of the medical implant component is overlayed by a protecting stitch structure, wherein the valve assembly extends at least partly within the stent-structure.

O. The medical implant of any of the embodiments A to M, wherein the suture of the outer skirt and/or a suture of a valve assembly of the medical implant component is overlayed by a protecting stitch structure, wherein the valve assembly extends at least partly within the stent-structure.

P The medical implant of embodiment O, wherein the suture of the outer skirt and/or the suture of the valve assembly is provided in one direction along the longitudinal axis and the protecting stitch structure is provided in the same or opposite direction relative to the one direction.

Q The medical implant of any of the previous embodiments A to P, wherein the suture is formed at least partially by Armenian stitches and/or the protecting stitch structure is formed at least partially by whip stitches.

R. A medical implant for transcatheter delivery to an implantation site including:
an implant component defining a flow passage for guided and/or controlled fluid flow, the flow passage having an inflow end and an outflow end, wherein the medical implant component includes a stent-structure and an outer skirt,
wherein the stent-structure has a compressed state and is expandable into functional state, and wherein the stent-structure includes a first stent-structure end, second stent-structure end opposite the first stent-structure end, a plurality of cells and a longitudinal axis,
an outer skirt extending at least partly outside the stent-structure, wherein the outer skirt is affixed to the stent-structure using a suture, wherein the suture or the stitch structure includes two knots at a nutcracker eye at a first stent-structure end.

S. The medical implant of any of the embodiments A to Q, wherein the suture structure or the stitch structure includes two knots at a nutcracker eye at a first stent-structure end.

T. The medical implant of any of the embodiments A to S, wherein the suture structure or the stitch structure extends through holes within the outer skirt and/or the valve assembly at a vertex of the stent-structure and wherein the suture or the stitch structure forms a clove-hitch knot.

U. The medical implant of any of the embodiments of claim A to T, wherein the medical implant is a heart valve prosthesis and the implantation site is within the patient's heart adjacent to or within a natural heart valve, wherein the medical implant component includes the stent-structure and a/the valve assembly including a at least two leaflets, wherein the valve assembly is affixed to the stent-structure and extends at least partly within the stent-structure.

V. A manufacturing method for an implant for transcatheter delivery to an implantation site including the following steps:
providing an implant component defining a flow passage for guided and/or controlled fluid flow, the flow passage having an inflow end and an outflow end, wherein the medical implant component includes a stent-structure and an outer skirt, wherein the stent-structure has a compressed state and is expandable into functional state, and wherein the stent-structure includes a first stent-structure end, second stent-structure end opposite the first stent-structure end, a plurality of cells and a longitudinal axis, wherein each full cell includes two zig-zag structures formed by a plurality of struts, wherein the vertices of the zig-zag structures are coupled together, wherein each zig-zag structure extends around the full circumference of the stent-structure with regard to the longitudinal axis,
providing an outer skirt,
affixing the outer skirt to the stent-structure using a suture in at least a sealing section and a fold creating section such that it extends at least partly outside the stent-structure and
such that in the sealing section the outer skirt is sutured to each strut of at least two directly adjacent zig-zag structures along the full circumference of the stent-structure and
such that in the fold creating section the outer skirt is sutured to at least part of the struts of one zig-zag structure around the full circumference of the stent-structure and such that the outer skirt forms a fold which creates a diameter enlarging structure or fluid redirecting structure by interaction with the fluid flow when the medical implant is placed at the implantation site.

W. The method of embodiment V, wherein the fold creating section is located further to the second stent-structure end than the sealing section.

X. The method of embodiment W, wherein the outer skirt is sutured to the stent-structure in the fold creating section to each second strut along the full circumference of the stent-structure.

Y. The method of embodiment X, wherein the outer skirt includes a through-hole, for example a slit or a hole with a circular or half-circular cross section, at a first fold end that is located further to the first stent-structure end than a second fold end.

Z. The method of embodiment W, wherein the outer skirt is folded over the outside of the sealing section and is sutured to the stent-structure in the fold creating section to each strut along the full circumference of the stent-structure.

AA. The method of embodiments V, wherein the fold creating section is located further to the first stent-structure end than the sealing section and wherein the outer skirt is sutured to the stent-structure in the fold creating section to each second strut along the full circumference of the stent-structure.

BB. The method of any of the embodiments V to AA, wherein one suture is sutured such that it extends over one zig-zag structure or over at least two zig-zag structures.

CC. The method of embodiment BB, wherein a second suture is sutured after a first suture such that it overlaps the first suture if both sutures extend over at least two zig-zag structures.

DD. The method of any of the embodiments X to Y and AA to CC, wherein during suturing in the fold creating section the suture material is wrapped around those struts without crossing the outer skirt at which it does not affix the outer skirt to the stent-structure.

EE. The method of any of the embodiments V to DD, wherein the outer skirt is provided including a saw-shaped or scalloped or straight first edge and/or a saw-shaped or scalloped or straight second edge.

FF. The method of any of the embodiments V to EE, wherein in a suture area of the outer skirt, the inner skirt and/or the leaflets a plurality of laser markings are created that partially penetrate the material of the outer skirt along its thickness or wherein in a suture area of the outer skirt the inner skirt and/or the leaflets a plurality of laser holes are created that fully penetrate the material of the outer skirt along its thickness.

GG. The method of embodiment FF, wherein the laser marking is optically visible at the outer surface of the outer skirt.

HH. The method of any of the embodiments V to GG, wherein after suturing the suture of the outer skirt and/or after suturing a suture of a valve assembly of the medical implant component an overlaying protecting stitch structure is provided over the respective suture, wherein the valve assembly extends at least partly within the stent-structure.

II. The medical implant of embodiment HH, wherein the suture of the outer skirt and/or the suture of the valve assembly is provided in one direction along the longitudinal axis and the protecting stitch structure is provided in the opposite direction relative to the one direction.

JJ. The medical implant of any of the embodiments V to II, wherein the suture is formed at least partially by Armenian stitches and/or the protecting stitch structure is formed at least partially by whip stitches.

KK. The method of any of the embodiments V to II, wherein the suture or the stitch structure is sutured such that it forms two knots at a nutcracker eye at a first stent-structure end.

LL. The method of any of the embodiments V to KK, wherein the suture or the stitch structure is sutured such that it extends through three holes within the outer skirt and/or the valve assembly at a vertex of the stent-structure and such that the suture or the stitch structure forms a clove-hitch knot.

MM. The method of any of the embodiments V to LL, wherein the medical implant is a heart valve prosthesis, wherein the medical implant component includes the stent-structure and a/the valve assembly including at least two of leaflets, wherein the valve assembly is affixed to the stent-structure and extends at least partly within the stent-structure.

NN. A manufacturing method for a medical implant, preferably a transcatheter deliverable endoprosthesis or a prosthetic heart valve, including the following steps:
providing a stent-structure (1), one or more skirts (5, 6, 32) and a valve assembly (3) having one or more leaflets (31);
optionally creating a plurality of laser markings (45) that partially penetrate the material of the outer skirt, the inner skirt and/or the one or more leaflets in a suture area of the outer skirt, the inner skirt and/or the one or more leaflets, optionally creating a plurality of laser holes that fully penetrate the material of the outer skirt, the inner skirt and/or the one or more leaflets in the suture area of the outer skirt, the inner skirt and/or the one or more leaflets;
wherein the stent-structure (1) has a longitudinal axis (18), a circumference, a first stent-structure end (11) and a second stent-structure end (12) being opposite to the first stent-structure end (11), and wherein the stent-structure (1) surrounds an inner volume (8) and wherein the stent-structure (1) has a plurality of struts (4) forming cells; and
at least partially covering the stent-structure (1) with the one or more skirts (5, 6, 32), such that one skirt (5, 6) of the one or more skirts (5, 6, 32) at least partially covers an outer side of the stent-structure (1); and
arranging the valve assembly (3) within the inner volume (8) of the stent-structure and affixing the valve assembly (3) to the one or more skirts (5, 6, 32) and/or to the stent-structure; and
affixing the one or more skirts (5, 6, 32) to each other, to the stent-structure and/or to the valve assembly, such that the one skirt (5, 6) of the one or more skirts (5, 6, 32) at least partially covering the outer side of the stent-structure extends at least partly outside the stent-structure (1), preferably extends radially outward the circumference of the stent-structure (1).

OO. A medical implant (2), preferably a transcatheter deliverable endoprosthesis or a prosthetic heart valve, including a stent-structure (1), one or more skirts (5, 6, 32) and optionally a valve assembly (3);
wherein the stent-structure (1) has a longitudinal axis (18), a circumference, a first stent-structure end (11) and a second stent-structure end (12) being opposite to the first stent-structure end (11), and wherein the stent-structure (1) surrounds an inner volume (8) and wherein the stent-structure (1) has a plurality of struts (4) forming cells;
wherein one skirt (5, 6) of the one or more skirts (5, 6, 32) at least partially covers an outer side of the stent-structure (1) and extends at least partly outside the stent-structure (1), preferably extends radially outward the circumference of the stent-structure (1), and/or in that the medical implant (2) includes the valve assembly (3) arranged within the inner volume (8) of the stent-structure.

PP. A medical implant (2) according to embodiment OO, wherein the stent-structure (1) has a compressed state and is expandable into a functional state.

QQ. A medical implant (2) according to embodiment PP, characterized in that the skirt (5, 6) at least partially covering the outer side of the stent-structure (1) has protrusions extending radially outward the circumference of the stent-structure (1), at least in the functional state.

RR. A medical implant (2) according to any of the preceding embodiments, characterized in that the medical implant (2) includes only one skirt (6) at least partially covering the outer side of the stent-structure (1) which is at least partially or fully wrapped around the first stent-structure end (11) and at least partially covers an inner side of the stent-structure (1).

SS. A medical implant (2) according to embodiment RR, characterized in that the one skirt (6) at least partially covering the outer side of the stent-structure (1) being at least partially or fully wrapped around the first stent-structure end (11) and is at least partially affixed, preferably sutured, glued and/or welded, to itself and/or to at least some of the plurality of struts (4).

TT. A medical implant (2) according to any of embodiments OO to QQ, characterized in that the skirt (5, 6) at least partially covering the outer side of the stent-structure (1) is an outer skirt (5) and optionally the medical implant (2) further includes an inner skirt (32) covering at least partially the inner side of the stent-structure (1).

UU. A medical implant (2) according to embodiment TT, characterized in that the outer skirt (5) and the inner skirt (32) are at least partially affixed, preferably sutured, glued and/or welded, to each other and/or the outer skirt (5) is at least partially affixed, preferably sutured, glued and/or welded, to at least some of the plurality of struts (4).

VV. A medical implant (2) according to embodiment TT or UU, characterized in that the inner skirt (32) does not cover the first stent-structure end (11) and/or does not extend to the first stent-structure end (11).

WW. A medical implant (2) according to embodiment TT or UU, characterized in that the outer skirt (5) covers the first stent-structure end (11) and/or extends to the first stent-structure end (11).

XX. A medical implant (2) according to any of embodiments 46 to 48, characterized in that the outer skirt (5) is affixed to each strut (4) or each second strut (42) of the stent-structure
(1) at the first stent-structure end (11) along the full circumference.

YY. A medical implant (2) according to any of embodiments TTto XX, characterized in that the outer skirt (5) has a first outer skirt edge (51) and a second outer skirt edge (52) and is folded between the first outer skirt edge (51) and the second outer skirt edge (52) and thereby forming a hook like or loop like structure (7).

ZZ. A medical implant (2) according to embodiment YY, characterized in that the hook like or loop like structure (7) is situated at the first stent-structure end (11).

AAA. A medical implant (2) according to any of embodiments UU to ZZ, characterized in that the first outer skirt edge (51) is affixed to the inner skirt (32) and/or the stent structure (1) and/or to the second outer skirt edge (52) or to the hook like or loop like structure (7).

BBB. A medical implant (2) according to any of embodiments UU to AAA, characterized in that the second outer skirt edge (52) is affixed to the inner skirt (32) and/or the stent-structure (1).

CCC. A medical implant (2) according to any of embodiments YY to BBB, characterized in that the first outer skirt edge (51) is affixed to the inner skirt (32) and the stent-structure (1) and the second outer skirt edge (52) is affixed to the inner skirt (32) and the stent-structure (1).

DDD. A medical implant (2) according to any of embodiments 46 to 54, characterized in that the inner skirt (32) covers the inner side of the stent-structure (1) in the full circumference of the stent-structure (1) and/or the outer skirt (5) covers the outer side of the stent-structure (1) in the full circumference of the stent-structure (1)

EEE. A medical implant (2) according to any of the preceding embodiments, characterized in that the skirt (5, 6) at least partially covering the outer side of the stent-structure (1) or the outer skirt (5) has one or more pleats (70) or is a pleated skirt having pleats (70).

FFF. A medical implant (2) according to embodiment EEE, characterized in that the pleats (70) extend in the direction from the first stent-structure end (11) to the second stent-structure end (12) or the pleats (70) extend in the direction of the longitudinal axis (18) or the pleats (70) are bent with an angle of less than 30°, preferably less than 20°, with respect to the longitudinal axis (18).

GGG. A medical implant (2) according to embodiment EEE or FFF, characterized in that the pleats (70) have a horn-like or bagpipe shape.

HHH. A medical implant (2) according to embodiment GGG, characterized in that at least some or each of the horn-like or bagpipe shaped pleats form a top opening.

III. A medical implant (2) according to embodiment HHH, characterized in that at least some or each of the horn-like or bagpipe shaped pleats having a top opening additionally have a through hole 63, slit or side opening.

JJJ. A medical implant (2) according to any of the preceding embodiments, characterized in that at least a part of the stent-structure (1), at least a part of the at least one skirt (5, 6, 32) and optionally the valve assembly (3) form an implant component, wherein the medical implant component defines a flow passage for guided and/or controlled fluid flow and the flow passage has an inflow end and an outflow end being opposite the inflow end.

KKK. A medical implant (2) according to embodiment JJJ, characterized in that the second stent-structure end (12) is spaced apart from the outflow end of the flow passage.

LLL. A medical implant (2) according to embodiment III, characterized in that the first stent-structure end (11) is spaced apart from the inflow end of the flow passage.

MMM. A medical implant (2) according to any of the preceding embodiments, characterized in that the plurality of struts (4) form at least two zig-zag structures, meander structures or wave line structures, wherein each zig-zag structure, meander structure or wave line structure extends around the full circumference of the stent-structure (1), and wherein at least two zig-zag structures, meander structures or wave line structures are connected so as to form closed cells (202, 203, 204, 205), preferably rhomboid cells.

NNN. A medical implant (2) according to any of the preceding embodiments, characterized in that the valve assembly (3) includes one or more, preferably two or three, leaflets (31).

OOO. A medical implant (2) according to any of the preceding embodiments, characterized in that the one or more skirts (5, 6, 32) and/or the one or more leaflets (31) include one or more laser markings (45) or laser holes.

PPP. A medical implant (2) according to embodiment NNN or OOO, characterized in that the laser markings (45) are optically visible from one side or from both sides of the material of which the one or more skirts or the one or more leaflets (31) of the valve assembly (3) are made.

QQQ. A medical implant (2) to any of the preceding embodiments, characterized in that the one or more skirts (5, 6, 32), preferably the inner skirt (32) and/or the outer skirt (5), and/or the one or more leaflets (31) of the valve assembly (3) are made of a biological tissue, preferably pericardial tissue.

RRR. Method for creating a plurality of laser markings that partially penetrate a biological material, preferably a biological tissue, or creating a plurality of laser holes that fully penetrate the biological material, preferably the biological tissue, having the following process steps:
providing the biological material, preferably the biological tissue, and
providing a laser, and treating the biological material, preferably the biological tissue such that a plurality of laser markings that partially penetrate the biological material, preferably the biological tissue, or a plurality of laser holes that fully penetrate the biological material, preferably the biological tissue, are formed.

Preferred embodiments of the invention will now be discussed with respect to experiments and drawings. Broader aspects of the invention will be understood by artisans in view of the general knowledge in the art and the description of the experiments that follows.

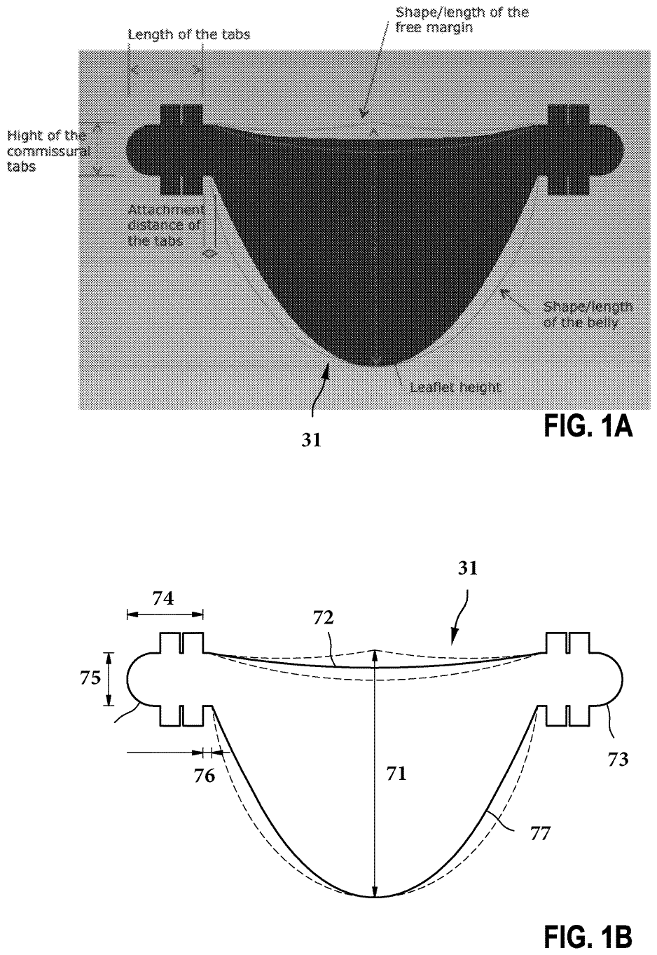

FIGS. 1A, 1B, 2A, 2B and 3 show embodiments of leaflets, which can be used in a heart valve assembly according to the invention. The leaflets may be made of pericardial tissue.

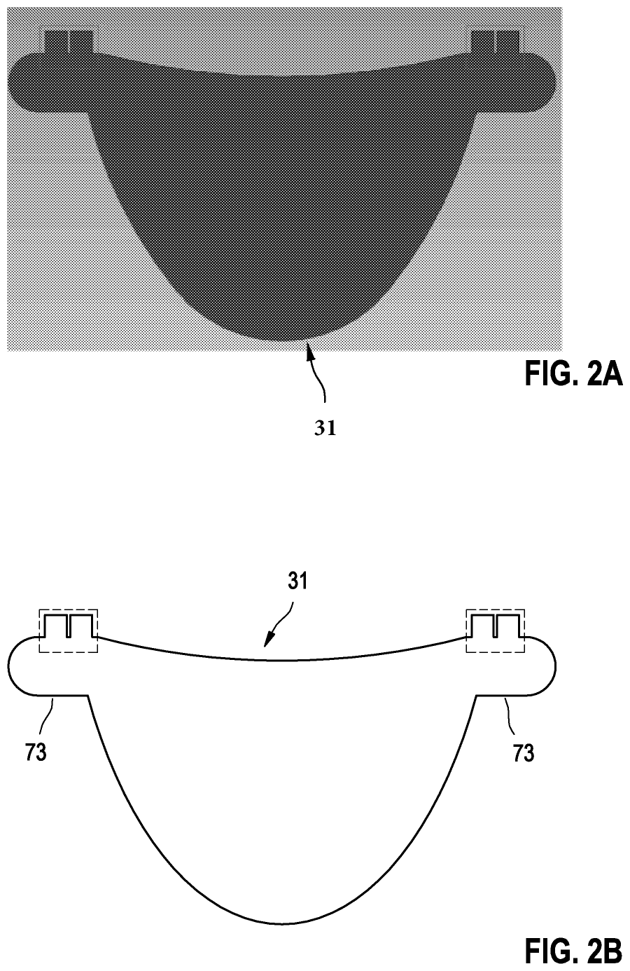

FIGS. 1A and 2B depict the general configuration of the one or more leaflets 31 in accordance with the first aspect of the present invention. In black colour one respective embodiment of the dimensions of the one or more leaflets of the invention is shown in FIG. 1A. In dashed lines, and, e.g., with arrowheads, possible dimensional changes of the above embodiment in black colour are depicted. Based on the individual requirements of a patient, the one or more leaflets of the invention may have to be adapted to the stent-structure of the disclosed prosthetic heart valve that is intended to be implant ed. In order to do so, the one or more leaflet may have to be varied in its configuration as shown by the said dashed lines that may include arrowheads. The leaflet 31 has a belly, a leaflet height 71, a leaflet width 72 (free length of the margin) and a belly length 77. The leaflet has two commissural tabs 73 each having a length 74 and a height 75 and an attachment distance 76 to the belly leaflet. Each of the commissural tabs 73 has superior and inferior alignment tabs. The dashed lines show the possible dimensional changes of the leaflet.

FIGS. 2A and 2B depict the general configuration of the one or more leaflets 31 in accordance with the second aspect of the present invention. In black colour one respective embodiment of the dimensions of the one or more leaflets of the invention is shown in FIG. 2A. The rectangles in the upper portion on the left and right hand side emphasize the superior alignment tabs. In comparison to FIG. 1A, in the second aspect of the invention the inferior alignment tabs of the leaflets are removed from the lower part of the commissural tabs 73. This exemplary configuration of the one or more leaflets of the second aspect of the present invention may be also used in a laser-aided sewing process (L-ASP). If used in L-ASP, in another embodiment of the second aspect of the invention also the superior tabs can be removed as they are not necessary in case of L-ASP as disclosed herein.

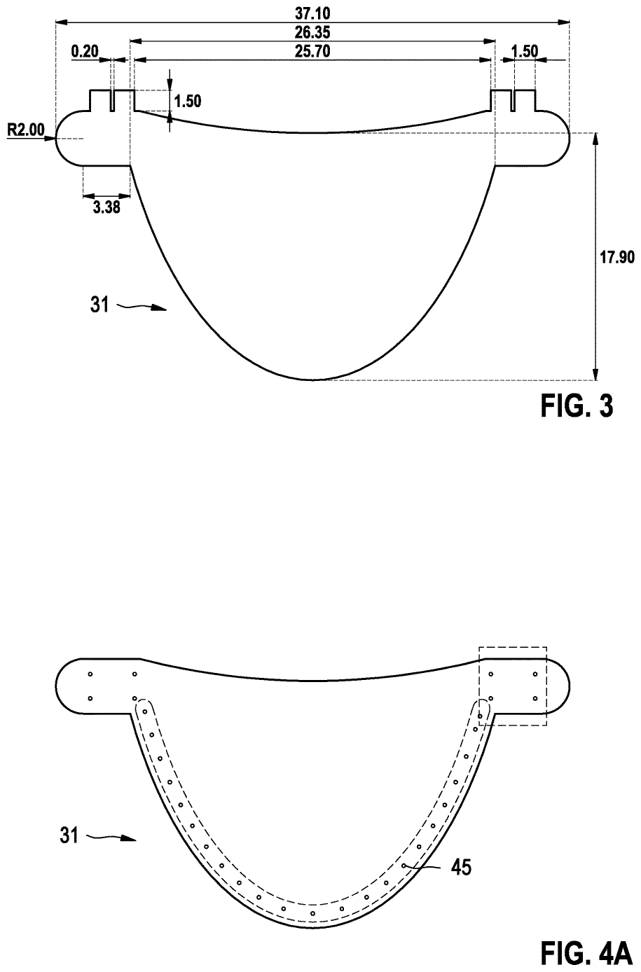

FIG. 3 depicts a schematic general configuration of the one or more leaflets 31 also in accordance with the second aspect of the present invention. In addition, FIG. 3 shows exemplary dimensions of the said configuration in [mm]. In another embodiment of the second aspect of the present invention, this exemplary configuration of the one or more leaflets may be also useful in L-ASP. If used in L-ASP, in another embodiment of the second aspect of the invention also the superior tabs can be removed as they are not necessary in case of L-ASP as disclosed herein.

FIG. 3 shows exemplary dimensions of the leaflet in mm. The shown leaflet 31 has a leaflet height of 17.90 mm and a leaflet width (free length of the margin) of 25.70 mm. The leaflet has two commissural tabs 73 with superior alignment tabs. Depending on the patient's anatomy the dimensions of the leaflet(s) may be adapted to the anatomical size of the patient. However, the relative proportions as shown in FIG. 3 may be kept.

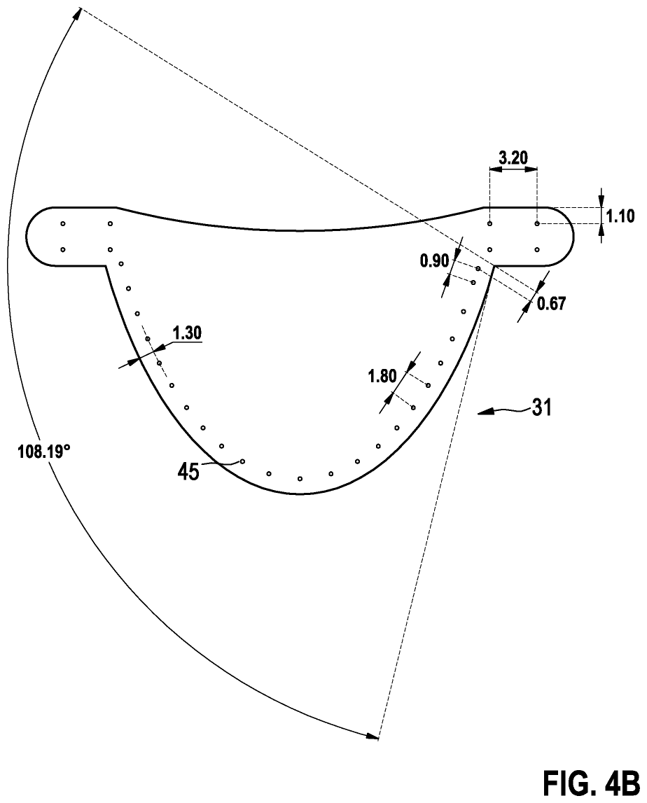

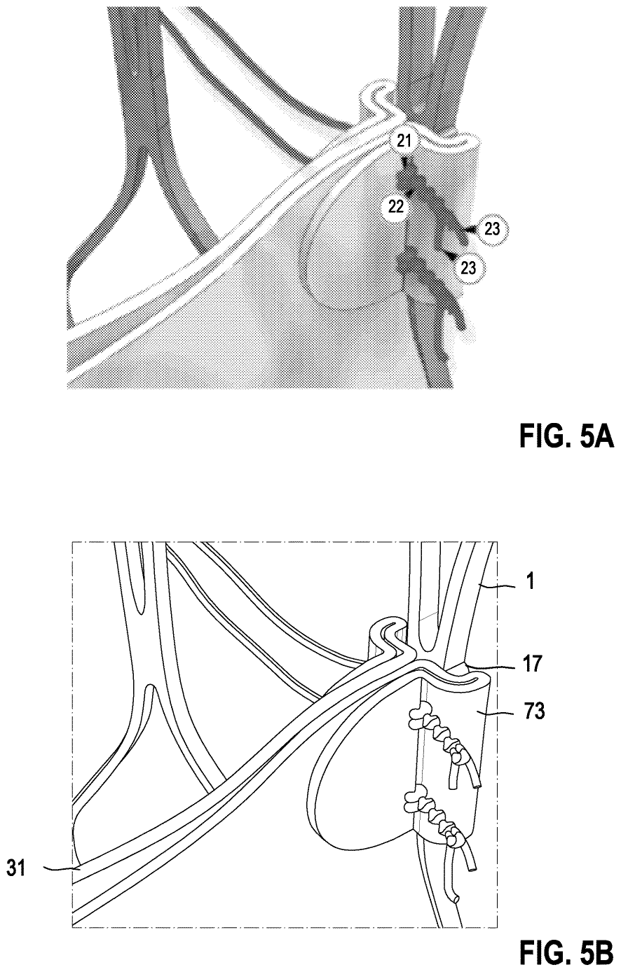

FIGS. 4A and 4B depict a schematic general configuration of the one or more leaflets 31 in accordance with the third aspect of the present invention. In addition, FIG. 4 shows various laser markings where a laser needs to laser a marking or a hole for the sutures in order to fixate the leaflet on the stent structure and to fixate an inner skirt element on the belly portion of the one or more leaflets of the third aspect of the invention. This exemplary configuration of the one or more leaflets of the third aspect of the present invention is particularly suitable for the use in L-ASP. Accordingly, due to the laser-sewing pattern with the laser markings or laser holes such a leaflet configuration does not require any alignment tabs on the commissural tabs. The laser markings or laser holes itself as emphasized in a rectangle on the right are to be used for the alignment and folding of the commissural tabs via respective sutures. See hereto also FIG. 5 below. With this context, “laser marking” denotes only a “laser scratching” on the top of the surface of the pericardium in order to remove only a minimum amount of material. Thus, no through holes are generated by the laser in this case, but under the microscope a skilled person performing the sewing of the skirt/leaflet components can see these markings and may easier follow a given sewing pattern. The sewing process itself is thereby regular. In contrast, a “laser hole” denotes a complete through hole through the pericardium in order to obtain the channels for later sewing readily without the use of any needles. The laser markings are shown in a bended emphasis in the belly portion of the exemplary leaflet of FIG. 4 and are to be used for the fixation via sutures between an inner skirt element and a leaflet. FIGS. 4A and 4B show exemplary dimensions of the leaflet 31 in mm. As shown in FIG. 4B the laser markings 45 or laser holes at the belly of the leaflet may have a distance to each other of between 0.90 mm and 1.80 mm. The laser markings 45 or laser holes at the belly of the leaflet may have a distance to the edge of the belly of 1.30 mm. The laser markings or laser holes at each of the commissural tabs 73 may have a distance to each other of 3.20 mm.

FIGS. 5A and 5B depict an exemplary schematic folding and suturing technique in accordance with the present invention for the one or more leaflets in accordance with the third aspect of the present invention. This exemplary folding and suturing technique of the one or more leaflets in accordance with the third aspect of the present invention is particularly suitable for the use in L-ASP. In FIG. 5B it can be seen that two leaflets 31 are attached to one commissural post 17 of the stent-structure 1, preferably by suturing the commissural tab 73 of each leaflet 31 with sutures 21, 22, 23 to the commissural post 17 of the stent-structure 1.

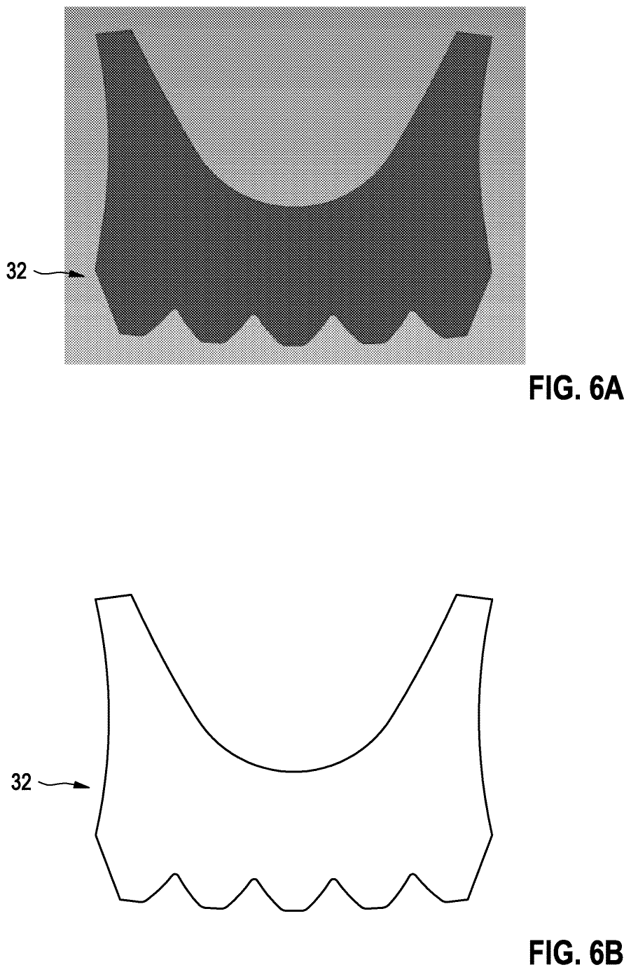

FIGS. 6A and 6B depict the general configuration of the one or more inner skirt 32 elements in accordance with the fourth aspect of the present invention. In black colour one respective embodiment of the inner skirt elements is shown with its exemplary dimensions and contours. The lower proximal scallop portion of the inner skirt element is configured such that any bag formation during systole and diastole of a respective prosthetic heart valve implementing the said inner skirt element is avoided.

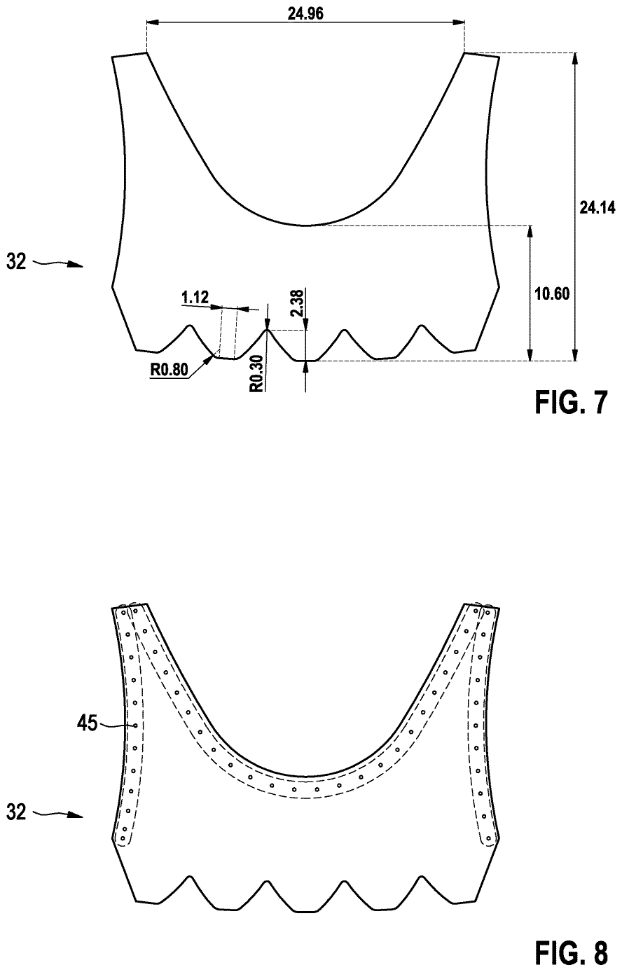

FIG. 7 depicts the general contours of the one or more inner skirt 32 elements in accordance with the fourth aspect of the present invention. In addition, exemplary dimensions of the said one or more inner skirt elements in accordance with the fourth aspect of the present invention are shown; particularly the exemplary dimensions of the scalloped proximal portion. As can be derived from FIG. 7, the scalloped portion is configured such that any bag formation during systole and diastole of a respective prosthetic heart valve implementing the said inner skirt element is avoided. FIG. 7 shows exemplary dimensions of the inner skirt 32 in mm. The inner skirt 32 has a inner skirt height of 24.14 mm.

FIG. 8 depicts the general configuration of the one or more inner skirt 32 elements in accordance with the fifth aspect of the present invention and its contours. The lower proximal scallop portion of the inner skirt element is configured such that any bag formation during systole and diastole of a respective prosthetic heart valve implementing the said inner skirt element is avoided. In addition, in the fifth aspect of the invention the one or more inner skirt elements include laser markings 45 or laser holes for subsequent suturing of the inner skirt elements to another inner skirt element and to a leaflet portion of a prosthetic heart valve. That is, the laser marking or the laser hole on the left and right hand side are used for subsequent suturing of the inner skirt element to a respective other inner skirt element and the laser markings or laser holes in the middle portion highlighted with bended emphasis are used for subsequent suturing of the inner skirt element to a respective leaflet portion. Accordingly, the one or more inner skirt elements in accordance with the fifth aspect of the present invention are particularly useful for L-ASP, which simplify the overall assembly process of a prosthetic heart valve in accordance with the present invention.

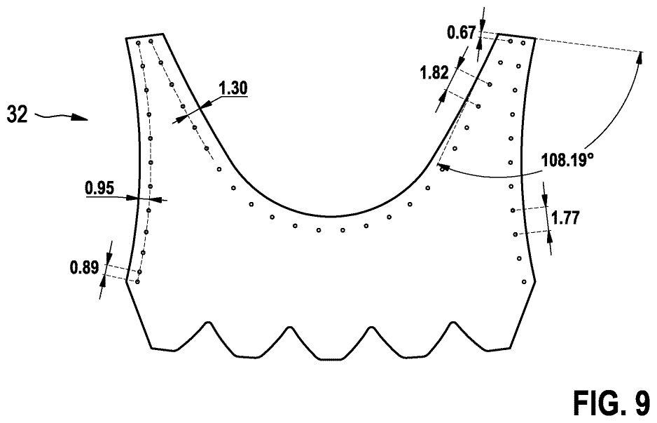

FIG. 9 depicts the general configuration of the one or more inner skirt 32 elements in accordance with the fifth aspect of the present invention and exemplary dimensions and angles thereof. The lower proximal scallop portion of the inner skirt element is configured such that any bag formation during systole and diastole of a respective prosthetic heart valve implementing the said inner skirt element is avoided. In addition, in the fifth aspect of the invention the one or more inner skirt elements include laser markings or laser holes for subsequent suturing of the inner skirt elements to another inner skirt element and to a leaflet portion of a prosthetic heart valve. See hereto also FIG. 8 above. As shown in FIG. 9 the laser markings 45 or laser holes may have a distance to each other of between 0.89 mm and 1.82 mm. The laser markings 45 or laser holes may have a distance to the edge of the inner skirt 32 of between 0.92 mm and 1.30 mm.

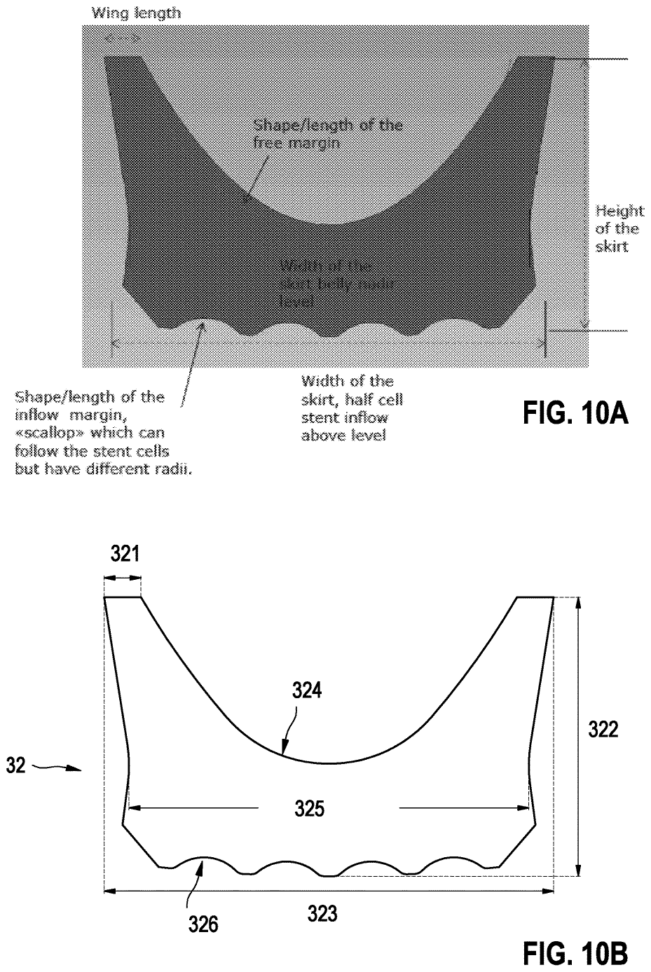

FIGS. 10A and 10B depict the general configuration of the inner skirt 32 element in accordance with the fourth aspect of the present invention. In black colour one respective embodiment of the dimensions of the inner skirt element of the invention in short version is shown in FIG. 10, which means that the lower proximal end portion of the inner skirt element remains one half cell above the stent inflow section. In dashed lines, and, e.g., with arrowheads, possible dimensional changes of the above embodiment in black colour are depicted. Based on the individual requirements of a patient, the inner skirt element may have to be adapted to the stent-structure of a prosthetic heart valve that is intended to be implant ed. In order to do so, the inner skirt element may have to be varied in its configuration as shown by the said dashed lines that may include arrowheads. The inner skirt 32 has two wings with a wing length 321, an inner skirt height 322, an inner skirt width 323 (regarding the half-cell inflow level) and an inner skirt free length of the margin 324. The inner skirt 32 has a skirt belly nadir level having a width 325. The inner skirt 32 has a length of the inflow margin 326. The scalloped inflow margin can follow the stent cells but can have different radii.

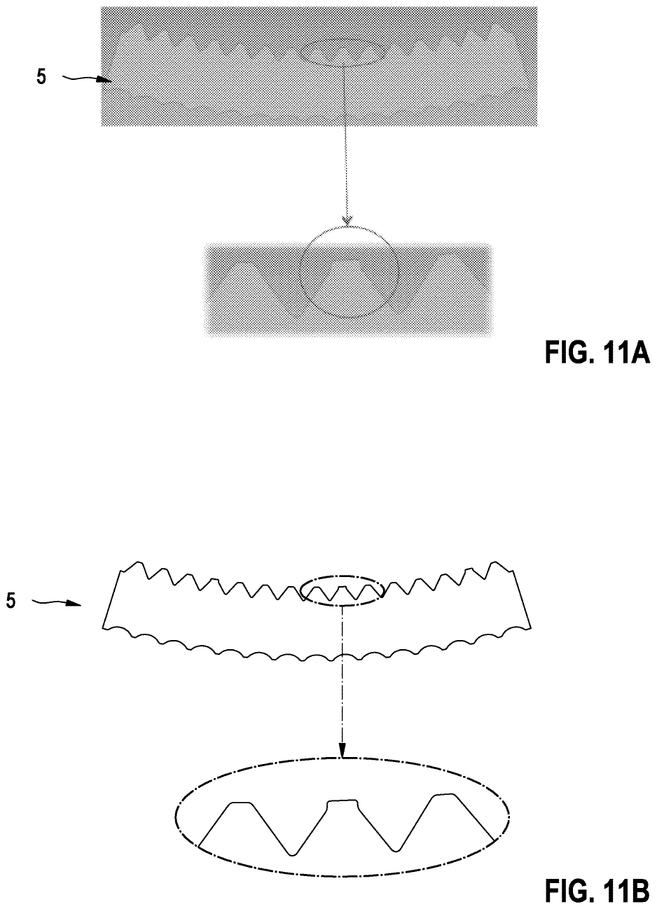

FIGS. 11A and 11B depict the general configuration of an outer skirt element 5 in accordance with the sixth aspect of the present invention and its contours as well as a detailed cutout of the distal upper portion of the said outer skirt element (Hawaiian skirt configuration). The lower proximal less scalloped portion and the upper distal more scalloped portion of the outer skirt element are both configured such that any bag formation during systole and diastole of a respective prosthetic heart valve implementing the said outer skirt element is avoided. In addition, at the belly node the respective scalloped upper distal portions of the outer skirt has been configured such that the sewing pattern of these parts is advantageous.

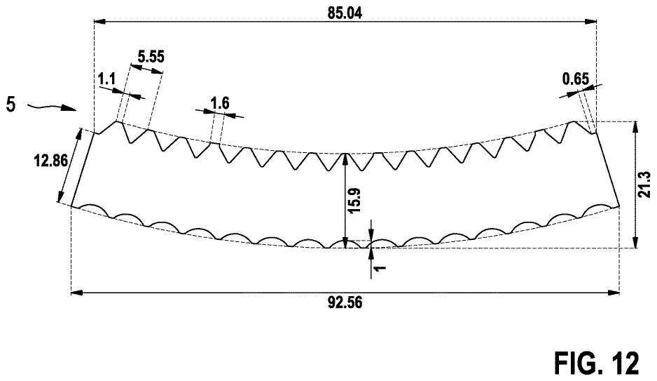

FIG. 12 depicts a schematic configuration of an outer skirt element in accordance with the sixth aspect of the present invention, its contours and exemplary dimensions thereof (Hawaiian skirt configuration). As can be derived from FIG. 11, the lower proximal less scalloped portion and the upper distal more scalloped portion of the outer skirt element are both configured such that any bag formation during systole and diastole of a respective prosthetic heart valve implementing the said outer skirt element is avoided. FIG. 12 shows exemplary dimensions of the outer skirt 5 in mm. The outer skirt 5 may have a height of between 12.86 mm to 15.90 mm and a width of between 85.04 mm and 92.56 mm.

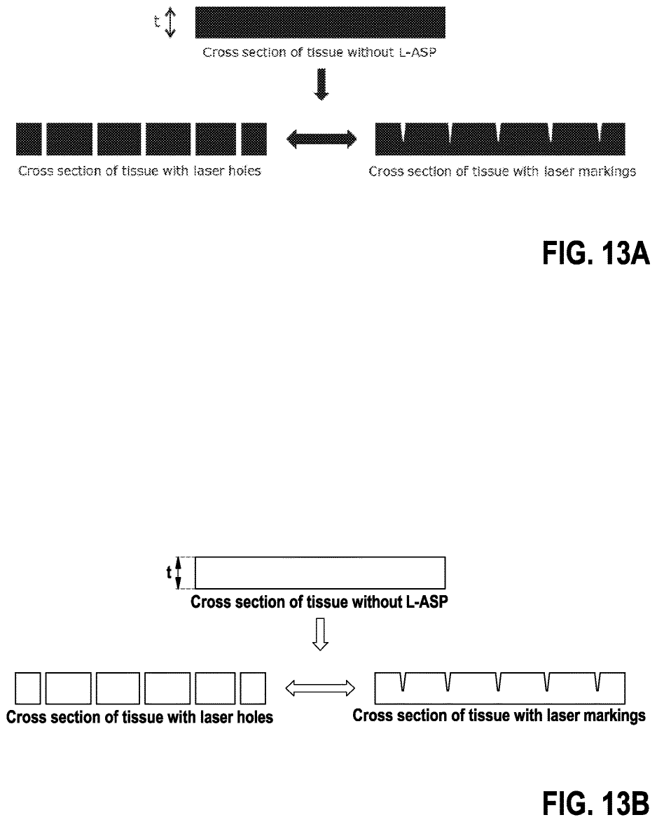

FIGS. 13A and 13B are schematic drawings regarding laser-aided sewing processes (L-ASP). The upper schematic drawing shows an exemplary tissue cross section without the use of L-ASP. The right schematic drawing shows an exemplary tissue cross section with mere laser markings obtained from L-ASP, which do not fully permeate the tissue. The left schematic drawing; however, shows an exemplary tissue cross section with complete laser through holes obtained during L-ASP. L-ASP is a generic term of a sewing assistance, which helps the operator to puncture the stitch at the right spot in accordance with the required design by indicating it on the tissue surface through respective laser scratches. Its aim is therefore to ensure design compliance, to improve the quality of the product, and therefore to increase the production yield due to the simplified process. In accordance with the present invention, L-ASP is intended to be used in order to generate laser markings or laser holes, respectively. However, using laser markings instead of full laser holes may avoid any excessive damage to the tissue such as suture hole elongation.

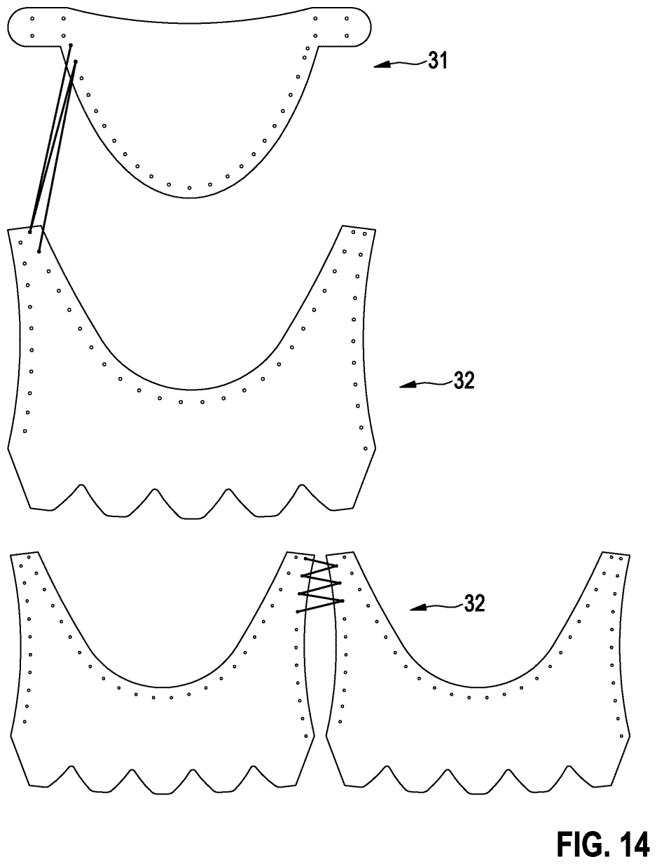

FIG. 14 is a schematic drawing of L-ASP in accordance with the present invention. L-ASP is used in the assembly procedure of the prosthetic heart valve of the invention, during the valve leaflet(s) assembly and during the skirt-to-skirt assembly. In order to minimize the number of laser markings or laser holes on the respective components an alternating sewing pattern topology can be used to assemble the subcomponents together: Hereto, the schematic drawing on the left depicts such an alternating sewing pattern topology for a skirt-leaflet assembly of the invention. On the right, the schematic drawing depicts such an alternating sewing pattern topology for a skirt-to-skirt assembly of the invention. In an alternative embodiment, fully mirrored laser markings or laser holes, meaning exactly the same markings or holes on the respective tissue components, may be used as well, but this would require more markings or holes when directly compared to the alternating sewing pattern described above. The leaflet 31 is sewn to the inner skirt, which may be made of several pieces which are sewn together as well.

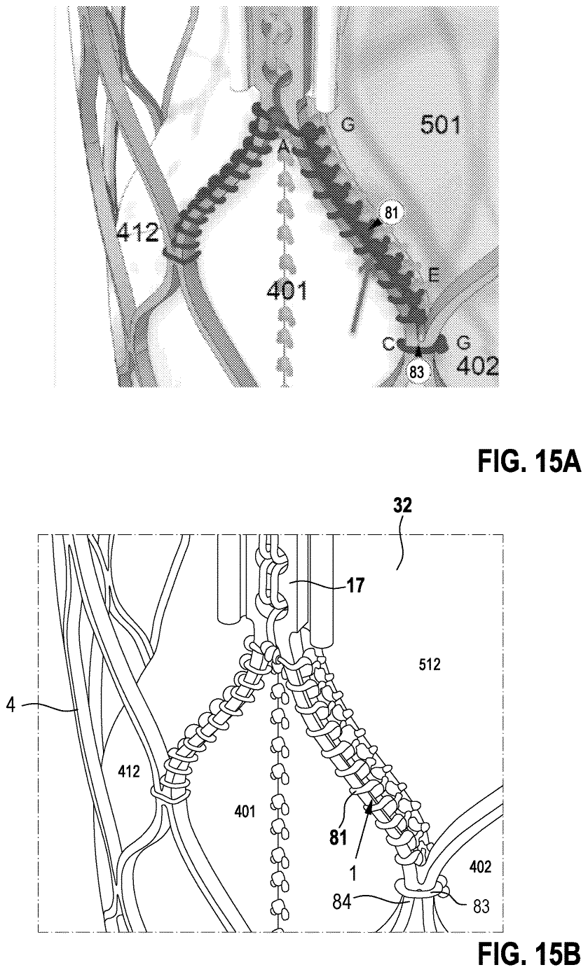

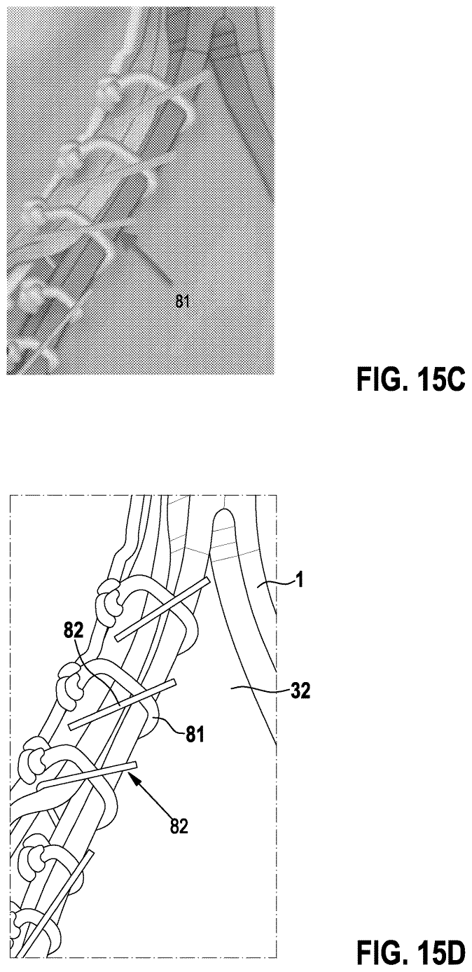

FIGS. 15A and 15C depicts an exemplary valve-stent fixation technique in accordance with the present invention. The left drawing shows that Armenian stitches are used to anchor the artificial valve component to a stent; see numeral 81 and the arrowhead therein. In the FIG. 15C it is depicted that another layer of stiches, so-called protection stitches, is applied directly over the Armenian stitches; cf. arrowhead. This is because, the higher the radial force and the reduced post-deflection of a stent is, this may lead to accelerated wearing of the suture on the stent rails (so-called suture breakage). Multiple resheathing processes of a prosthetic heart valve might also lead to suture thinning/damaging in this critical area. Accordingly, a suitable stitching pattern is required in order to prevent suture thinning/damaging on a rail of the stent and to comply with the durability requirements. Therefore, the present invention also provides for the above-mentioned overlapping two series of stitching pattern; i.e. Armenian stitches followed by additional protection stitches. In accordance with the invention, the two patterns can be of the same type or may be of two different types. The first stitching pattern anchors the valve to the rails of the stent. The second pattern goes on top of the first one to further secure the valve to the rails and protect the first set of stiches from damages during crimping and resheathing. The second set of stitches further secures the first set and limits also micro-movements of the first set of stitches. Such micro-movement may also lead to wearing. In the drawing of FIG. 15C, a first set of Armenian stitches anchors the valve to the stent rails. A second series of whipstitches overlays the said first set of Armenian stitches; cf. arrowhead. Moreover, due to this configuration of stitches, the primary stitches will not be damaged during crimping or resheathing. In addition, this sewing pattern also protects the primary stitches against any stent structure contacts and any contacts with a delivery catheter; e.g., any contacts between a delivery catheter and the prosthetic heart valve during resheathing. Further, e.g., during crimping this sewing pattern protects the stent against other stent components, e.g., two closely related struts. In view of the above, the secondary stitches reflect some sort of a buffer for the primary stitches. That is, by such a configuration of stitches only the secondary stitches may be damaged, if any.

In FIGS. 15B and 15D attachment of the valve assembly with the inner skirt 32 or the outer skirt (not shown) to the stent-structure 1 and its struts 4 is shown. FIG. 15A to 15B shows a plurality of Armenian stitches 81 forming together a first suture and a “Clove Hitch” knot 83 at a vertex of the stent-structure. As indicated in FIG. 15C to 15D, a protecting stitch structure formed by single whip stitches 82 is overlayed over the Armenian stitches 81. The protecting stitch structure has advantages as indicated above in detail. FIG. 15A shows closed cells 401,402, 412 which are formed by four struts 4 and have four vertices 84, respectively. The cell 512 is adjacent to one commissural post 17.

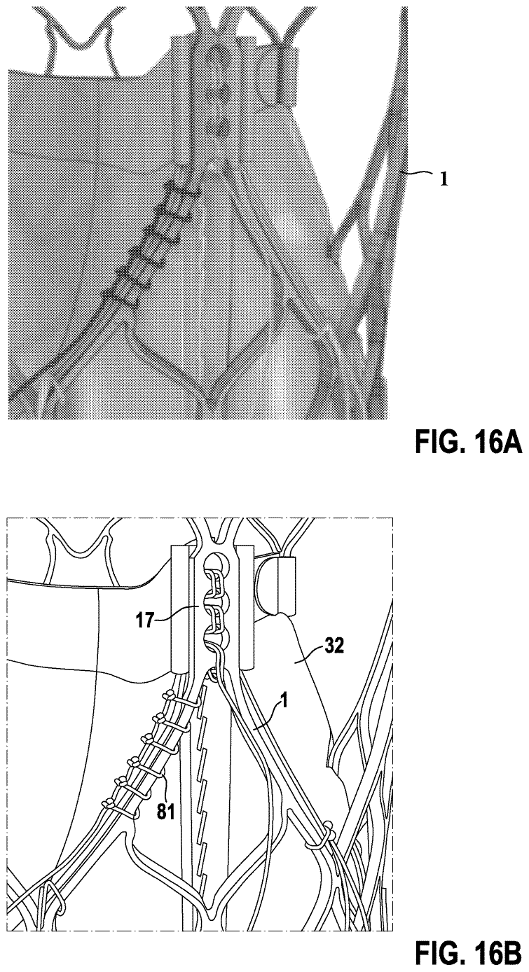

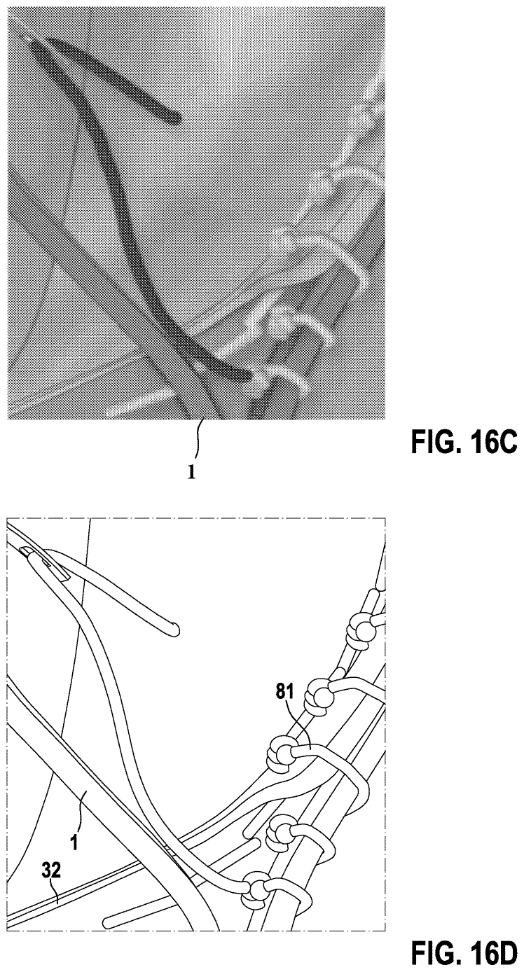

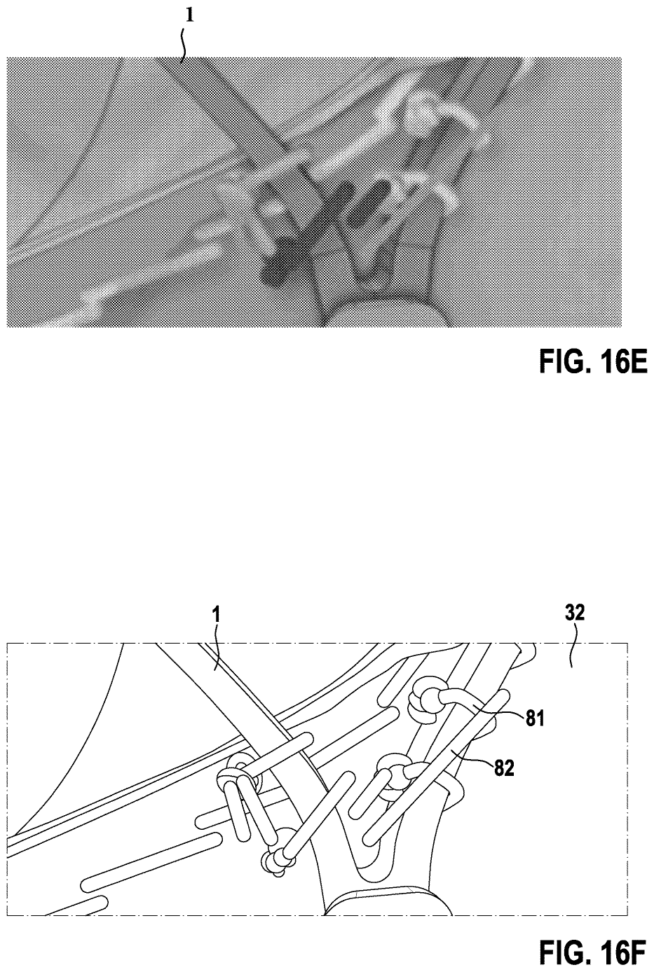

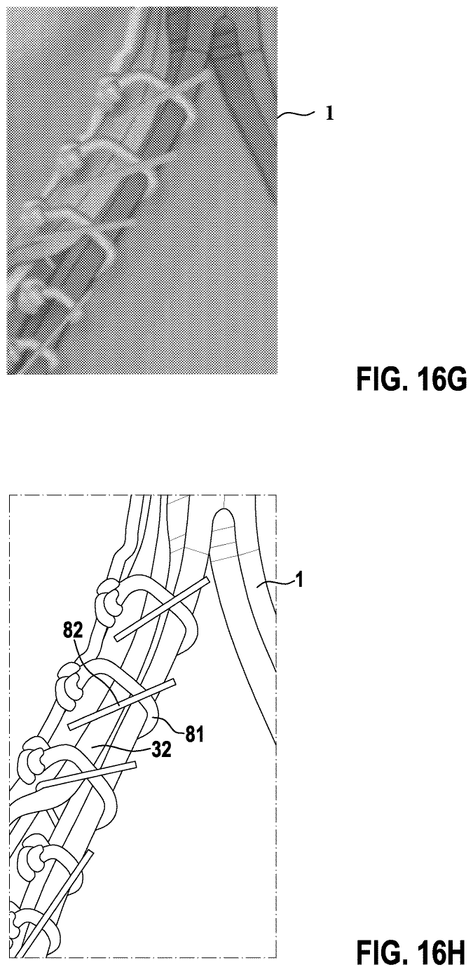

FIGS. 16A, 16C, 16E, 16G depicts an exemplary sewing procedure for a prosthetic heart valve in accordance with the present invention. The sewing pattern is characterised in that one strut in each instance is used to apply both the Armenian stitches and the protection stitches in order to reinforce the suture. The exemplary sewing procedure is as follows: Starting from the top portion of a commissural rail (commissural strut), first the Armenian stitches are used to attach the valve to the stent structure 1. Then, the Armenian stitches are continued until the bottom portion of the said rail (strut) 4. Once the Armenian stitches on the rail/strut are completed, the same thread is used «to come back up on the rails» with «whip stitches» (3). The whip stiches (i.e. the protection stiches) thereby overlay the Armenian stitches (first sewing pattern) for the complete length of the rail/strut and continues until the commissural tabs where they are afterwards secured (4).

FIGS. 16B, 16D, 16F, 16H show the suturing procedure of the first suture and the protecting stitch structure, wherein at first the Armenian stitches 81 are provided in order to secure the inner skirt 32 at a strut of the stent-structure 1 (see FIGS. 16A to 16D). Starting from the top portion of a commissural posts 17 (also denoted as commissural struts, commissural tabs or commissural rails), first the Armenian stitches 81 are used to attach the inner skirt 32 to the stent-structure 1. Then, the Armenian stitches 81 are continued until the bottom portion of the said strut (FIGS. 16A to 16D). Once the Armenian stitches 81 on the strut are completed, the same thread is used to come back up on the struts with whip stitches 82 (FIGS. 16E to 16H). The whip stiches 82 thereby overlay the Armenian stitches 81 (first sewing pattern) for the complete length of the strut and continue until the commissural posts where they are afterwards secured.

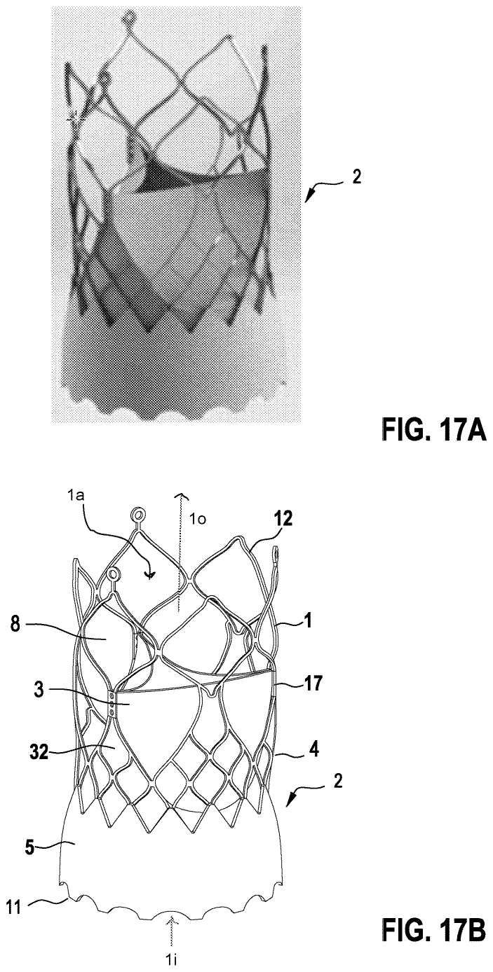

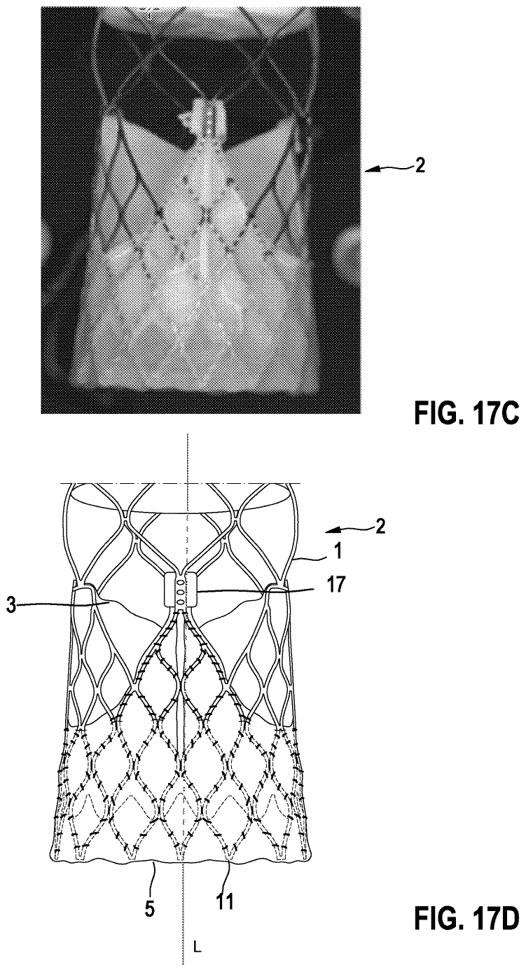

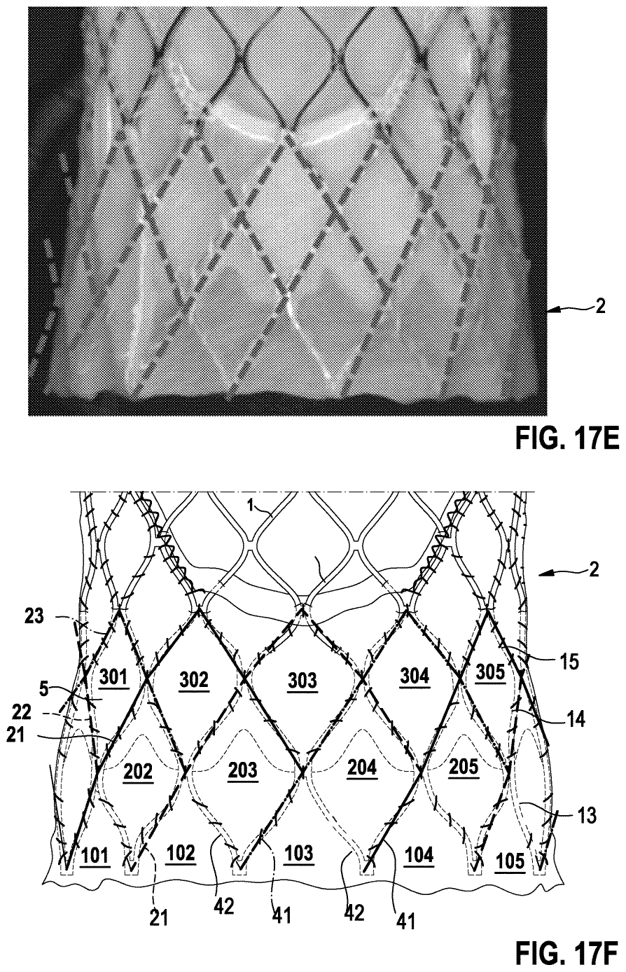

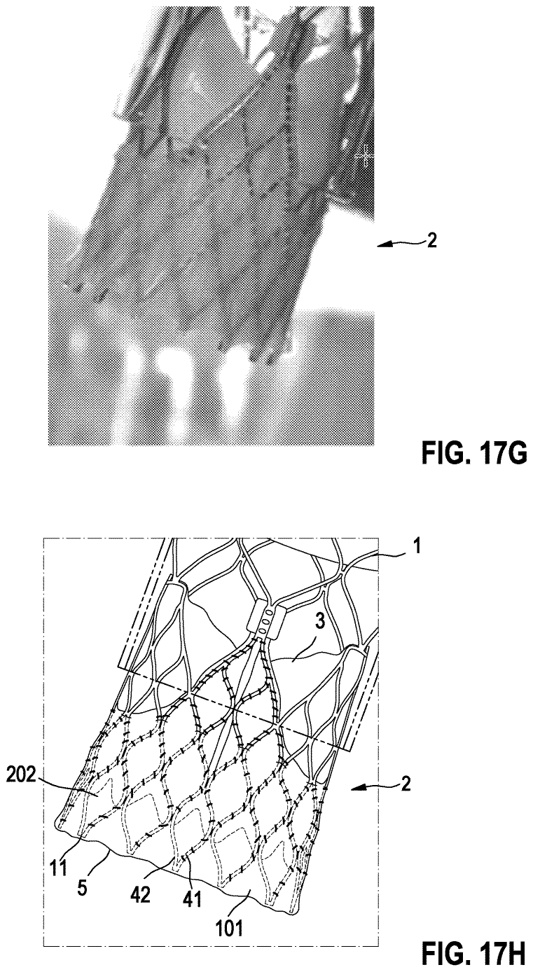

FIGS. 17A, 17C, 17E, 17G show photos of various exemplary embodiments of an outer skirt element in accordance with the sixth aspect of the present invention (Hawaiian skirt configuration). The Hawaiian skirt configuration as schematically shown in FIG. 17A) is configured in order to prevent the occurrence of any paravalvular leakage by augmenting the contact surface with the anatomical structures within the annular regions (also in the surrounding areas, subannular (LVOT) and suprannular regions (native leaflet)). To this end, the above-described sewing pattern is chosen such that the resulting unconstrained outer skirt surface is folding when the prosthesis is crimped down to the IFU implantation diameters and thereby augmenting the contact with the anatomical structures. In the Hawaiian skirt configuration, the circumferential length of the skirt equals the stent at the belly level and at the inflow level. The height of the skirt equals the height of the stent between the inflow and the belly nadir. By selectively limiting the stitching in the first meander row of the stent (as shown in FIG. 17E) by the dashed lines), the resulting unconstrained outer skirt surface is folding when the prosthesis is crimped down to the IFU implantation diameter range (see FIG. 17G)), assuming a shape that looks similar to an Hawaiian skirt. The Hawaiian skirt configuration is configured such that no blood is intended to be trapped in this configuration. The Hawaiian skirt may be made from pericardium such as porcine or bovine pericardium or from a fabric such as polyethylene or polyurethane. Also in accordance with FIG. 10A, the top (belly nadir) of the Hawaiian skirt configuration (i.e. the upper distal portion) is saw-shaped to follow the stent. The peaks thereof have all the same height. It is sewn together with the inner skirt element in accordance with the invention, but in some struts/rails the outer skirt elements in the Hawaiian skirt configuration is left unconstrained (see FIG. 17E), the dashed lines highlighting the respective sewing pattern). In one embodiment, the proximal bottom portion at the inflow can be selected from being (i) saw-shaped or (ii) scalloped.

The heart valve prosthesis 2 in FIG. 17B shows a drawing of the photo of FIG. 17A. The heart valve prosthesis includes a stent-structure 1 and a valve assembly 3. The stent-structure has a compressed state (not shown) and a functional state (shown). The stent-structure 1 has a first stent-structure end 11 and a second stent-structure end 12, a longitudinal axis (not shown) and a plurality of struts 4 forming a plurality of cells.

The stent-structure (1) surrounds an inner volume (8) wherein the valve assembly 3 is situated. The valve assembly 3 is affixed to commissural posts 17 of the stent-structure (1).

The heart valve prosthesis includes an outer skirt 5 partially covering the outer side of the stent-structure 1 and an inner skirt 32 covering at least partially an inner side of the stent-structure 1.

The stent-structure has a (proximal) inflow section and an outflow section and at least one intermediate section (also denoted as transition zone) arranged between said inflow and outflow section. The inflow section of the stent-structure, the inner skirt 32 (located at the intermediate section of the stent-structure) the valve assembly 3 (located at the intermediate section of the stent-structure), and optionally the outer skirt 5 (located inflow section of the stent-structure), together form an implant component. The medical implant component defines a flow passage for guided and/or controlled fluid flow (e.g. blood flow). The flow passage has an inflow end (located at the inflow section of the stent-structure) and an outflow end (located at the intermediate section of the stent-structure). The first stent-structure end 11 is situated at the inflow end of the flow passage. The second stent-structure end 12 is situated spaced apart from the outflow end of the flow passage (when looking along the longitudinal axis not shown here but see FIG. 27).

FIG. 17D shows a drawing of the photo of the medical implant 2 of FIG. 17C. The medical implant 2 includes a stent-structure 1, a valve assembly 3 and an outer skirt 5 partially covering the outer side of the stent-structure 1 and together forming an implant component.

The stent-structure 1 has a first stent-structure end 11 and a second stent-structure end (not shown) and a plurality of struts 4. The stent-structure 1 surrounds an inner volume 8 wherein the valve assembly 3 is situated. The valve assembly 3 is affixed to commissural posts 17 of the stent-structure 1.

The medical implant component defines a flow passage for guided and/or controlled fluid flow (e.g. blood flow). The flow passage has an inflow end (first stent-structure end 11) and an outflow end (being spaced apart from the second stent-structure end 12 in longitudinal direction (which is along the longitudinal axis).

The stent-structure may have a compressed state and may be expandable in a functional state.

FIG. 17F shows a drawing of the photo of the medical implant 2 of FIG. 17E (which is a close-up of FIGS. 17C and 17D).

The medical implant has a stent-structure 1 with a plurality of struts 4 forming one or more zig-zag structures, meander structures or wave line structures, wherein each zig-zag structure, meander structure or wave line structure extends around the full circumference of the stent-structure 1. One zig-zag structure, meander structure or wave line structure at the stent-structure end 11 forms a half-cell 101, 102, 103, 104, 105. A half-cell is surrounded by a first strut 41 and a second strut 42. Two zig-zag structures, meander structures or wave line structures are connected so as to form closed cells 202, 203, 204, 205.

It can be seen that the outer skirt is affixed (e.g. sutured) to the stent-structure to each second strut 42 of the half-cells along the full circumference of the stent-structure, thereby forming pleats (in a fold creating section). These pleats enable better sealing of the medical implant. The fixing is done by three sutures 21, 22, 23 attached to the struts of the stent-structure. Accordingly, along the zig-zag structure 13 of the stent-structure 1 a fold creating section of the outer skirt 5 is formed. Further, along the zig-zag structures 14, 15, the outer skirt 5 is affixed to each strut thereby creating a sealing section.

The heart valve prosthesis 2 in FIG. 17H shows a drawing of the photo of FIG. 17G. The heart valve prosthesis 2 has a stent-structure 1 having a plurality of closed cells 202 and a plurality of half-cells 101, both being formed by a plurality of struts. The half-cells are situated at the first stent-structure end 11 and the second stent-structure end (not visisble), respectively. A half-cell is formed by one first strut 41 and one second strut 42. At least one of the closed cells is formed by the first strut 41 and the second strut 42 and two further struts. The heart valve prothesis 2 has an outer skirt 5 which is affixed to each second strut 42 of the half-cell located at the first-structure end 11. The outer skirt 5 is affixed to each closed cell being connected to the half-cells located at the first-structure end 11. The heart valve prosthesis 2 has a valve assembly 3 being attached on in the inner side of the stent structure 1.

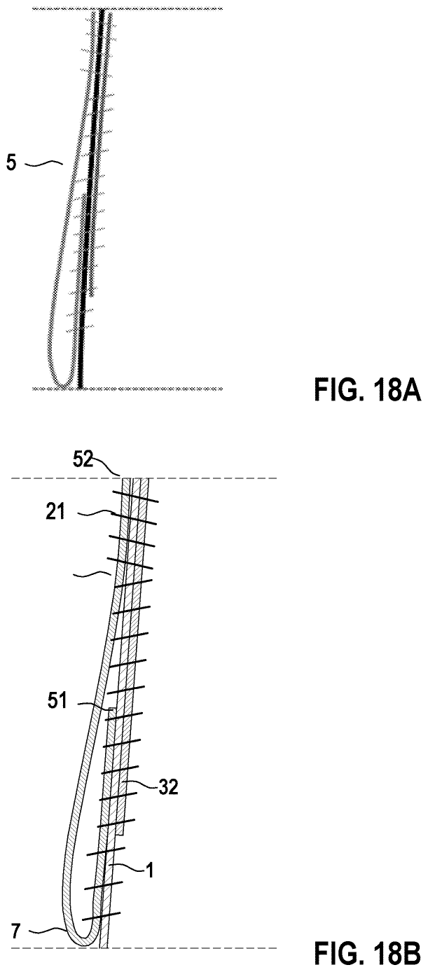

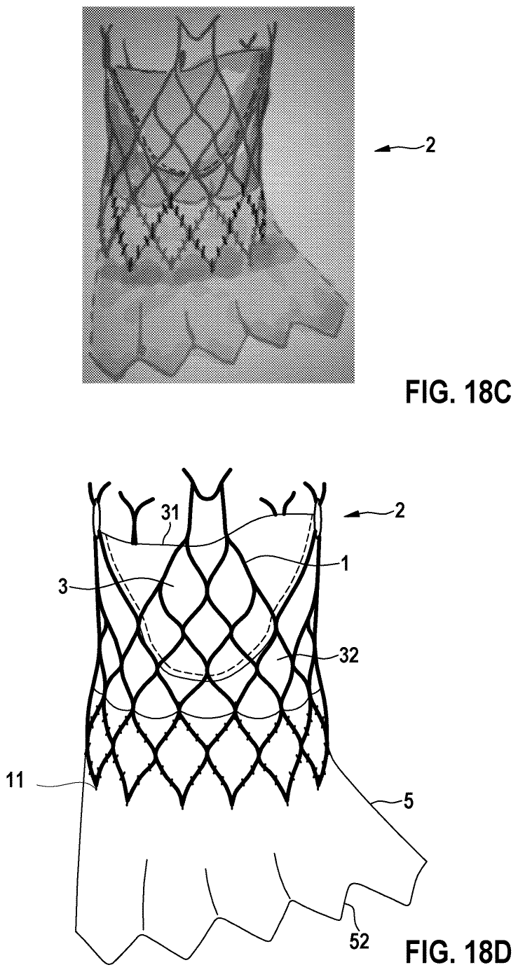

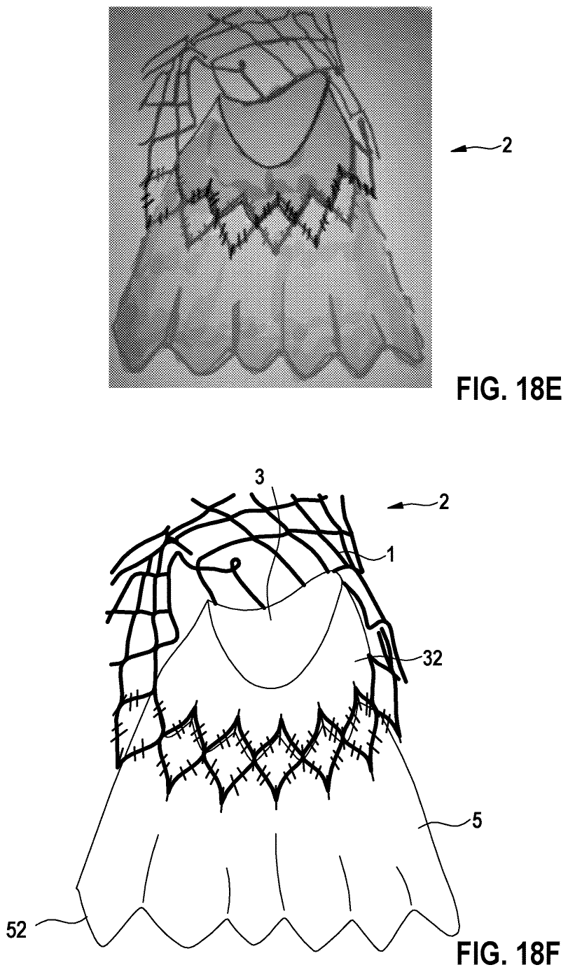

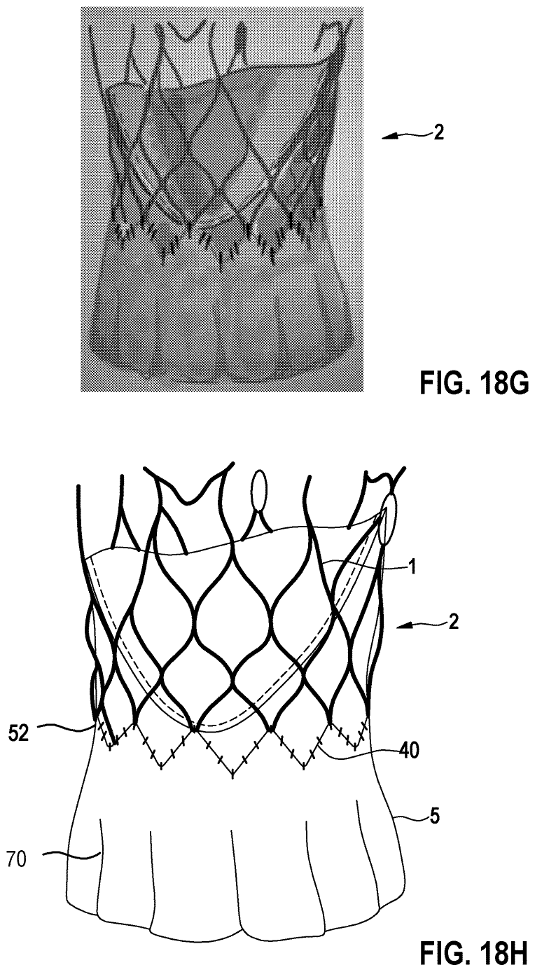

FIGS. 18A), 18C), 18E and 18G) show various exemplary embodiments of an outer skirt 5 element in accordance with the seventh aspect of the present invention (Primula configuration). The Primula configuration as schematically shown in FIGS. 18A), 18C), 18E and 18G is configured in order to prevent the occurrence of any paravalvular leakage by augmenting the contact surface with the anatomical structures within the annular regions (also in the surrounding areas, subannular (LVOT) and suprannular regions (native leaflet)). To this end, the outer skirt element in the Primula configuration adopts a gasket-like structure. That is, in the Primula skirt configuration the circumferential length of the skirt equals the stent at the belly level and at the inflow level. The height of the outer skirt is longer than the height of the stent between the belly nadir and the inflow of the stent because the outer skirt of the Primula configuration is supposed to be folded, along the longitudinal axis of the stent, during the assembly of the heart valve prosthesis. This will create a sort of gasket-like structure (formed as a consequence of the superimposition of at least 2 layers of pericardium). Moreover, the outer skirt in the Primula configuration can be forced to have no/negligible axial slack by being constrained through stitching specific spots of the outer layer on the stent to drive the fold only circumferentially. The outer skirt element of the Primula configuration may be made from pericardium such as porcine or bovine pericardium or from a fabric such as polyethylene or polyurethane. FIGS. 18C to 18H show a medical implant 2 with a valve assembly 3 having leaflets 31 and one outer skirt 5 in a Primula configuration. As can be seen in FIG. 18A the outer skirt has a first outer skirt edge 51 and a second outer skirt edge 52 and is folded between the first outer skirt edge 51 and the second outer skirt edge 52 and thereby forming a hook like or loop like structure 7 double layered outer skirt. The hook like or loop like structure 7 is situated at the first stent-structure end 11 and/or at the inflow end of the flow passage. The first outer skirt edge is affixed to the inner skirt 32 and the stent-structure 1 and the second outer skirt edge 52 is affixed to the inner skirt 32 and the stent-structure 1. FIGS. 18D, 18F, 18H show the sequence of manufacturing steps of a Primula configuration. In FIG. 18D the outer skirt 5 is placed on the outer surface of the stent-structure 1 and is assembled together with (the proximal portion of) the inner skirt 32. The outer skirt 5 is the fully affixed to the stent-structure 1 up to first stent-structure end 11. FIG. 18F shows a view from the inside of the prosthesis. FIG. 18H shows that outer skirt 5 is folded on itself and finally constrained together to the inner skirt 32 at the half cell (right below the belly nadir) along the circumference of the stent-structure 1. Additionally, to the hook like or loop like structure 7 the outer skirt 5 may have pleats 70.

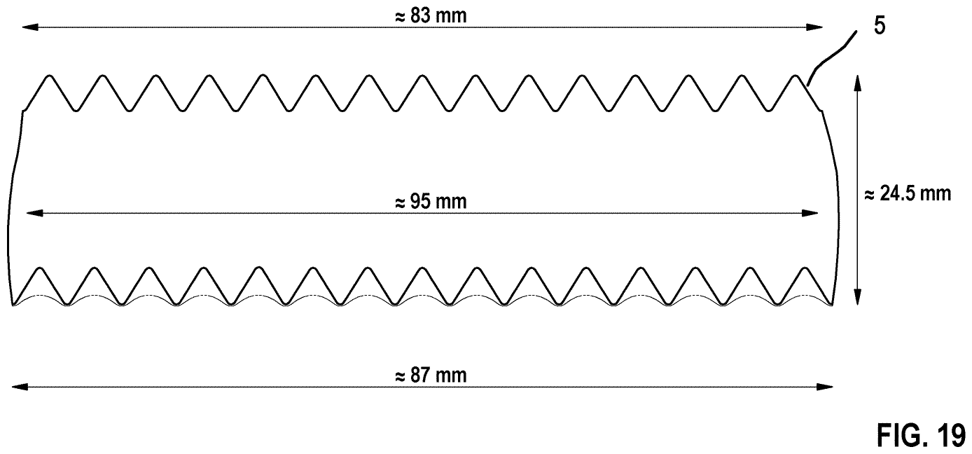

FIG. 19 depicts exemplary contours of the outer skirt element of the invention in the Primula configuration with respective dimensions. As can be derived from FIG. 19, the upper distal portion and the lower proximal portion of the outer skirt element can both be saw-shaped in one embodiment. In another embodiment, the lower proximal portion of the outer skirt element of the Primula configuration can be scalloped as depicted by the dashed lines. FIG. 19 shows exemplary dimensions of the outer skirt 5 in mm. The outer skirt 5 may have a height of between 24.5 mm and a width of between 83 mm and 95 mm.

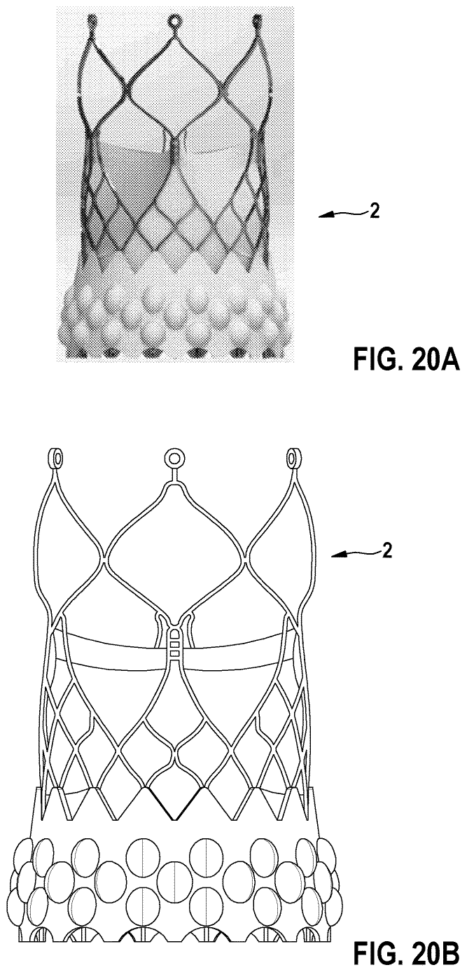

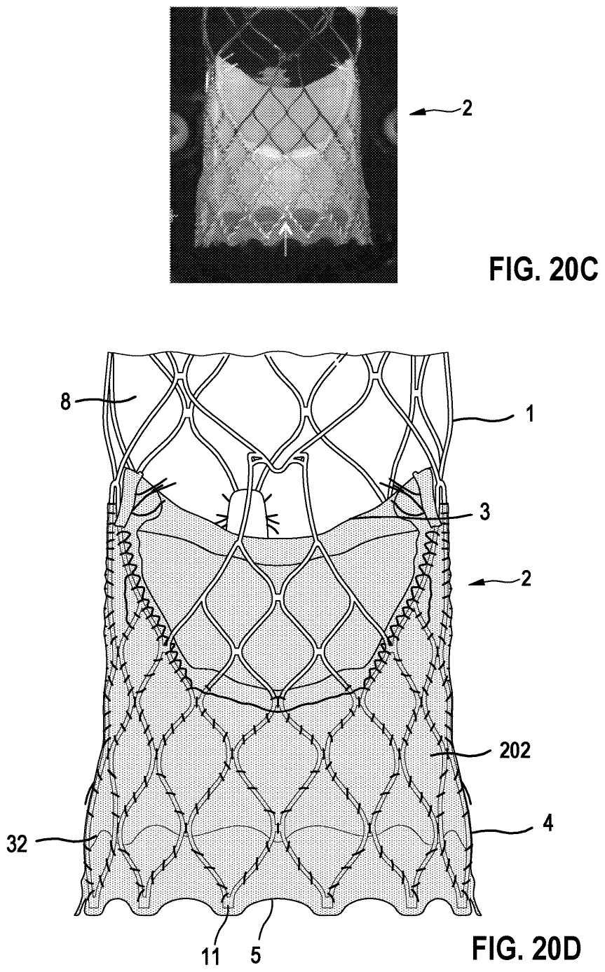

FIG. 20A) to 20D) show two exemplary embodiments of an outer skirt element in accordance with the eight aspect of the present invention (3D shape configuration). The 3D shape configuration is configured in order to prevent the occurrence of any paravalvular leakage by increasing the contact surface with the anatomical structures within the annular regions (also in the surrounding areas, subannular (LVOT) and suprannular regions (native leaflet)); especially in the presence of any calcific nodules and in order to increase the hydraulic resistance to the backflow. Therefore, the 3D shape configuration includes an outer shell structure that is shaped with one or more structures elevating/bulging from the shell plane circumferentially such as one or more bubble-like structures as shown in FIGS. 20A) and 20C). In one embodiment of the invention, these elevating/bulging structures can be of different size and shape and are thus not limited to bubble-like structures. In one embodiment, the said elevated/bulging structures are already present when the prosthetic heart valve of the invention is in the fully expanded state; cf. FIG. 20A). In the 3D shape configuration, the circumferential length of the outer skirt element equals the stent at the belly level and at the inflow level. The height of the outer skirt element equals the height of the stent between the inflow and the belly nadir. Also, with the 3D shape configuration it is intended that no blood can be trapped, e.g., that may form any bags. When the prosthetic heart valve of the invention is crimped down to the IFU implantation diameters, the elevating/bulging structures will be slightly readapting in shape due to the stent geometrical change. The upper distal portion of the 3D shape outer skirt element (belly nadir) is saw-shaped in order to follow the stent's cell contours (cf. FIG. 20A)). The respective peaks are all the same height and are also sewn together with an inner skirt element of the present invention. The lower proximal portion of the 3D shape outer skirt element (at inflow of the stent) can be selected from being (i) saw-shaped or (ii) scalloped. The outer skirt element of the 3D shape configuration may be made from pericardium such as porcine or bovine pericardium or from a fabric such as polyethylene or polyurethane.

FIG. 20D shows of a medical implant 2 in a functional state. The the medical implant 2, preferably a transcatheter deliverable endoprosthesis, includes a stent-structure 1, an inner skirt 32 and an outer skirt 5 and a valve assembly 3. The stent-structure has a first stent-structure end 11 and a second stent-structure end (not shown) being opposite to the first stent-structure end 11, and wherein the stent-structure 1 surrounds an inner volume 8 and wherein the stent-structure 1 has a plurality of struts 4 forming cells 101. The outer skirt 5 partially covers an outer side of the stent-structure 1 and fully covers the first stent-structure end 11. The inner skirt 32 partially covers an inner side of the stent-structure 1. The valve assembly 3 is arranged within the inner volume 8 of the stent-structure.

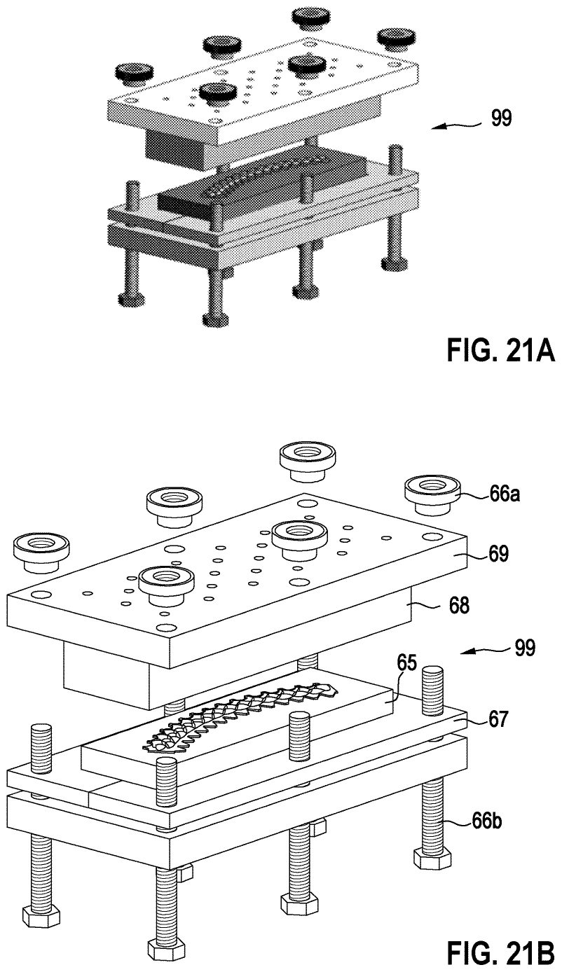

FIGS. 21A and 21B depicts an apparatus 99 for the manufacture of an outer skirt element of the present invention of the 3D shape configuration; cf. FIG. 20. If said outer skirt element is made from pericardium such as porcine pericardium, it is obtained from porcine pericardium by a process in which the pericardium is compressed within two shells: i) a positive POM-shaping-mold and a negative polyurethane sponge of the final shape while having a specific treatment with glutaraldehyde (GA), namely over 4 to 14 days with a glutaraldehyde change every 2 days in order to chemically cross-link the tissue in the desired 3D shape; e.g., with different elevated/bulging structures. During this process, the temperature, applied pressure and other physical parameters are controlled.

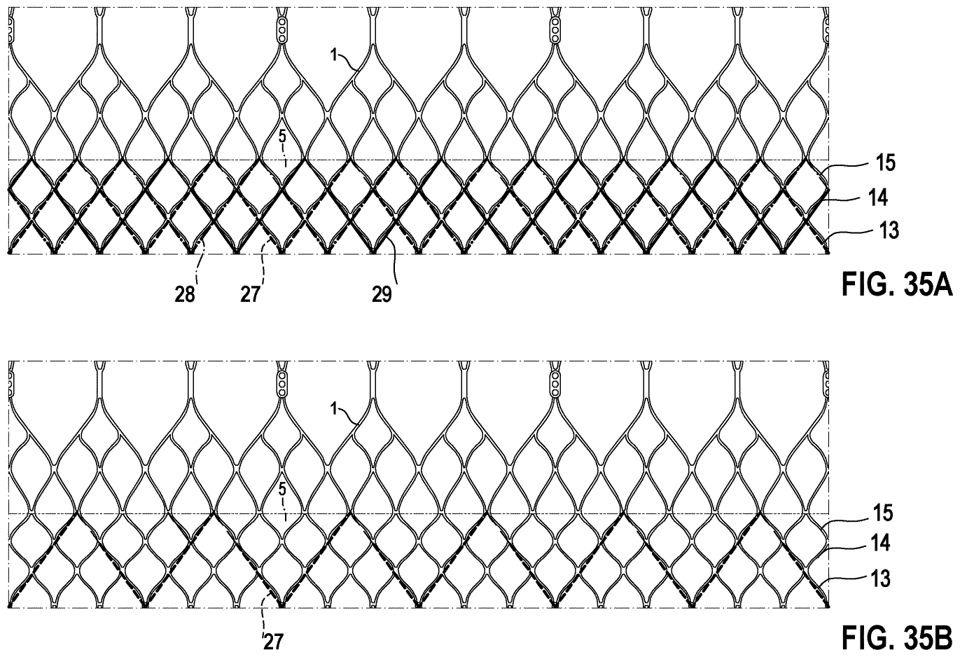

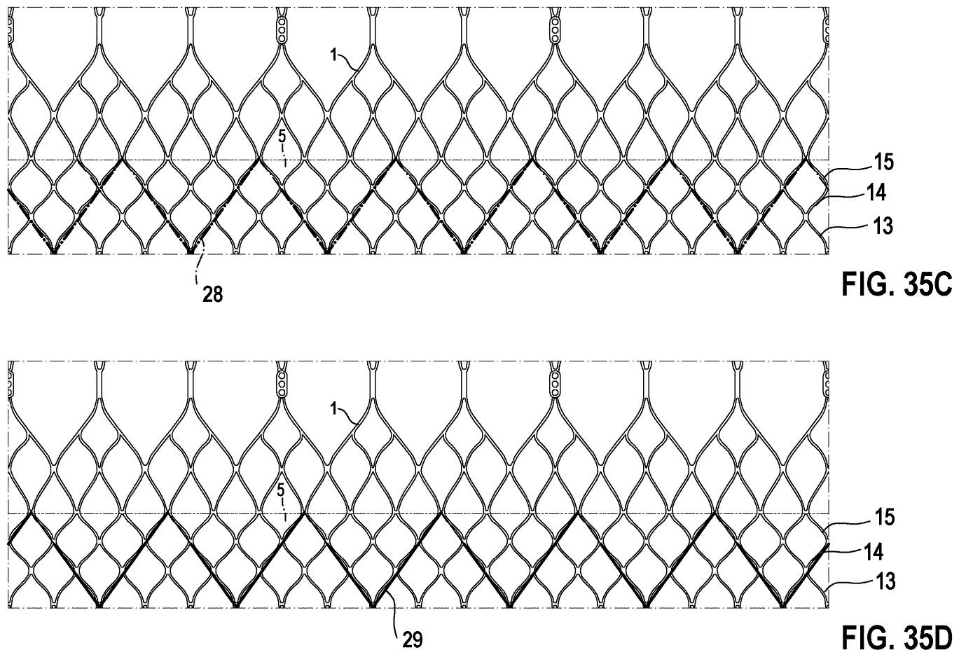

FIG. 21B shows an apparatus for the manufacture of an 3D shaped outer skirt element (of FIG. 35A) having a top-plate 69 (with holes enabling a solvent to pass through), a shaping mold holder 67 having a 3D shaping mold 65. The shaping mold holder and the 3D shaping mold can be one or more pieces. The top-plate 69 can be affixed on top of the 3D-shaping mold 65 and/or the shaping mold holder 67 by fixation structures 66a, 66b. Between the top plate 69 and the 3D-shaping mold 65 and/or the shaping-mold-holder a sponge 68 can be arranged. The sponge 68 may have a compression hardness of 60 kPa.

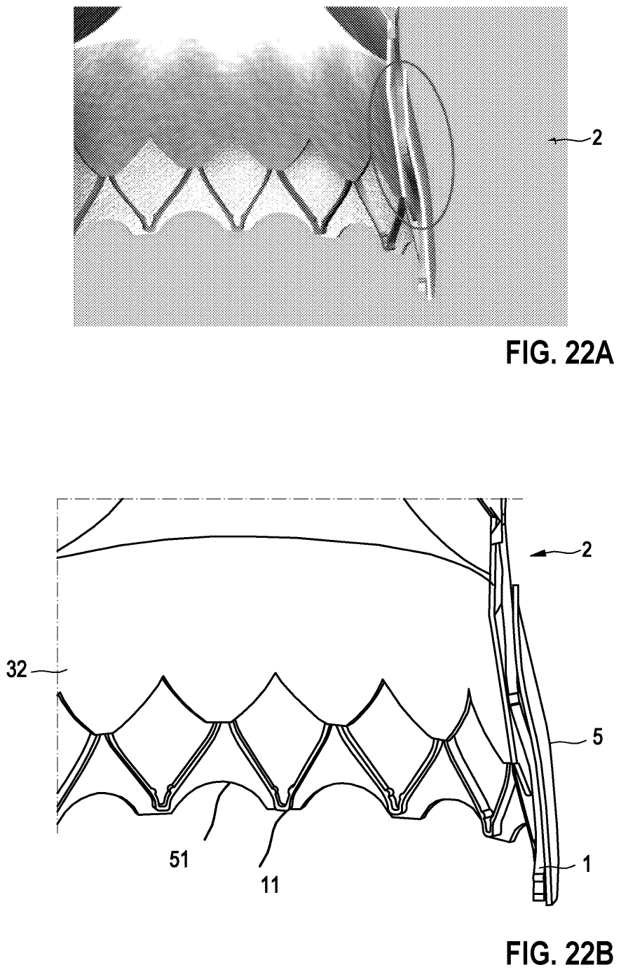

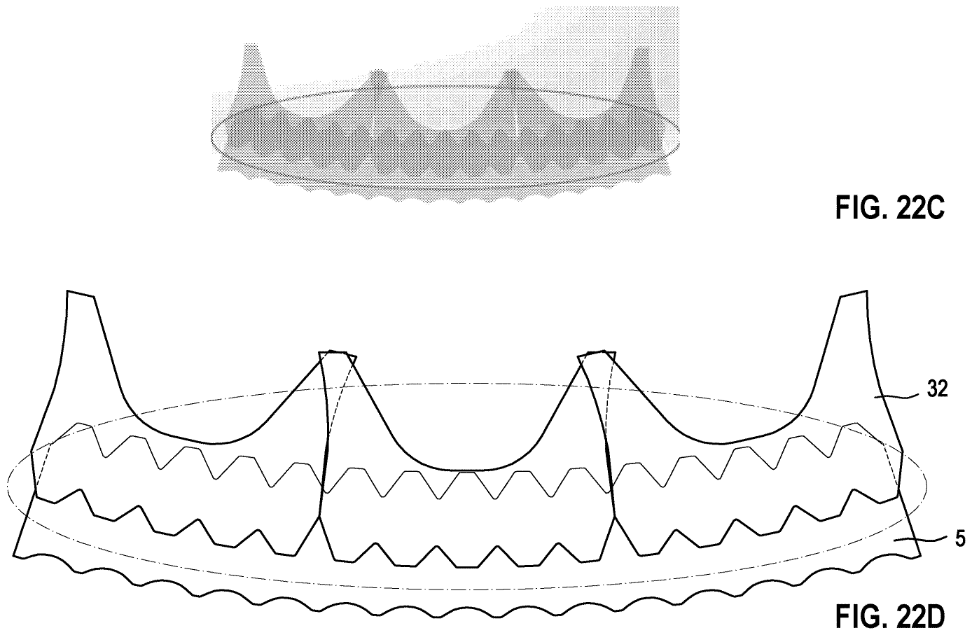

FIGS. 22A and 22C are two schematic drawings, which reflect the general overlap of the inner skirt elements with the outer skirt elements in accordance with the present invention. In the FIGS. 22C and 22C one can derive that in this embodiment the inner skirt has a saw-shaped upper distal portion and a saw-shaped lower proximal portion in order to be densely and completely sutured to the contours of the stent structure's cells and thus to avoid any bag formation to not trap any blood.

In the medical implant 2 of FIG. 22B the outer skirt 5 is partially affixed to the inner skirt 32 and/or the stent-structure 1. However, the first outer skirt edge 51 is affixed to the first stent-structure end 11 only. 8. The inner skirt 32 does not cover the first stent-structure end 11 and does not extend to the first stent-structure end 11.

FIG. 22D show the overlap of an outer skirt 5 with an inner skirt 32, made of three pieces being attached to each other

FIGS. 23A and 23B: Schematic representation of the outer contours of a stent structure in accordance with the present invention. Z1 denotes a conical-convex inflow region/section (annulus zone) defined by a first diameter D1 and a second diameter D2 (“Belly Nadir” diameter), D1 being greater than D2 and the outer surface of this Z1 region being characterized in that it is curved outwards in the longitudinal direction (convex or double curved, respectively). The region Z1 is followed by a single conical valve zone Z2 (valve zone) with a cone opposite to Z1, Z2 being defined by a first diameter D2, which can be the smallest diameter of the entire vascular implant, and a second diameter D3 (so-called attachment diameter), further characterized in that the first diameter D2 is smaller than the second diameter D3. Zone Z2 is followed by a cylindrical and thus rectilinear outflow zone Z3 (outflow zone), characterised in that the diameter D3 remains the same throughout zone Z3. This leads to a so-called “straight outflow” of the vascular implant. Finally, a connector zone Z4 follows. In one design, the connector zone can consist of 3 connectors. In a further design, the connectors, optionally three, can be single-stranded and curved inwards and also have atraumatic tip elements (not shown). For example, the first diameter D1 may be 29.5 mm, the second diameter D2 may be 25.5 mm and the third diameter D3 may be 26.5 mm.

FIG. 24: Representation of a stent structure in accordance with the present invention and based on the characteristics of FIG. 23. Z1 denotes a conical-convex zone defined by a first diameter D1 and a second diameter D2 (“Belly Nadir” diameter), where D1 is larger than D2 and the outer surface of this Z1 zone is characterized in that it is curved outwards in the longitudinal direction (convex or double curved, respectively). The zone Z1 is followed by a single conical valve zone Z2 (valve zone) with a conical shape opposite to Z1, where Z2 is defined by a first diameter D2, which can be the smallest diameter of the entire vascular implant, and a second diameter D3 (so-called attachment diameter), further characterized in that the first diameter D2 is smaller than the second diameter D3. Zone Z2 is followed by a cylindrical and thus rectilinear outflow zone Z3 (outflow zone), characterised in that the diameter D3 remains the same throughout zone Z3. This leads to a so-called “straight outflow” of the prosthetic heart valve. The upper boundary of the stent structure is a connector zone Z4, which can consist of 3 connectors in the design shown. In one design, the connectors, optionally three, can be single stranded, bent inwards and have atraumatic tip elements (not shown).

FIG. 25: Representation of a stent structure in accordance with the present invention and based on the characteristics of FIG. 23. The shown design is characterized by a stent structure having a certain number of cells in the circumferential direction in the area of inflow a), said number of cells being divisible by 3 to ensure a ⅓ symmetry, further characterized in that a further number of cells in the circumferential direction is present in the outflow area b), which is also divisible by 3, but which is lower than the number of cells in said area of inflow a). Both areas a) and b) are connected by a so-called transition area c) (transition zone), which ensures the connection between inflow area a) and outflow area b) and in this configuration includes the largest cells of the stent structure 3) for free access to the coronary arteries. In a preferred configuration according to FIG. 25, the inflow zone includes 12, 15 or 18 cells (1) and the outflow zone 3, 6 or 9 cells (2).

In the following the invention is explained with respect to various embodiments of a heart valve prosthesis. However, the invention may similarly be used in other medical implants, for example other (transcatheter deliverable) endoprosthesis or stent-grafts as well.

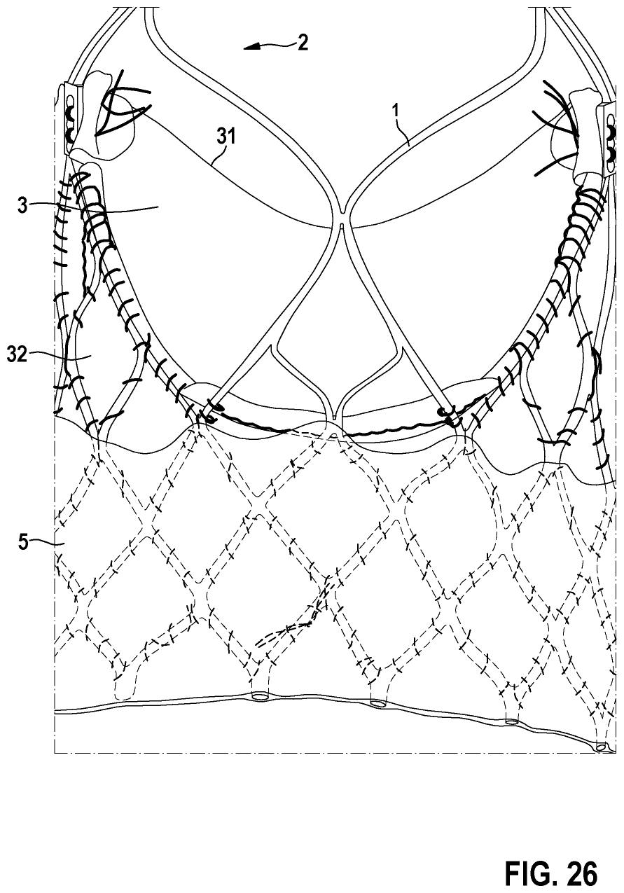

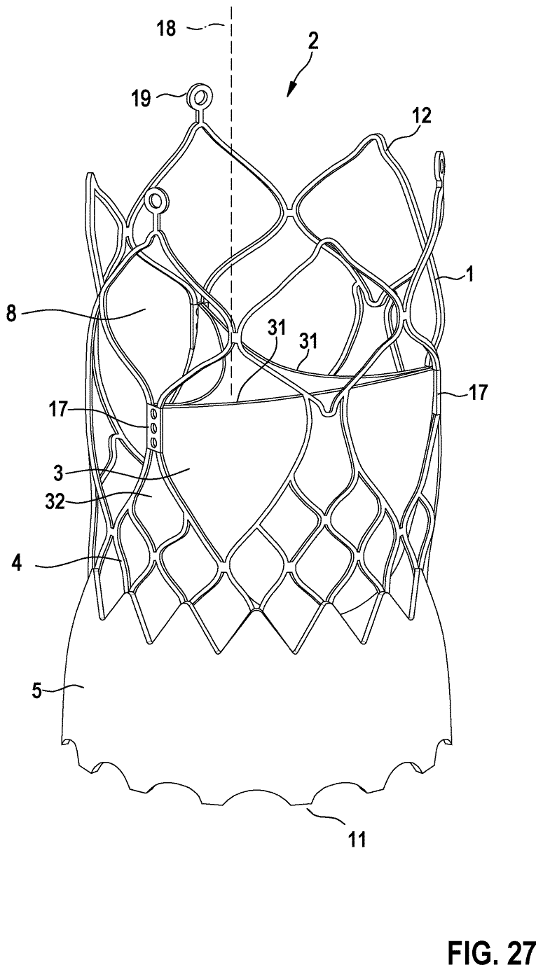

A first embodiment of a heart valve prosthesis is explained with regard to FIG. 26 to 27 and 29 to 32. The heart valve prosthesis includes a tubular stent-structure 1 and a valve assembly 3, both forming an implant component. The stent-structure has a compressed state (not shown) and a functional state (shown in FIGS. 26 and 27). Further, the stent-structure 1 has a first stent-structure end 11 and a second stent-structure end 12 (see FIG. 27), a longitudinal axis 18 (see FIG. 27) and a plurality of zig-zag structures 13, 14, 15 forming a cell pattern with a plurality of cells (see FIG. 29 to FIG. 32) and a contour of the medical implant component. Each closed cell (for example the cells 202, 203, 204, 205) near the first stent-structure end 11 of the stent-structure is formed by two adjacent zig-zag structures (for example zig-zag structures 13, 14) fixed to each other at the respective vertices. Accordingly, each closed cell is formed by four struts. The cells have different size along the prosthesis. At the second stent-structure end holding structure (e.g. eyelets) for coupling with a catheter can be situated.

The valve assembly 3 is located within the stent-structure 1 and has three leaflets 31 and an inner skirt 32, wherein the leaflets 31 and the inner skirt 32 are affixed to each other by suturing in a known way. Further, the valve assembly 3 is affixed to the stent-structure 1 by suturing, for example at commissural posts 17.

The prosthesis defines a flow passage through the valve assembly 3 from the first stent-structure end 11 (forming the inflow section) to the end of the valve assembly 3 opposite the first stent-structure end 11 (forming the outflow section). The fluid flow (e.g. the blood flow) through the flow passage is controlled by the leaflets 31 which may adopt a closed state and an open state.

The outer diameter of the stent-structure 1 is greater at the first stent-structure end 11 than at an intermediate section. Further, the outer diameter of the stent-structure 1 at the intermediate section is less than the outer diameter of the stent-structure at the second stent-structure end 12. The level of the stent-structure along its longitudinal axis 18 with the smallest diameter is called belly nadir.

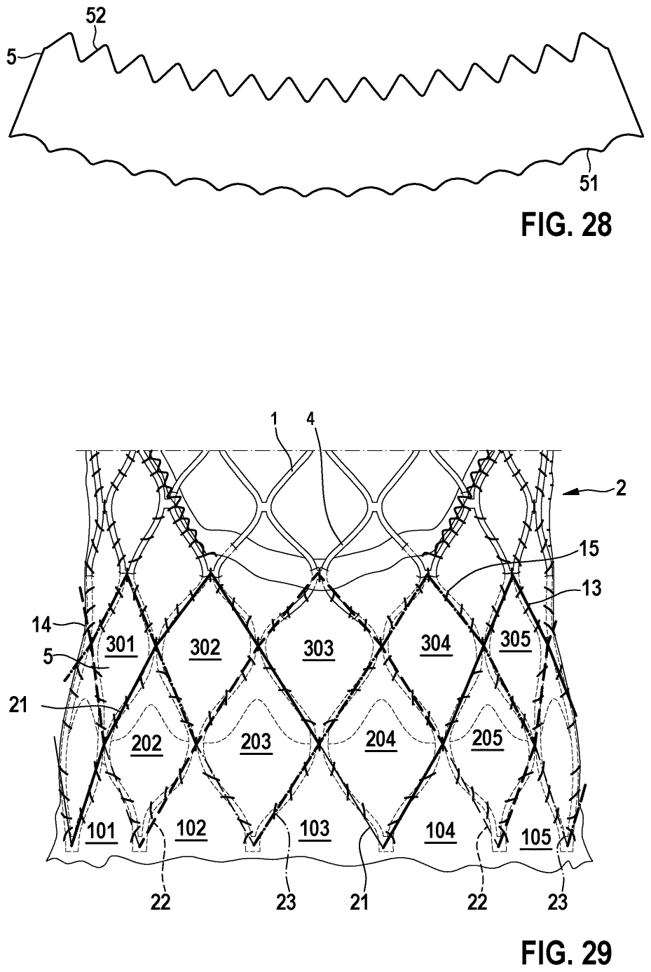

Furthermore, the prosthesis includes an outer skirt 5 shown in FIG. 28 before attachment at the stent-structure. The outer skirt 5 is a rectangular (biological) tissue which is slightly curved. The first edge 51 of the outer skirt has a scalloped shape, whereas the second edge 52 of the outer skirt 5 is saw-shaped. The peaks of the shapes at the first edge 51 and the second edge 52 are dimensioned such that they may be attached to a vertex of the respective zig-zag structures 13, 15 of the stent-structure 1 to follow the stent-structure 1. The outer skirt 5 of this embodiment realizes the “Hawaiian skirt” version of attachment which is founded on the concept of intravalvular parietal skirt. The circumferential length (i.e. the width) of the outer skirt 5 shown in FIG. 28 equals the circumference of the stent-structure 1 at a belly level and at the inflow end of the flow passage or at the first stent-structure end 11. The height of the outer skirt 5 equals the height of the stent-structure between the inflow end 11 and the belly nadir.

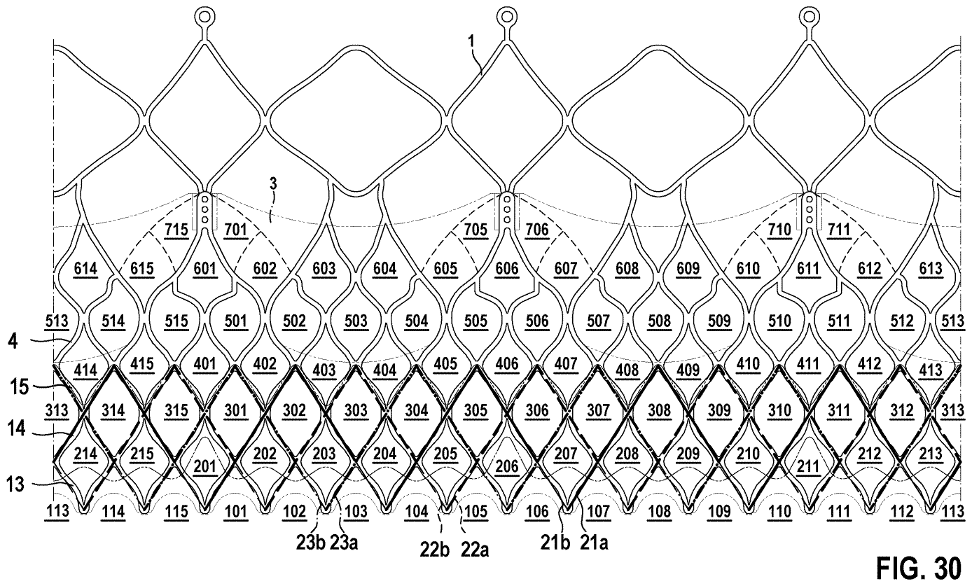

According to the first suture pattern shown in FIGS. 29 and 30 the outer skirt 5 (which may be a single piece of biological tissue or a polymer sheath) is sutured to the stent-structure along the three zig-zag structures 13, 14, 15. The suturing is provided by sutures, wherein each suture 21a, 22a, 23a extends over the three zig-zag structures 13, 14, 15. The lines 21a, 22a, 23a symbolize that the stitches for the sutures in these regions cross the outer skirt thickness and secure the outer skirt to the stent. The lines 21b, 22b, 23b symbolize that the sutures are only wrapped around the respective strut in this region of the stent structure without crossing the outer skirt 5. Accordingly, along the zig-zag structure 13 of the stent-structure 1 a fold creating section of the outer skirt 5 is formed.

By selectively limiting the suturing in the lowest zig-zag structure 13 of the stent-structure 1, the resulting unconstrained outer skirt 5 surface is folding when the prosthesis is crimped down to the compressed state. In this configuration, the outer skirt 5 assumes a shape that looks similar to a Hawaiian skirt. The Hawaiian skirt configuration is explained above in detail.

The outer skirt 5 may be made from pericardium such as porcine or bovine pericardium or from a fabric such as polyethylene or polyurethane. For sealing purposes, the second edge 52 of the outer skirt 5 may be sewn together with the inner skirt 32 or ribbon element.

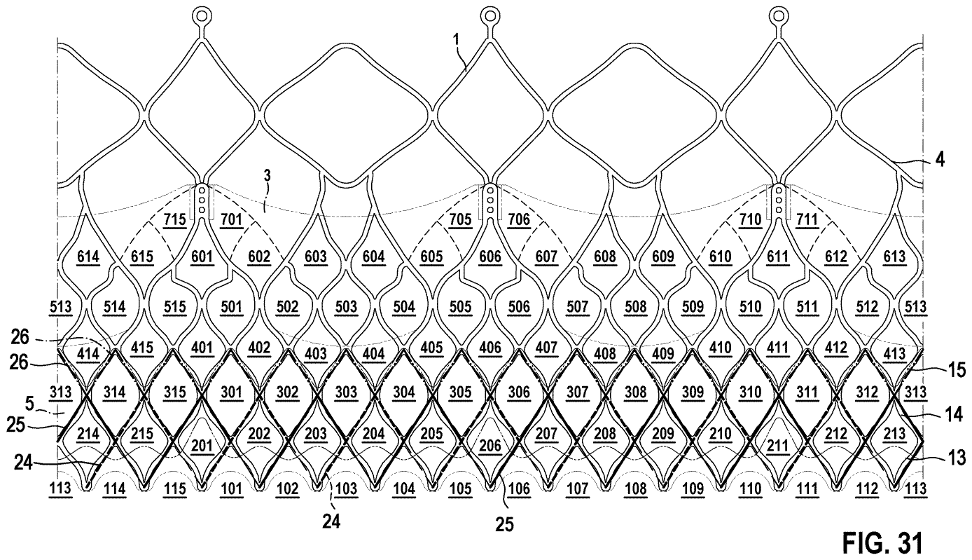

FIG. 31 shows a sewing pattern of the Hawaiian type in which a first suture 26 extends along the zig-zag structure 15, only (which is located between the cells 401 to 415 and the cells 301 to 315). The second and third sutures 24, 25 extend alternating along the zig-zag structures 13, 14 wherein, as explained above, along the zig-zag structures 14, 15 the outer skirt 5 is affixed to each strut forming the sealing section and along the zig-zag structure 13 the outer skirt 5 is affixed to each second strut, thereby forming the fold creating section.

The sutures 21, 22, 23 or 24, 25, respectively may be produced in a sequential manner around the full circumference such that a self-locking effect is achieved by the next sutures structure with regard to the already produced structure(s).

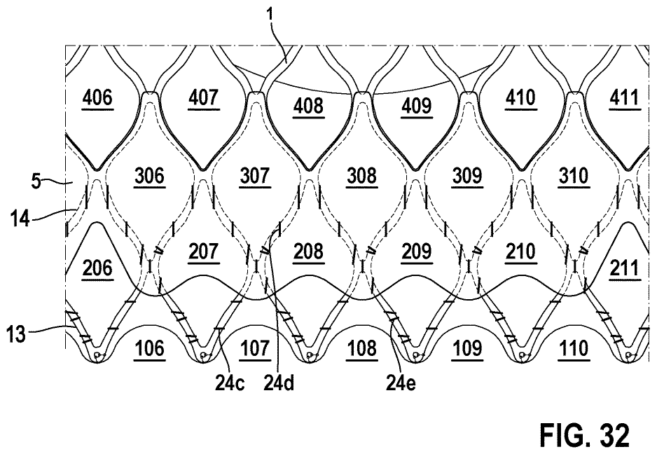

FIG. 32 shows that the direction of the thread forming the suture 24 may be different along the stent-structure 1. At the outer side of the stent-structure 1 the single stitches 24c of the suture 24 at the zig-zag structure 13 extend basically perpendicular to the longitudinal direction 18, whereas the single stitches 24d of the suture 24 at the zig-zag structure 14 extend basically along the longitudinal direction of the stent structure 1. Further, the arched stiches 24e along the struts on the right-hand side of cell 108 symbolizes that the respective suture 24 is wrapped around the respective strut without affixing the outer skirt 5 to the stent-structure 1 there.

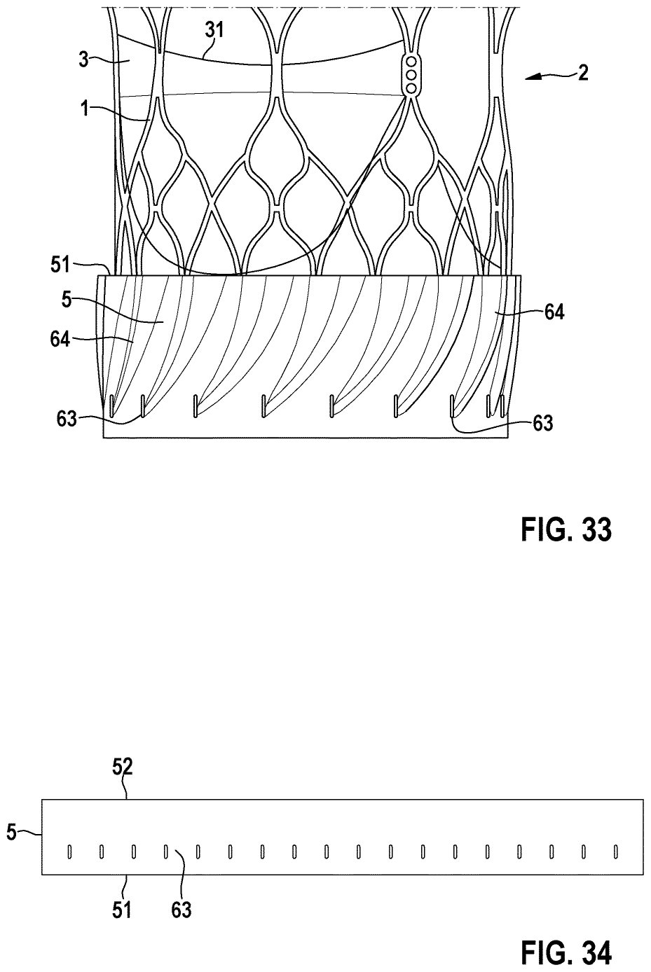

The second embodiment of a heart valve prosthesis is shown in FIG. 33 to 37. In contrast to the first embodiment, the fold creating section of the outer skirt is now located further to the outflow end of the flow passage than the sealing section.

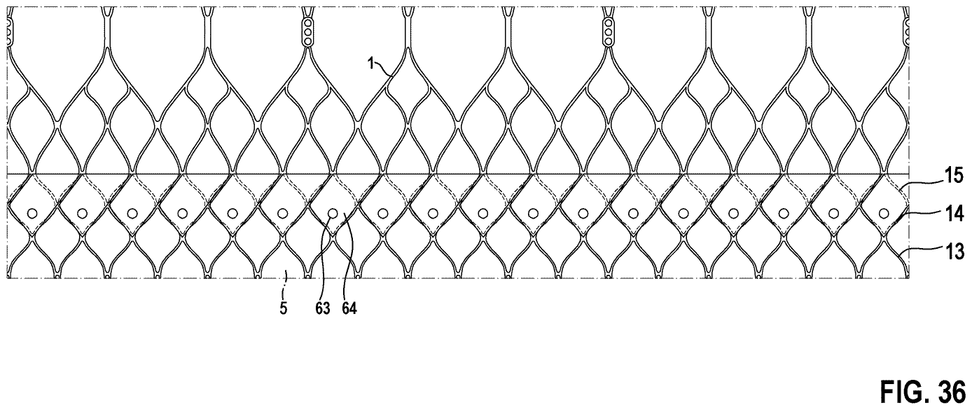

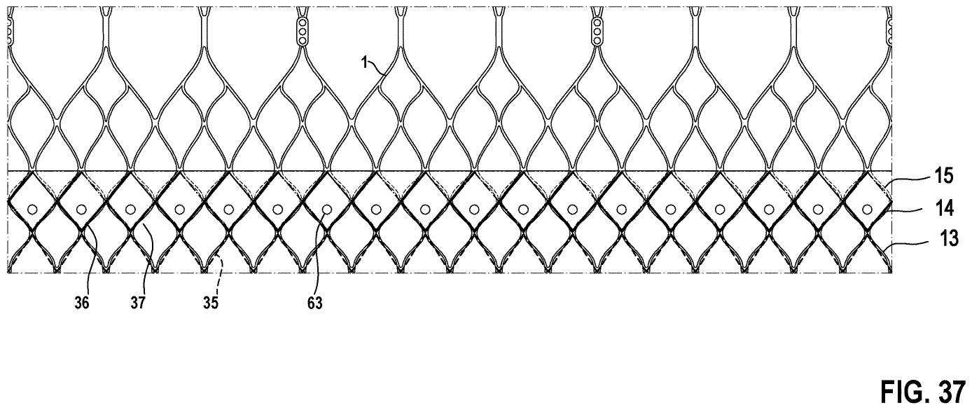

Each through hole 63 as shown in FIG. 33 is accommodated at an end of a conical, slightly sideways bent fold 64 forming a “bagpipe” structure. Accordingly, the trough holes are accommodated within the middle zig-zag structure 14 between the struts forming a “V” which is open into the direction of the outflow end of the flow passage. The through holes 63 may alternatively be replaced by slits or half-circles. In the Bagpipe skirt configuration blood is not intended to be trapped but redirected. The blood is channelled in the top openings of the outer skirt 5 through the fold 64 and redirected radially through the through holes 63, preferably side openings. The “bagpipe structure” is shown in FIG. 36 by the marked upper row. The outer skirt 5 of this embodiment is configured to predictably folds in bagpipe-like structures thanks to the specific sewing pattern shown in FIGS. 35A and 37.

The outer skirt 5 is shown in FIG. 34. The outer skirt tissue has a rectangular, straight form. The second edge 52 of the outer skirt 5 may be straight (as shown here) or scalloped. The first edge 51 at the inflow end of the flow passage can be selected from being saw-shaped, scalloped or straight. Further the outer skirt 5 has a row of slit-like through holes 63 located parallel to the second edge 52. The peaks of the scallops thereof have all the same height. For sealing purposes, the lower part of the outer skirt 5 close to the inflow end of the flow passage is sewn together with the inner skirt 32.

As shown in FIG. 35A the first embodiment of a sewing pattern may include three sutures 27, 28, 29 extending fully over the three adjacent zig-zag structures 13, 14, 15 at the first stent-structure end 11, extending circumferentially and crossing the full height of the outer skirt 5. The sutures 27, 28, 29 are provided sequentially around the circumference of the stent-structure 1 so that a self-locking effect of one suture by the next one is achieved. The two terminal, adjacent zig-zag structures 13, 14 are covered by the sealing section of the outer skirt 5 where the outer skirt 5 is sutured to each strut of these zig-zag structures 13, 14. There the continuous lines are used to indicate that the stitches go cross the outer skirt's 5 thickness and secure it on the stent-structure 1. At the upper zig-zag structure 15 which is located further to the outflow end of the flow passage, every second strut is not affixed to the stent-structure symbolized by the dashed line forming the fold creating section where the thread of the suture is wrapped around the struts without crossing the outer skirt 5. For better illustration of FIG. 35A the single sutures 27, 28, 29 are shown in FIGS. 35B, 35C and 35C, respectively.

The outer skirt 5 may be made from pericardium such as porcine or bovine pericardium or from a fabric such as polyethylene or polyurethane.

The sewing pattern shown in FIG. 37 includes sutures 35, 36, 37, wherein the suture 35 is located at the first stent-structure end 11 extending along the zig-zag structure 13, only. The other sutures 36, 37 extend over two zig-zag structures 14, 15 provided sequentially in order to achieve the above-mentioned self-locking effect. The location of the sealing section and the fold creating section are identical to the one of FIG. 35A.

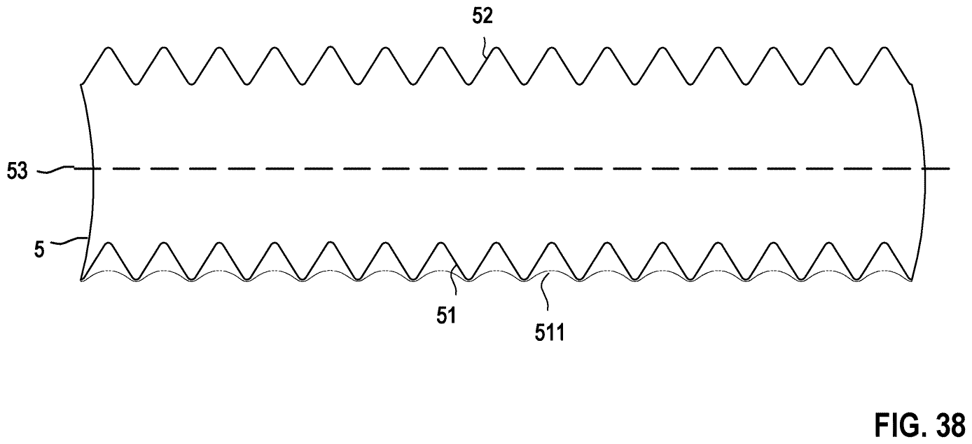

In a third embodiment of an inventive heart valve prosthesis having the so-called Primula configuration or cow-lip like shape is realized. An outer skirt 5 is shown in FIG. 38 which is (biological) tissue to be folded along the folding line 53 to form a hook like or loop like structure 7 (double layered outer skirt) or at the inflow end of the flow passage as depicted in FIG. 18B. The first edge 51 and the second edge 52 of the outer skirt 5 are saw-shaped. In another embodiment, the first edge 51 of the outer skirt 5 of the Primula configuration may be scalloped as depicted by the dashed line 51 in FIG. 38.

The outer skirt 5 is sutured sequentially using two sutures to the adjacent zig-zag structures 13, 14 located at the first stent-structure end 11 in order to achieve the self-locking effect. Thereby the sealing section is provided. Then, the outer skirt 5 is folded as shown in FIG. 18B so that a middle portion of the outer skirt 5 fully covers the sealing section from the outside. The section of the outer skirt 5 which is then located further to the outflow end of the flow passage is sutured by a third suture 40 to another zig-zag structure 15 which is located further to the outflow end of the flow passage than the zig-zag structures 13, 14. The third suture 40 sutures the outer skirt 5 to all struts of the zig-zag structure 15 along the full circumference.

The third embodiment aims to prevent and limit the occurrence of any PVL by augmenting the contact surface with the anatomical structures within the annular region and the neighbouring areas, subannular (LVOT) and suprannular (native leaflet).

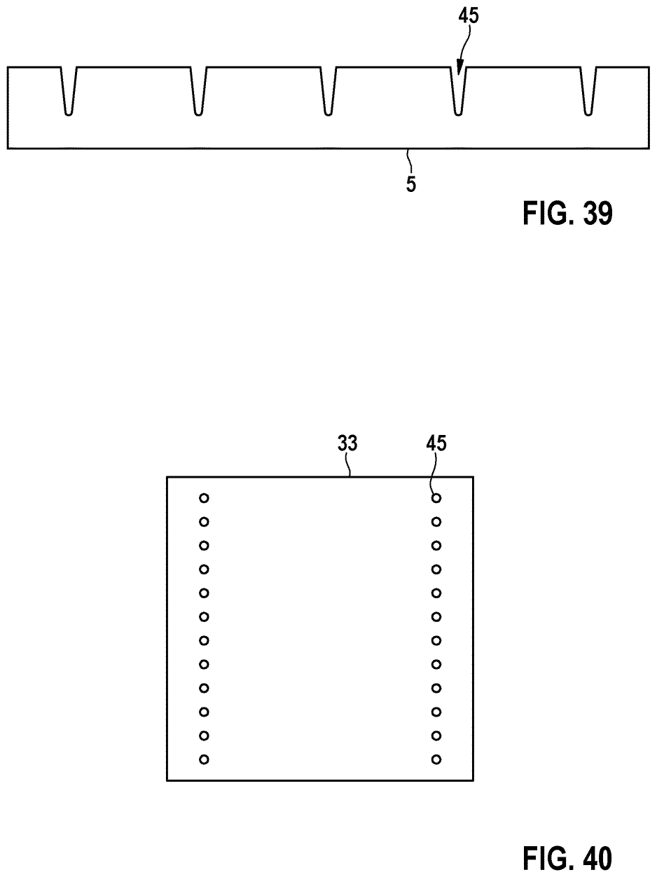

In order to further increase the quality of sutures and to ease the suturing process the outer skirt 5, 6 or the inner skirt 32 may include laser markings 45, each at a location where later a stitch may be provided. This is shown with regard to the outer skirt 5 in FIG. 39 as an example. Each laser marking 45 penetrates the thickness of the outer skirt 5 only partially. In a laser marking approach, the spot to be punctured is identified by locally engraving the surface of the biological tissue. Differently from a hole, a laser marking 45 preserves most of the biological structure of the tissue, minimizing therefore damages to the structural integrity of the material. As it can be observed, laser markings 45 only partially penetrate the tissue thickness. By appropriately tuning power and speed of the laser an adequate depth of the laser markings can be identified. Those marks shall be sufficiently big to be visible by the operators during the sewing process and at the same time sufficiently small to minimally impact the overall structure of the tissue. This can be achieved by appropriately tuning the speed and the power of the laser used to cut the tissue components, for example an inner skirt tissue 33. In order to create laser markings (L-ASP), little circles with diameter between 0.01 mm and 0.06 mm are used in the CAD software. Afterwards, the CAD file can be converted into the cutting file. The laser parameters may be chosen as follows: Power=3.5-7%, Speed=10%, PPI (point per inch)=500. However, it is necessary to consider the trade-off between the amount of circles and the error rate during importing of the data into the cutting file. The more circles on the cutting file are the higher the diameter has to be, in order to be fully imported into the laser cutter software as small circles lead to losses during importing into the cutting file. However, the circles should be small so that the laser markings 45 are not too big.

FIG. 40 shows an outer skirt having laser markings 45 which, when the outer skirt is folded can be sutured together.

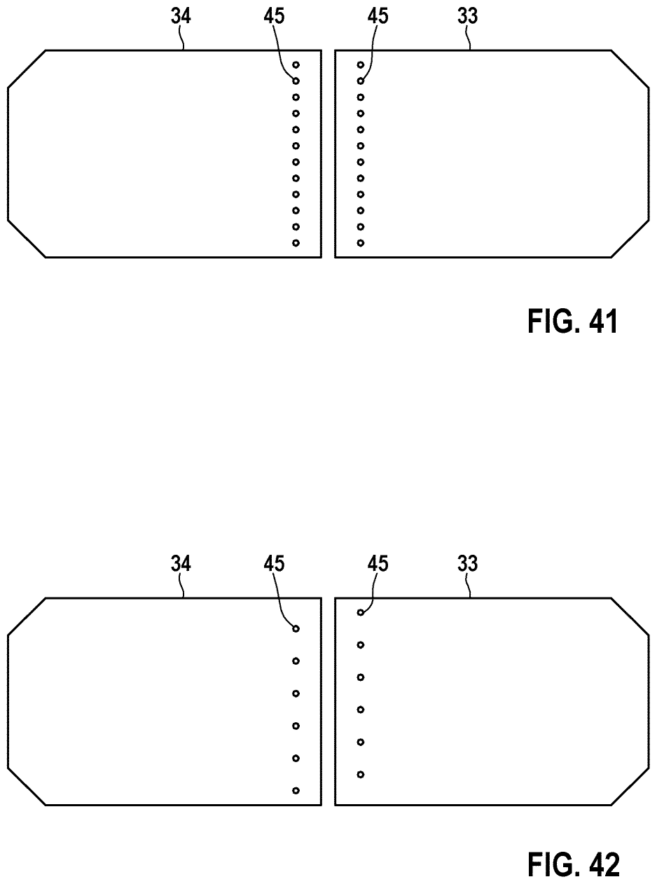

In FIGS. 41 and 42 two tissues 33, 34 are shown, respectively which needs to be sutured together. In FIG. 41 a double patterned topology and in FIG. 42 an alternating patterned topology of laser markings 45 is shown. The pitch of the laser markings 45 in FIG. 42 is double the pitch of the laser markings 45 of FIG. 41. With regard to FIG. 41 both tissues 33. 34 are aligned in a way that exactly two laser markings 45 lie on top of each other. This means that during the suturing process the needle goes through two laser markings 45 for each stitch. In the alternating patterned technology shown in FIG. 42 the laser markings 45 are separated with a distance of a double pitch length. With the correct alignment the tissues 33, 34 has a shifted positioning of the laser markings 45 relative to the reference layer so that the superposition of both tissues 33, 34 of both tissues 33, 34 gives the aimed pitch. This means that per stitch there is only one mark 45 that indicates the right spot.

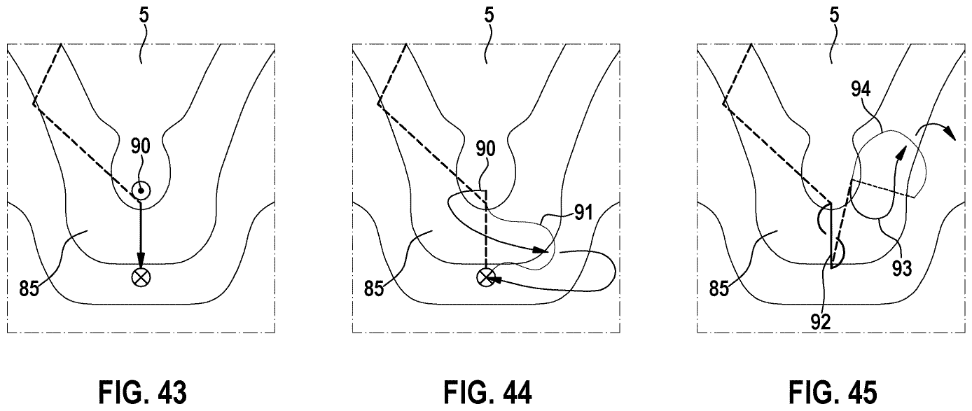

The suturing of the thread and an outer skirt (5, 6) to a terminal vertex such as a nutcracker eye 85 or a vertex 88 of two struts is illustrated in FIGS. 43 to 45 and FIGS. 46A to 46D, respectively. This suturing is capable to firmly connect the outer skirt 5 (e.g. a layer of soft material) to a strut of a rigid stent-structure.

The first embodiment refers to an inflow fixation on the nutcracker eye 85 includes executing a stitch from the inner side of the stent-structure through 90, whip the suture vertically around the eye 85, while going with the needle through the outer skirt 5 and let a loop 91 standing (see FIG. 43). Then come out at 90 to execute a single knot 92 and stitch again under the inflow strut (see FIG. 44). Stitch from inside closely under the bulk of the nut-cracker eye on the right side at position 93, whipping the suture horizontally around the inflow strut, while going with the needle through the outer skirt and let a loop 94 standing. Come out closely under the bulk again, use the created loop 95 to execute another single knot.

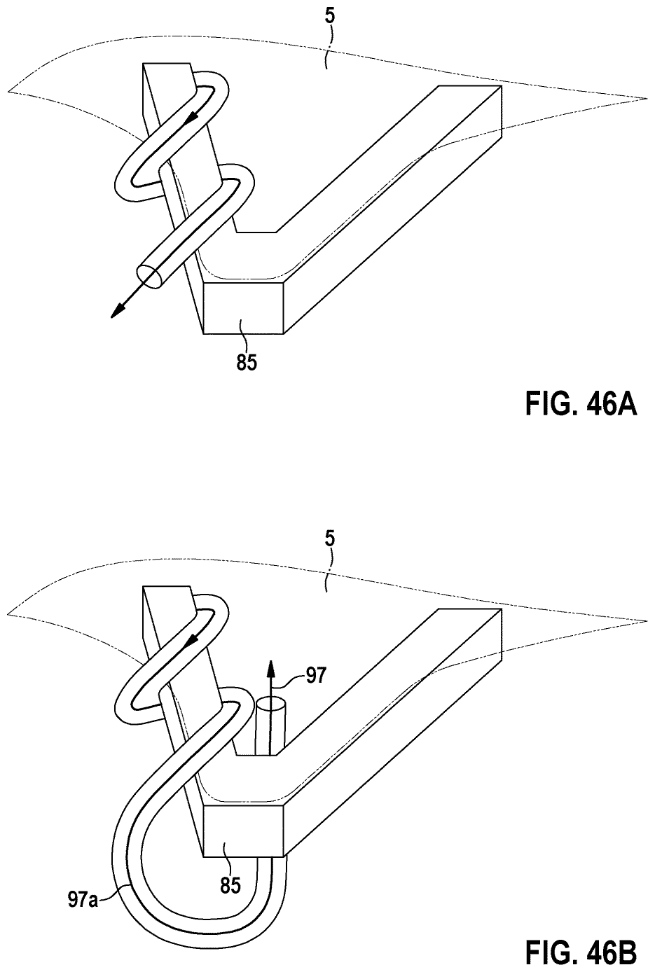

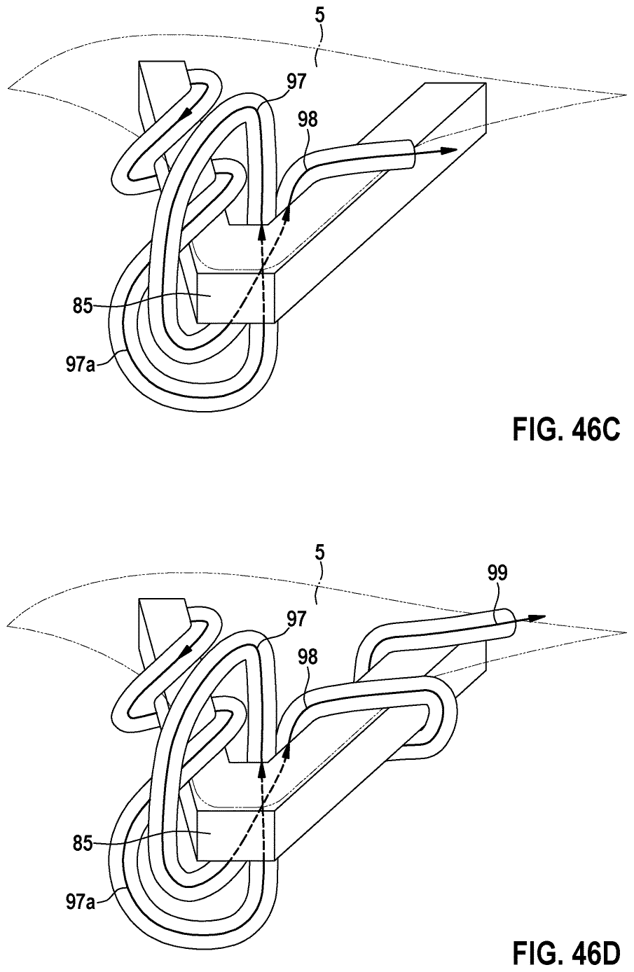

The second embodiment refers to the suturing of the inflow profile of an inner and/or outer skirt made, for example of pericardium, along the metallic stent-structure 1 zig-zag structure with struts at a vertex 88. The procedure has been divided into four steps of realization: The standard sewing of the outer skirt 5 by using whip stitches is performed until the vertex 88 of the zig-zag structure is reached: the suture shall be tightened (FIG. 46A). Then the suture is wrapped around the strut externally without puncturing the tissue of the outer skirt 5 and then the skirt is punctured with a different hole 97 (not the same hole of the previous whip stitch) from the inner side of the prosthesis, right at the center of the zig-zag structure vertex interspace. The suture shall not be tightened at this point, but instead a loose loop 97a shall be left open for the realization of the next step (FIG. 46B). Afterwards, the suture is again wrapped around the stent-structure externally, without puncturing the suture, and simultaneously crossed-over by passing through the loop 97a that has been left from the previous step (see above). The suture shall stay behind (towards the outflow, namely towards the center of the cell) the previous suture to allow the creation of the cross-over. The tissue is then punctured with a different hole 98 along the internal profile of the strut, on the other side of the cell (with respect to the whip stitches executed at the beginning of the procedure). Ideally, this suture hole 98 shall be placed symmetrically (mirrored) to the last whip stitch/hole in relation to the vertical (major) axis of the stent-structure rhombus/cell. At this point, there shall be 3 different suture holes (whip stitch hole, 97, 98) on the tissue surface of the outer skirt 5, within the zig-zag vertex interspace, placed at the interspace and forming an equilateral triangle (ideally). The suture shall be tightened at this point (see FIG. 46C). Then, the standard whip stitching 99 can continue along the stent-structure strut, according to the desired sewing pattern parameters and specifications (see FIG. 36D).

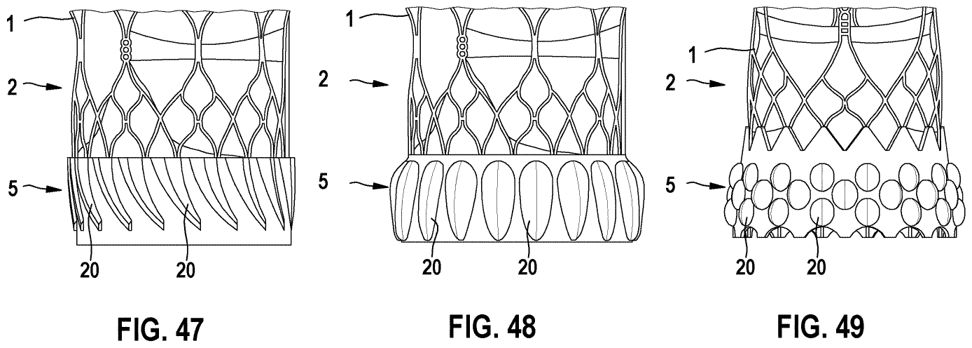

FIG. 47 to 49 show embodiments of medical implants 2 having a stent structure and 3D-shaped outer skirts 5 and optionally having a valve assembly 3. The outer skirts 5 have protrusions 20 on their outer surface. The outer surface of the outer skirt is the surface facing the anatomical structure (e.g. tissue, blood vessel or organ) at the implantations site. The outer skirts 5 may have cylindrical shape. The 3D-shapes were preformed during the manufacturing process of the outer skirt 5. The medical implants 2 may have compressed state and is expandable into a functional state. The medical implant 2 is shown in the (expanded) functional state. Furthermore, when the medical implant is crimped down to IFU implantation, the 3D structures might slightly readapting in shape due to the stent geometrical change.

FIG. 47 show a medical implant 2 having pleats, elongated channels or turbine-like structures on the outer skirt 5.

FIG. 48 show a medical implant 2 having peg-shaped protrusions on the outer skirt 5.

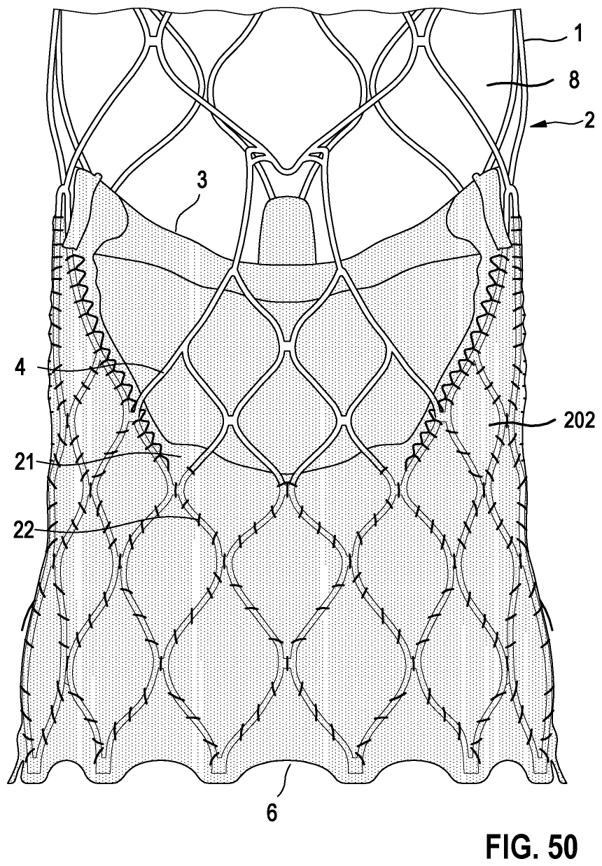

FIG. 49 shows a medical implant 2 having bubbles (spheres, spheroids, ellipsoids or shapes obtained by cutting such solids with a plane) on the outer skirt 5. The second end of the skirt may be saw-shaped to follow the stent. The second outer skirt 5 is sewn together with the inner skirt (not visible). The second end of the skirt at the outflow end of the flow passage can be saw-shaped (see FIG. 49), scalloped (see FIG. 48) or even flat (see FIG. 47).

FIG. 50 shows side view of another embodiment of a medical implant 2 in a functional state. The the medical implant 2, preferably a transcatheter deliverable endoprosthesis, includes a stent-structure 1, one skirt 6 and a valve assembly 3. The stent-structure has a first stent-structure end 11 and a second stent-structure end (not shown) being opposite to the first stent-structure end 11, and wherein the stent-structure 1 surrounds an inner volume 8 and wherein the stent-structure 1 has a plurality of struts 4 forming cells 101. The medical implant 2 includes only one skirt 6 partially covering an outer side of the stent-structure 1 which is fully wrapped around the first stent-structure end 11 and partially covers an inner side of the stent-structure 1. The valve assembly 3 is arranged within the inner volume 8 of the stent-structure. Depending on the how the skirt is affixed to the stent structure and/or whether the skirt has a 3D shaped structure, the skirt 6 partially covering the outer side may extend at least partly outside the stent-structure 1, preferably extends radially outward the circumference of the stent-structure 1. This means the the skirt 6 partially covering the outer side has protrusions. These protrusions may be pleats or suction cup shaped protrusions. These protrusions enable a better sealing towards the anatomical structure at the implantation site.

While specific embodiments of the present invention have been shown and described, it should be understood that other modifications, substitutions and alternatives are apparent to one of ordinary skill in the art. Such modifications, substitutions and alternatives can be made without departing from the spirit and scope of the invention, which should be determined from the appended claims.

Various features of the invention are set forth in the appended claims.

1. A medical implant for transcatheter delivery to an implantation site comprising:
an implant component defining a flow passage, the flow passage having an inflow end and an outflow end, wherein the implant component comprises a stent-structure,
wherein the stent-structure has a compressed state and is expandable into functional state, and wherein the stent-structure comprises a first stent-structure end, second stent-structure end opposite the first stent-structure end, a plurality of cells and a longitudinal axis,
an outer skirt at least partially covering an outer side of the stent-structure,
a suture affixing the outer skirt to the stent-structure,
wherein, at least in the functional state, the outer skirt comprises a plurality of protrusions forming a seamless 3D structure extending radially outward from a flat portion of the outer skirt, wherein the flat portion of the outer skirt abuts the stent-structure.

2. The medical implant according to claim 1, wherein 3D structure comprises a molded integral portion of the outer skirt that includes the plurality of protrusions.

3. The medical implant according to claim 1, wherein the protrusion comprises a bubble, sphere, spheroid, ellipsoid, or a shape generally obtained by cutting such solids with a plane, a fold or wrinkle, a polyhedron, a (elongated) channel, turbine-like structure, a wrinkle, a notch, a recess, a groove, or combinations thereof.

4. The medical implant according to claim 1, wherein the plurality of protrusions define pleats with recessions between portions of the pleats that extend radially outward.

5. The medical implant according to claim 4, wherein the pleats extend in the direction from the first stent-structure end to the second stent-structure end or the pleats extend in the direction of the longitudinal axis or the pleats are bent with an angle of less than 30° with respect to the longitudinal axis.

6. The medical implant according to claim 4, wherein the pleats comprise a horn-like or bagpipe shape.

7. The medical implant according to claim 6, wherein at least some or each of the horn-like or bagpipe shaped pleats form a top opening.

8. The medical implant according to claim 7, wherein at least some or each of the horn-like or bagpipe shaped pleats comprise a through hole, slit or side opening.

9. The medical implant according to claim 1, wherein the outer skirt comprises a first outer skirt edge and a second outer skirt edge and is folded between the first outer skirt edge and the second outer skirt edge to form a hook like or loop like structure.

10. The medical implant according to claim 9, comprising an inner skirt covering at least partially an inner side of the stent-structure.

11. The medical implant according to claim 10, wherein the first outer skirt edge is affixed to the inner skirt and/or the stent-structure and/or to the second outer skirt edge.

12. The medical implant according to any of claim 10, wherein the inner skirt covers the inner side of the stent-structure in the full circumference of the stent-structure and/or the outer skirt covers an outer side of the stent-structure in a full circumference of the stent-structure.

13. The medical implant according to claim 1, wherein the suture comprises an overlaying protecting stitch structure.

14. The medical implant according to claim 13, wherein the suture is arranged in one direction along the longitudinal axis and the protecting stitch structure is provided in an opposite direction relative to the one direction.

15. The medical implant according to claim 13, wherein the suture is formed at least partially by Armenian stitches and/or the protecting stitch structure is formed at least partially by whip stitches.

16. The medical implant according to claim 13, wherein the suture structure or the stitch structure extends through holes within the outer skirt at a vertex of the stent-structure and wherein the suture or the stitch structure forms a clove-hitch knot or wherein the suture or the stitch structure comprises two knots at a nutcracker eye at a first stent-structure end.

17. The medical implant according to claim 1, wherein the medical implant is a heart valve prosthesis, wherein the medical implant component comprises the stent-structure and the outer skirt and a valve assembly comprising at least two of leaflets, wherein the valve assembly is affixed to the stent-structure and extends at least partly within the stent-structure.

18. The medical implant according claim 17, wherein the outer skirt, the inner skirt and/or the one or more leaflets comprise one or more laser markings or laser holes.

19. The medical implant according to claim 1, wherein the outer skirt comprises a saw-shaped or scalloped or straight first edge and/or a saw-shaped or scalloped or straight second edge.

20. A manufacturing method for an implant for transcatheter delivery to an implantation site comprising:
providing an implant component defining a flow passage, the flow passage having an inflow end and an outflow end, wherein the medical implant component comprises a stent-structure,
wherein the stent-structure has a compressed state and is expandable into functional state, and
wherein the stent-structure comprises a first stent-structure end, second stent-structure end opposite the first stent-structure end, a plurality of full cells and a longitudinal axis, wherein each full cell comprises two zig-zag structures formed by a plurality of struts, wherein the vertices of the zig-zag structures are coupled together, wherein each zig-zag structure extends around the full circumference of the stent-structure with regard to the longitudinal axis,
3D shaping an outer skirt to comprise a plurality of protrusions forming a seamless 3D structure extending radially outward from a flat portion of the outer skirt,
affixing the outer skirt to the stent-structure using a suture in at least a sealing section and a fold creating section such that it extends at least partly outside the stent-structure and such that in the sealing section the outer skirt is sutured to each strut of at least two directly adjacent zig-zag structures along the full circumference of the stent-structure and
such that in the fold creating section the outer skirt is sutured to at least part of the struts of one zig-zag structure around the full circumference of the stent-structure and such that the outer skirt forms a fold with the diameter enlarging 3D structure by interaction with the fluid flow when the medical implant is placed at the implantation site and the flat portion of the outer skirt abuts the stent-structure.
